(19) United States
(12) Patent Application Publication
(10) Pub. No.: US 2023/0364243 A1
(43) Pub. Date: Nov. 16, 2023

(54) CANCER-SELECTIVE TARGET DEGRADATION BY TARGETING GROUP CAGED PROTACS

(71) Applicants: Beth Israel Deaconess Medical Center, Inc., Boston, MA (US); Icahn School of Medicine at Mount Sinai, New York, NY (US)

(72) Inventors: Wenyi WEI, Boston, MA (US); Jian JIN, New York, NY (US); Husnu Ümit KANISKAN, New York, NY (US); He CHEN, New York, NY (US); Jing LIU, Boston, MA (US)

(73) Assignees: Beth Israel Deaconess Medical Center, Inc., Boston, MA (US); Icahn School of Medicine at Mount Sinai, New York, NY (US)

(21) Appl. No.: 18/362,634

(22) Filed: Jul. 31, 2023

Related U.S. Application Data
(63) Continuation of application No. PCT/US2022/014325, filed on Jan. 28, 2022.
(60) Provisional application No. 63/144,442, filed on Feb. 1, 2021.
(60) Provisional application No. 63/186,005, filed on May 7, 2021.

(51) Int. Cl. A61K 47/55; C07D 475/04

(52) U.S. Cl. CPC A61K 47/55; C07D 475/04

(57) ABSTRACT

PROTACs (PROteolysis TArgeting Chimeras) are an emerging class of promising therapeutic modalities that degrade intracellular protein targets by hijacking the cellular ubiquitin-proteasome system. However, potential toxicity of PROTACs in normal cells due to off-tissue on-target degradation effect limits their clinical applications. Precise control of PROTAC's on-target degradation activity in a tissue selective manner could minimize potential toxicity/side-effects. To this end, we developed a cancer cell selective delivery strategy for PROTACs by conjugating a folate group to ubqiquitin recruitment moiety to achieve targeted degradation of proteins of interest (POIs) in cancer cells versus normal cells. We show that our folate-PROTACs, including BRD PROTAC (Folate-ARV-771), MEK PROTAC (Folate-MS432) and ALK PROTAC (Folate-MS99, Folate-S2-MS4048) are capable of degrading BRDs, MEKs and ALK, respectively, in a folate receptor-dependent manner. This design provides a generalizable platform for PROTACs to achieve selective degradation of proteins of interest (POIs) in cancer cells.

### CROSS-REFERENCE TO RELATED APPLICATIONS

This application is a continuation under 35 U.S.C. § 111(a) of PCT International Patent Application No. PCT/US2022/014325, filed Jan. 28, 2022, designating the United States and published in English, which claims priority to and the benefit of U.S. Provisional Application No. 63/144,442, filed Feb. 1, 2021, and U.S. Provisional Application No. 63/186,005, filed May 7, 2021, the entire contents of each of which are incorporated by reference herein.

### STATEMENT OF RIGHTS TO INVENTIONS MADE UNDER FEDERALLY SPONSORED RESEARCH

This invention was made with government support under Grant No. CA253027 awarded by the National Institutes of Health. The government has certain rights in this invention.

### SEQUENCE LISTING

The present application contains a Sequence Listing which has been submitted electronically in XML format following conversion from the originally filed TXT format.

The content of the electronic XML Sequence Listing, (Date of creation: Jul. 29, 2023; Size: 4,815 bytes; Name: 167688-020403US-Sequence Listing.xml), and the original TXT format, is herein incorporated by reference in its entirety.

### BACKGROUND

PROteolysis TArgeting Chimera (PROTAC) technique emerged as the result of identifying peptides or small molecule chemical ligands that specifically bind with endogenous E3 ligases, such as β-TRCP, the von Hippel-Lindau tumor suppressor (VHL), Mdm2 and Cereblon (CRBN). Structurally, PROTAC is a bifunctional small molecule that consists of two functional parts, a “warhead” that displays high specificity in binding a protein of interest (POI), and a ligand that is recognized by E3 ligase, where the warhead and the E3 ligase ligand are connected by a linker. PROTACs dictate the POI for proteolysis allowing for the compound to target non-catalytic enzymes with concomitant reduction in drug exposure time and dosage required to suppress signaling. Additionally, PROTACs may eliminate both the catalytic activity and the scaffolding function of bifunctional proteins, such as Receptor Tyrosine Kinases (RTKs).

By hijacking an endogenous E3 ubiquitin ligase and the ubiquitin-proteasome system (UPS), the PROteolysis TArgeting Chimera technology could potentially be applied to target any intracellular proteins for degradation, including those so-called undruggable targets such as transcriptional factors and scaffold proteins. Compared to small-molecule inhibitors, PROTACs are potentially more powerful therapeutic modalities, as they do not rely on occupancy-driven pharmacology, in part due to the catalytic nature of PROTACs in degrading their protein targets. However, potential off-tissue effect(s), i.e. diffused distribution of PROTAC molecules in non-target normal tissue/organs after systemically administered, limit its application in clinic.

However, when systemically administered, PROTACs could result in the uncontrolled degradation of POIs in any cells it can access. For example, inhibition of BET bromodomains is relatively well-tolerated while complete loss of BRD2 and BRD4 is lethal. Accordingly, methods for regulating the spatial and/or temporal activation of PROTACs in select tissues and cells are urgently required.

Light-controllable PROTACs have been developed, either by using caging groups, such as nitroveratryloxycarbonyl (NOVC) and [7-(diethylamino)coumarin-4-yl]methyl (DEACM) or by using photo-switchable compound, such as azobenzene installed on the ubiquitin recruitment moiety of the PROTAC, which renders the compounds inert until cleavage of the caging group. These controllable PROTACs provide an avenue to achieve spatiotemporal regulation of the catalytic activity of the PROTAC molecule. However, due to limits of light transmission, these can be only used in light accessible cancer types with clear boundary between tumor and adjacent normal tissue, such as skin cancer. Thus, developing an alternative method to specifically deliver PROTACs into cancer cells, whicle maintain PROTAC functionality to achieve controllable targeted degradation of protein of interest (POI) by PROTACs is needed. Such alternative methods may eliminate potential toxicity to normal tissues or cells.

### SUMMARY

In accordance with the foregoing objectives and others, the present disclosure provides compounds capable of recruiting ubiquitin in cells following endocytosis of the compound. The compound may interact with the hydrolases in the cells in order to cleave a moiety from the ubiquitin recruitment portion of the PROTAC thereby activating the ability of the compound to degrade the protein of interest cell following entry. The degradation of these compounds allows the compounds to recruit ubiquitin and cause degradation of a protein of interest and resultant cell death. For example, by installing a folate conjugated ester on the (R)-hydroxyl group of VHL ligands that recruit VHL E3 ligase, the endocytosis of the PROTAC may occur thereby allowing for activation of the ubiquitin reqruitment following entry to the cell. In the absence of hydrolysis, the compounds are inert (or with significantly decreased E3 ligase recruitment activity). Compounds of the disclosure comprise targeting groups, conjugated to a PROTOAC which block the proteolytic activity until cleavage of the targeting group (e.g., cleavage through interaction with endogenous hydrolases following endocytosis) permits proteolysis of a protein of interest (POI).

A typical PROTAC molecule consists three functional parts, a warhead to recruit the POI, a ligand to recruit the E3 ligase, and a linker between these two moieties. The compounds of the present disclosure include a targeting ligand appended to the ubiquitin recruitment moiety. The targeting group may be selected to aid in endocytosis of the compound. For example, the targeting group may be chosen to specifically bind to one or more proteins expressed on the cellular membrane and aid in the endocytosis of the compounds. These cellular membrane proteins may have increased expression in cancer cells as compared to healthy cells. For example, a folate group may be installed onto the ligand of E3 ligase or a glutaride nitrogen of a molecular glue. For VHL-based PROTAC, the hydroxyl group in VHL ligand is critical for the recruitment of Von Hippel-Lindau (VHL) E3 ligase, and inversion of the stereochemistry or installation of a bulky caging group on the hydroxyl moiety abolishes PROTAC activity. Without wishing to be bound by theory, the compounds of the present disclosure may have the following characteristics: (1) the targeting group (e.g., folate moiety) aids targeted enrichment of PROTACs into cancer cells; and (2) similar to the other caging strategies, the caged folate-PROTAC compound is inert to begin with and can be activated after being uncaged via cleavage by endogenous hydrolases or reductases (e.g., GSH) in cells. An exemplary schematic of this may be seen for Compound 1 (Folate-ARV-771) in FIGS. 1A and 1B).

The targeting group conjugated PROTAC of the present disclosure include compounds, wherein ubiquitin recruitment for the PROTAC only occurs following hydrolytic cleavage of the targeting group. The targeting group may preferentially bind to certain types of cells (e.g., the cellular membrane of cells), such as cancer cells, which express specific proteins on their cellular membrane as compared to normal or health cells. Such cellular membrane binding may facilitate endocytosis of the PROTAC compounds. In some embodiments, the targeting group of the PROTAC comprises a folate moiety. In some embodiments, the PROTAC is conjugated to a fluorodeoxyglucose moiety, biotin moiety, or other targeting group that preferentially binds to the cellular membrane of cancer cells to facilitate endocytosis.

These compounds may have the structure of formula (I):

PB-L₁-ULB-L₂-TG (I)

wherein ULB is a ubiquitin ligase binding moiety;
L₁ is absent or a linker;
L₂ is absent or a linker;
PB is a protein binding moiety; and
TG is a targeting group that preferentially binds to a protein with increased expression in a neoplastic cell as compared to an otherwise identical healthy cell;
   wherein ubiquitin ligase binding potential of said compound is increased following cleavage between the ULB group and the TG group;
   or pharmaceutically acceptable salts thereof. In some embodiments, the cleavage between TG and ULB may be induced byan hydrolase or reductase (e.g., an endogenous hydrolase or reductase) in the neoplastic cell. In some embodiments, TG is a folate moiety or a fluorodeoxyglucose moiety or a biotin moiety. For example, the TG group may be a folate or folic acid derivative having the structure of formula (II):

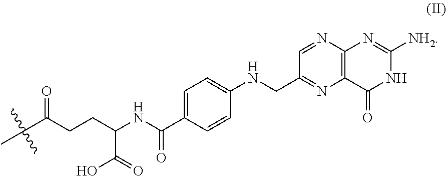 [SMILES: *C(=O)CCC(NC(=O)c1ccc(NCc2cnc3nc(N)[nH]c(=O)c3n2)cc1)C(=O)O]

wherein

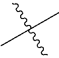

the point of attachment to the compound. In some embodiments, the folate derivative or folic acid derivative may have the structure of formula (IIa) or (IIb):

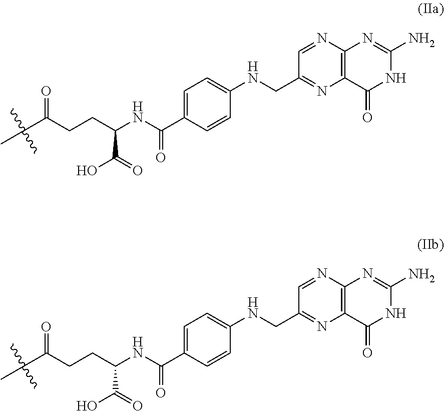 [SMILES: *C(=O)CC[C@@H](NC(=O)c1ccc(NCc2cnc3nc(N)[nH]c(=O)c3n2)cc1)C(=O)O ; *C(=O)CC[C@H](NC(=O)c1ccc(NCc2cnc3nc(N)[nH]c(=O)c3n2)cc1)C(=O)O]

wherein

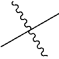

the point of attachment to the compound.

The -L₂-TG portion of the compounds of the present disclousure moieties may be conjugated to the ULB through a hydroxyl group typically present on an unconjugated ULB moiety, where that hydroxyl group is required for ubiquitin ligase binding such as an (R)-hydroxy group. In some embodiments, the the conjugation through the hydroxyl group is ester conjugation (e.g., —OH of the unconjugated ULB parent compound is replaced with —OC(O)-L₂-TG).

Typically, ULB binds to an E3 ubiquitin ligase following cleavage. In some embodiments, ULB does not bind (or has decreased binding) to an E3 ubiquitin ligase prior to cleavage. The E3 ubiquitin ligase may be selected from the group consisting of von Hippel Lindau (VHL) E3 ubiquitin ligase, β-Transducin Repeat Containing (β-TRCP) E3 Ubiquitin Protein Ligase, Mouse Double Minute 2 (Mdm2) E3 Ubiquitin Protein Ligase, and a Cereblon (CRBN) E3 Ubiquitin ligase.

The compound may have the structure of formula (III):

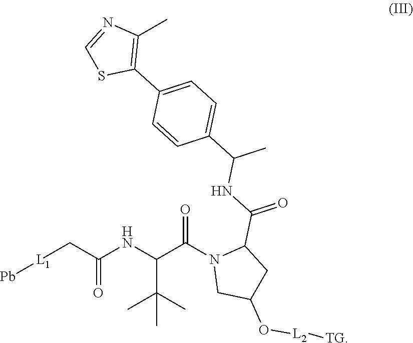

In some embodiments, the compound has the structure of formula (IIIa), (IIIb), (IIIc), (IIId), or (IIIe):

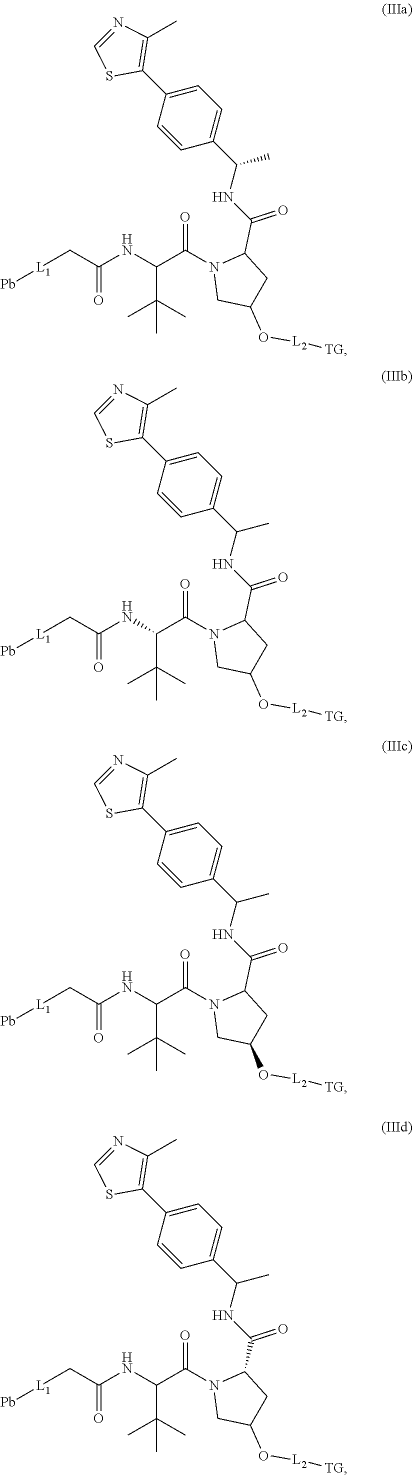

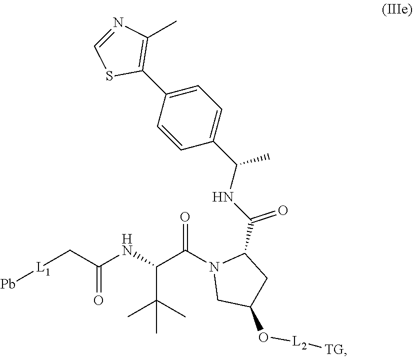

In some embodiments, the ULB is an immunomodulatory imide drug (IMiD) such as those and derivatives of those described in Ito, T. et al Science 327 (2010): 1345-1350, Kronke, J et al Science 343 (2014): 301-305, and Lu, G. et al Science 343 (2014): 305-309, each of which are hereby incorporated by reference in their entirety and particularly in relation to IMiDs, sulfonamide such as those and derivatives of those described in Han, T. et al Science 356 (2017), and Uehara, T. et al Nat Chem Biol 13 (2017): 675-680, each of which are hereby incorporated by reference in their entirety and particularly in relation to sulfonamide based degradaders, or cyclin K degrader such as those and derivatives of those described in Slabicki, M. et al Nature 585 (2020): 293-297, Mayor-Ruiz, C. et al. Nat Chem Biol 16 (2020): 1199-1207, and Lv, L. et al Elife 9 (2020): e59994, each of which are hereby incorporated by reference in their entirety and particularly in relation to cyclin K degraders. In some embodiments, ULB is lenalidomide derived (e.g., conjugated at two hydrogen positions of optionally substituted 3-(4-amino-1-oxoisoindolin-2-yl)piperidine-2,6-dione), pomalidomide derived (e.g., conjugated at two hydrogen positions of optionally substituted 4-amino-2-(2,6-dioxopiperidin-3-yl)isoindoline-1,3-dione), or thalidomide derived (e.g., conjugated at two hydrogen positions of optionally substituted 2-(2,6-dioxopiperidin-3-yl)isoindoline-1,3-dione).

In some embodiments, ULB comprises a piperidine dione and L₂ is conjugated to ULB through the piperdine dione. For example, the compound may have the structure of formula (IV):

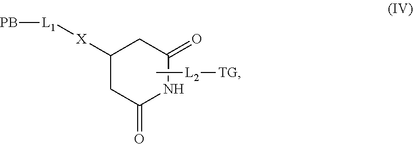

wherein X is absent (i.e, it is a bond) or may comprise the remaining portions of the ULB moiety (e.g., optionally substituted isoindolin-1-one such as 4-aminoisoindolin-1-one, optionally substituted isoindolin,1-3-dione such as 4-aminoisoindolin1,3,dione) which is conjugated to the L₁ moiety. In some embodiments, the compound has the structure of formula (IVa):

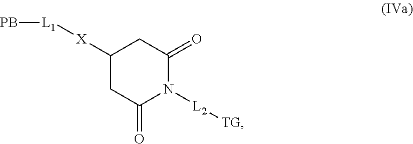

In certain implementations, the compound has the structure of formula (IVb):

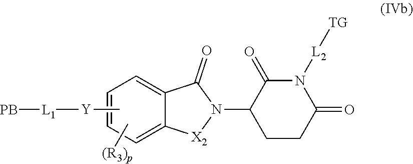

wherein p is 0, 1, 2, or, 3;
R³ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.);
X₂ is C(O), CH, CR^{a}, or NR^{a};
Y is absent (i.e, a bond), —O—, —C(O)—, —NR^{a}—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₃ alkyl, etc.). Compounds having the structure of formula (III) have the PLG group linked to the ULB through the glutarimide nitrogen. For example, the ULB is lenalidomide derived, pomalidomide derived, or thalidomide derived. In various aspects, the ULB is lenalidomide derived and the compound has the structure of formula (IVc) or (IVd):

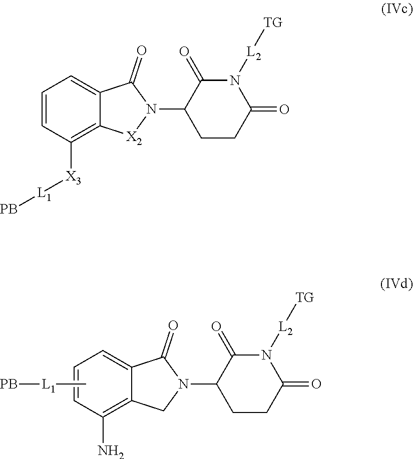

wherein X₃ is —NH— or —O—.

In some embodiments, the ULB moiety is thalidomide derived and the compound has the structure of formula (IVe):

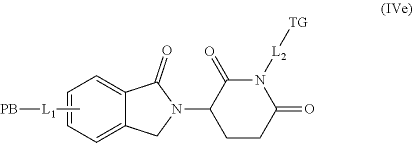

In specific embodiments, ULB moiety is pomalidomide derived and the compound has the structure of formula (IVf) or (IVg):

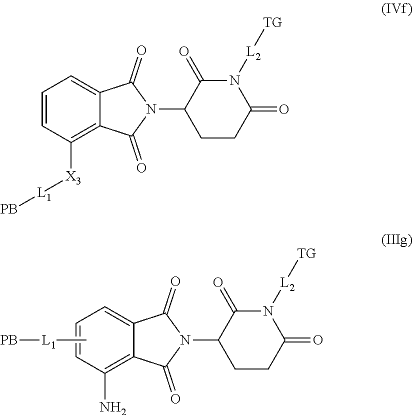

wherein X₃ is —NH— or —O—.

The L₂ linking moiety links the targeting group to the ULB group. The L₂ linking moiety may be chosen such that the compound has one or more of the following characterstics:
1) after the addition of the targeting group, the PROTAC is inactive; and
2) upon decaging (or hydroxylase or reductase-mediated cleavage), the resulting PROTAC can be fully active or require additional cleavage to become fully active.

In some embodiments, L₂ comprises a heteroarylene group, —S—S—, —C(O)—, —NH—, or combinations thereof. In some embodiments, L₂ comprises a heteroarylene group (e.g., a five membereed heteroarylene, a six membered heteroarylene, a divalent triazole, a divalent imidazole, a divalent pyrrole).

In various implementations, the compound has the structure of formula (V) (i.e., L₂ may be a group —X₁—X₂—X₃—X₄—X₅—X₆—X₇—):

PB-L₁-ULB—X₁—X₂—X₃—X₄—X₅—X₆—X₇-TG (V)

wherein X₁-X₇ are independently selected from absent (i.e., it is a bond), —C(O)—, —O—, —OC(O)—, —NR^{a}—, —N(R^{a})C(O)—, —(C(R^{a})(R^{a}))₁₋₈—, —(C(R^{a})(R^{a})C(R^{a})(R^{a})O)₁₋₈, —S—S—, —S—, arylene (e.g., C₅-C₆ arylene), and heteroarylene (e.g., five or six membered heteroarylene); and
R^{a} is independently selected at each occurrence from hydrogen and alkyl (e.g., C₁-C₄ alkyl).

In some embodiments, X₁ is —C(O)— or —C(O)O—. In some embodiments, one of X₃, X₄, or X₅ is heteroarylene (e.g., a five membered heteroarylene, a six membered heteroarylene) such as those produced in an alkyne-azide reaction. For example, X₃, X₄, or X₅ may be

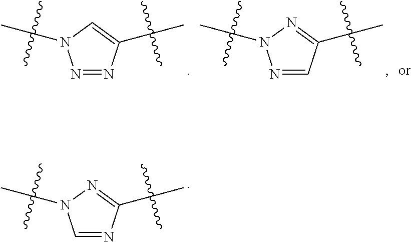 [SMILES: *c1cn(*)nn1 ; *c1cnn(*)n1 ; *c1ncn(*)n1]

In certain implementations, the linker moiety conjugated to TG (e.g., X₅, X₆, X₇) is —NR^{a}—.

In some embodiments, the compound has the structure of formula (Va):

PB-L₁-ULB—X₁—X₂—X₃—X₄—X₅-TG. (Va)

The compound may have the structure of formula (Vb):

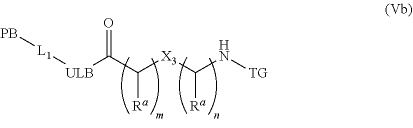

wherein m and n are independently an intenger selected from 0-8 (i.e., 0, 1, 2, 3, 4, 5, 6, 7, and 8) and
X₃ is heteroarylene (e.g., a five membereed heteroarylene, a six membered heteroarylene, a divalent triazole, a divalent imidazole, a divalent pyrrole). In some embodients, the compound has the structure of formula (Vc):

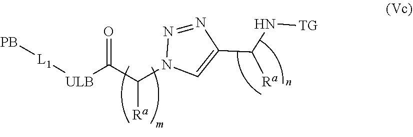

The L₂ linker may be chosen such that a portion of the L₂ moiety can be reduced by intracellular gluthianone (GSH) following endocytosis of the compound into a cell. Intracellular creation of an active PROTAC may via the formation of one or more steps within the cell. For example, the active (or decaged) PROTAC may be produced with the following synthetic mechanism (e.g., when using disulfide based linkers):

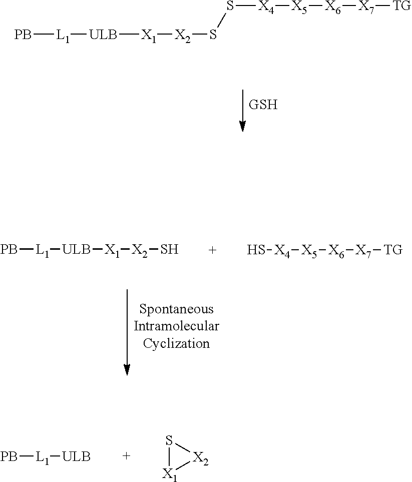

As depicted the scheme above, the targetting group caged PROTAC, may comprise a disulfide linker such that reduction by GSH within the cell separates the PROTAC from the targeting group cage to form a PROTAC conjugated to a thiol moiety. Subsequently, spontaneous intramolecular cyclization may produce an active PROTAC (PB-L₁-ULB) and a cyclized byproduct. In the scheme outline above, X₃ is chosen as the disulfide linker. In some embodiments, any of X₃-X₇ may be the disulfide linker such that the spontaneous intramolecular cyclization. In particular embodiments, L₂ is —C(O)O—(C(R^{a})₂)₂₋₃—S—S—X₄—X₅—X₆—X₇ such that the cyclized byproduct is optionally substituted 1,3-oxathiolan-2-one or optionally substituted 1,3-oxathian-2-one. An exemplary intracellular decaging could be:

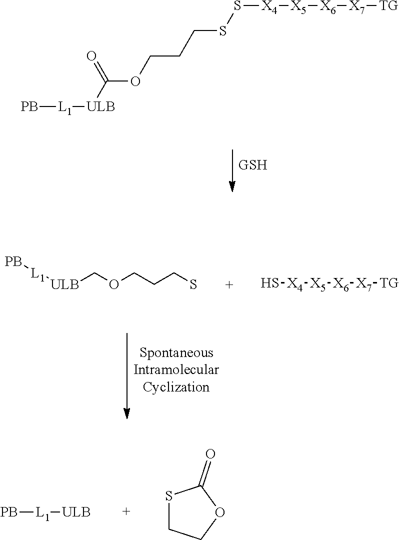

In some embodiments, and particularly embodiments having the structure of formula (IV), L₂ comprises a disulfide linker (e.g., one of X₁-X₅ in formula (V) is —S—S—). For example, the compound may have the structure of formula (Vd):

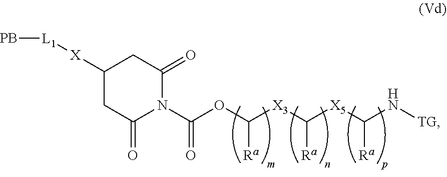

wherein m, n, and p are independently an intenger selected from 0-8 (i.e., 0, 1, 2, 3, 4, 5, 6, 7, and 8); and
X₃ or X₅ is —S—S—. In some embodiments, one of X₃ or X₅ is —S—S— and the other of X₃ or X₅ is X₃ is heteroarylene (e.g., a five membereed heteroarylene, a six membered heteroarylene, a divalent triazole, a divalent imidazole, a divalent pyrrole). In certain aspects, the compound may have the structure according to formula (Ve):

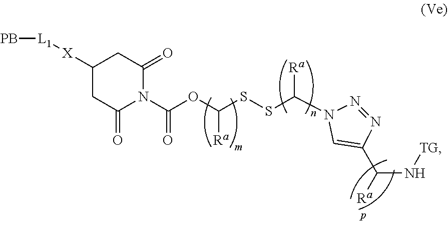

In some embodiments, the compound has the structure of formula (VI), (VIa), (VIb), (VIc), (VId) or (VIe):

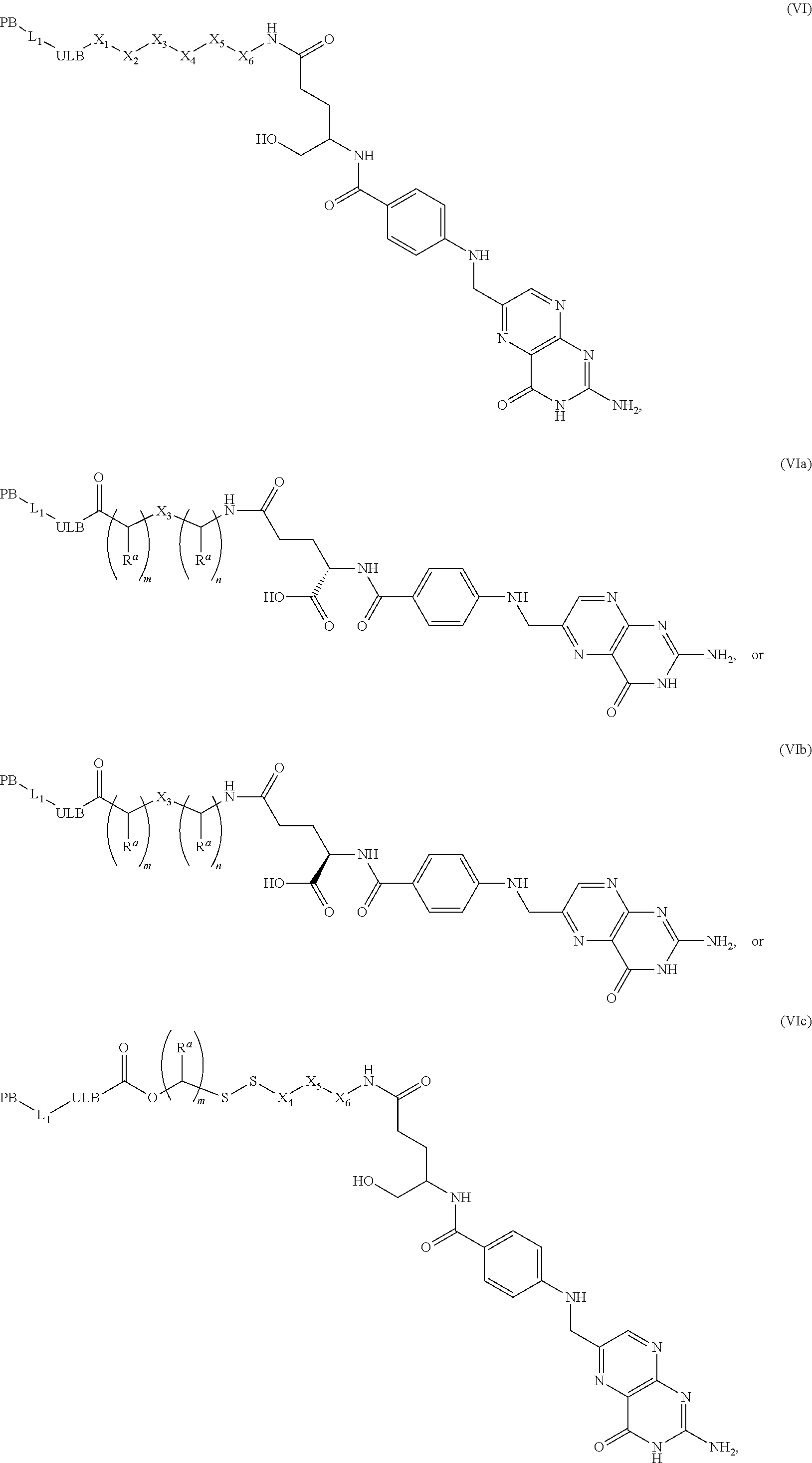

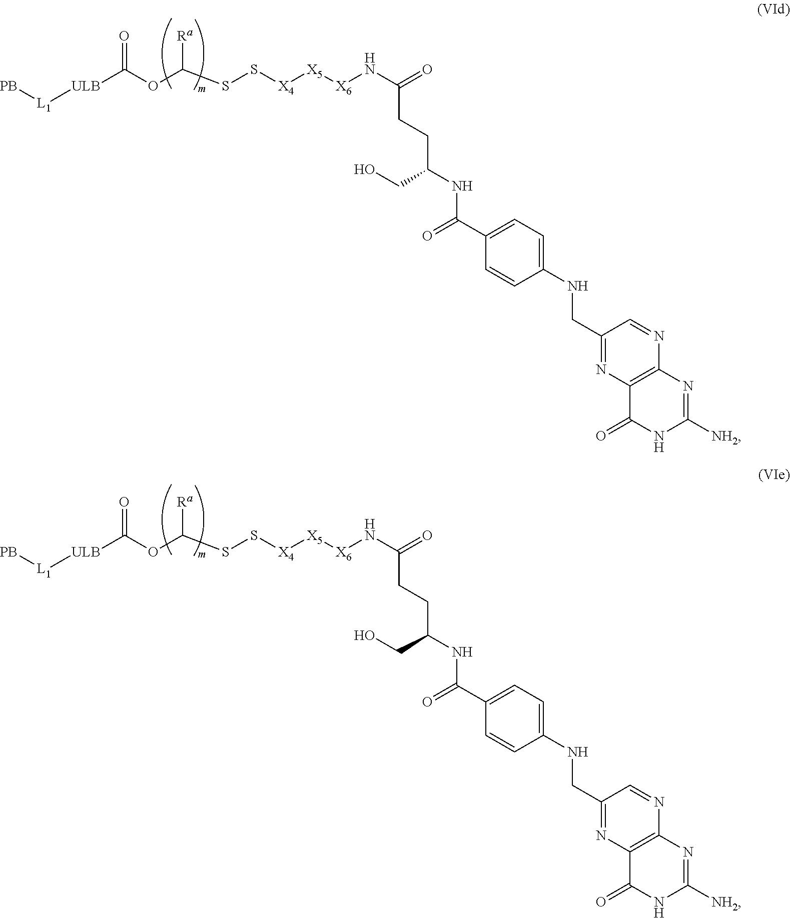

wherein m and n are indpendently an integer selected from 0-8;
X₃, X₄, or X₅ is heteroarylene (e.g., a five membereed heteroarylene, a six membered heteroarylene, a divalent triazole, a divalent imidazole, a divalent pyrrole); and
R^{a} is independently selected at each occurrence from hydrogen and alkyl (e.g., C₁-C₄ alkyl).

One functional part of the targeting group conjugated PROTAC is the protein binding moiety which binds specifically to a POI. The protein binding moiety may be a tyrosine kinase inhibitor, a BRAF-mutant inhibitor, or a MEK inhibitor. In some embodiments, the protein binding moiety binds to one or more of Abelson Murine Leukemia (ABL) Proteins, Breakpoint Cluster Region Protein (BCR), BCR-ABL fusion proteins, Bromodomain and Extra Terminal Domain (BRD) Family proteins, anaplastic lymphoma kinase (ALK) protein, echinoderm microtubule-associated protein like (EML)-ALK fusion proteins. In various implementations, PB has an affinity for its target protein (K_{d}) of less than 1 mM. In some embodiments, PB is:

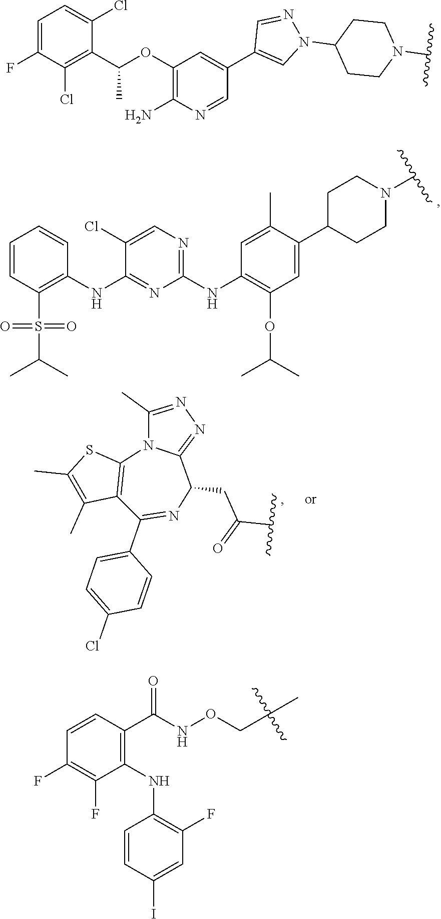 [SMILES: *N1CCC(n2cc(-c3cnc(N)c(O[C@H](C)c4c(Cl)ccc(F)c4Cl)c3)cn2)CC1 ; *N1CCC(c2cc(OC(C)C)c(Nc3ncc(Cl)c(Nc4ccccc4S(=O)(=O)C(C)C)n3)cc2C)CC1 ; *C(=O)C[C@@H]1N=C(c2ccc(Cl)cc2)c2c(sc(C)c2C)-n2c(C)nnc21 ; *CONC(=O)c1ccc(F)c(F)c1Nc1ccc(I)cc1F]

wherein

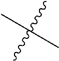

indicates the point of attachment to the L₁ group.

Various linkers may be used to link ULB and PB. In some embodiments, L₁ has the structure of formula (VI):

—Y₁—Y₂—Y₃—Y₄— (VII)

wherein Y₁-Y₄ are independently selected from
absent, —C(O)—, —O—, —OC(O)—, —NR^{a}, —N(R^{a})C(O)—, —(C(R^{a})(R^{a}))₁₋₁₂— (e.g., —(C(R^{a})(R^{a}))₁₋₁₀—, —(C(R^{a})(R^{a}))₁₋₈—, —(C(R^{a})(R^{a}))₁₋₆—, —(C(R^{a})(R^{a}))₁₋₄—, —(C(R^{a})(R^{a}))₁₋₂—) —(C(R^{a})(R^{a})C(R^{a})(R^{a})O)₁₋₁₂— (e.g., —(C(R^{a})(R^{a})C(R^{a})(R^{a})O)₁₋₁₀—, —(C(R^{a})(R^{a})C(R^{a})(R^{a})O)₁₋₈—, —(C(R^{a})(R^{a})C(R^{a})(R^{a})O)₁₋₆—, —(C(R^{a})(R^{a})C(R^{a})(R^{a})O)₁₋₄—, —(C(R^{a})(R^{a})C(R^{a})(R^{a})O)₁₋₂—), and —S—S—; and
R^{a} is independently selected at each occurrence from hydrogen and alkyl (e.g., C₁-C₄ alkyl). In some embodiments, Y₄ is —(C(R^{a})(R^{a}))₁₋₁₂— or —(C(R^{a})(R^{a})C(R^{a})(R^{a})O)₁₋₁₂—. In certain aspects, the compound may have the structure of formula (VIIa), (VIIb), (VIIc), (VIId), (VIIf), (VIIg), (VIIh), (VIIi), (VIIj), (VIIk), (VIIl), (VIIm), or (VIIn):

PB—NH—(CH₂)₁₋₁₀—NH—C(O)—ULB-L₂-TG (VIIa)

PB—(CH₂)₁₋₁₀—NH—C(O)—(CH₂)₁₋₁₀—ULB-L₂-TG (VIIb)

PB—(CH₂)₁₋₁₀—NH—(CH₂)₁₋₁₀—ULB-L₂-TG (VIIc)

PB—NH—(CH₂)₁₋₁₀—NH—C(O)—ULB-L₂-TG (VIId)

PB—C(O)—(CH₂)₁₋₁₀—ULB-L₂-TG (VIIe)

PB—NH—(CH₂)₁₋₁₀—ULB-L₂-TG (VIIg)

PB—NH—(CH₂CH₂O)₁₋₁₀—NH—C(O)—ULB-L₂-TG (VIIh)

PB—(CH₂CH₂O)₁₋₁₀—NH—C(O)—(CH₂CH₂O)₁₋₁₀—ULB-L₂-TG (VIIi)

PB—(CH₂CH₂O)₁₋₁₀—NH—(CH₂CH₂O)₁₋₁₀—ULB-L₂-TG (VIIj)

PB—NH—(CH₂CH₂O)₁₋₁₀—NH—C(O)—ULB-L₂-TG (VIIk)

PB—C(O)—(CH₂CH₂O)₁₋₁₀—ULB-L₂-TG (VIIl)

PB—NH—(CH₂CH₂O)₁₋₁₀—ULB-L₂-TG (VIIm)

PB—(CH₂)₁₋₁₀—C(O)—NH—(CH₂)₁₋₁₀—ULB-L₂-TG (VIIn)

In some embodiments, the compound is not a PROTAC. The present disclosure also embraces compounds without the protein binding conjugation moiety. As shown herein, targetting group conjagation, and particularly folate conjugation to certain ULB moieties (e.g., IMiD base ULBs) may also specifically promote degradation of cancer cells due to the higher expression of moieties on the cancerous membranes as compared to healthy cellular membranes (e.g., FOLR1). In some embodiments, the compounds of the present disclosure have the structure:

ULB-L₂-TG

wherein L₂, and TG may be as described herein and ULB is conjgated to L₂ as described herein (but is not conjugated to L1-PB).

Exemplary compounds of the present disclosure include Compound 1 (Folate-ARV-771) or Compound 2 (Folate-MS432) or Compound 3 (Folate-MS99) or Compound 4 (FA-S2-MS4048):

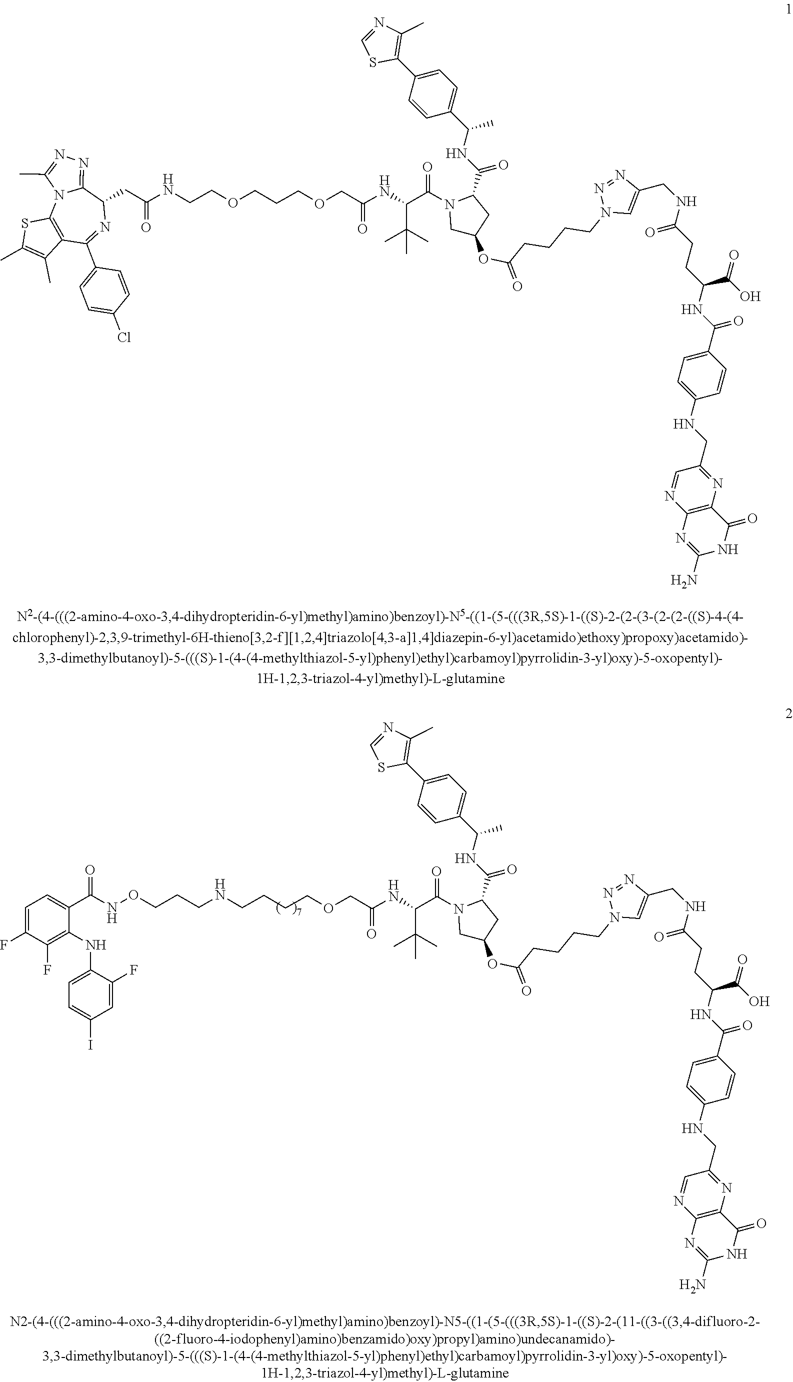

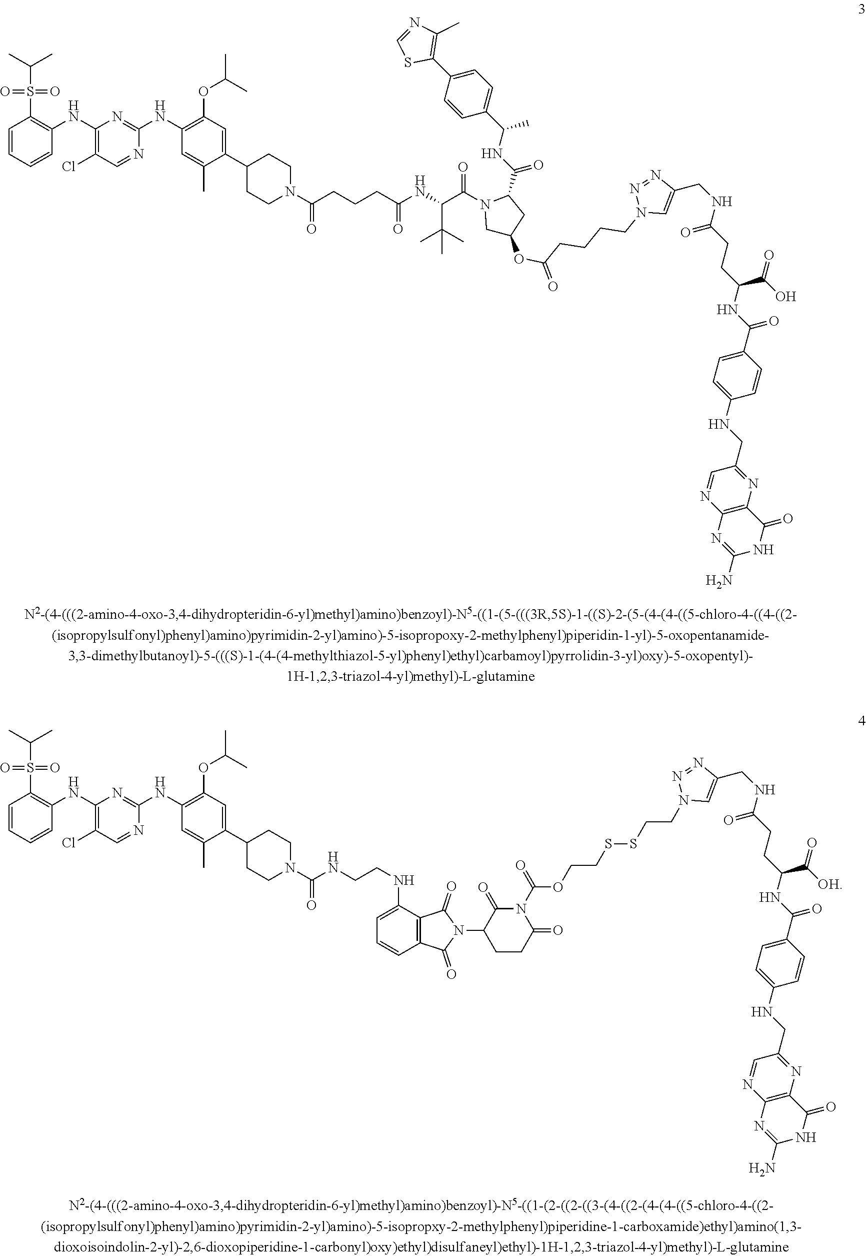

It will be understood that any for any discrepancy between the IUPAC name and compound structure above, both will be considered part of the disclosure.

Pharmaceutical compositions are also provided which comprise a compound of the present disclosure (e.g., Compounds having the structure of (I), (III), (IV), (V), (VI), (VII), Compound 1, Compound 2, Compound 3, Compound 4, pharmaceutically acceptable salts of any of the foregoing) and one or more pharmaceutically acceptable excipients, carriers, or diluents.

The disclosure includes methods for degrading a protein of interest comprising contacting the protein of interest with a compound as disclosed herein (e.g., Compounds having the structure of (I), (III), (IV), (V), (VI), (VII), Compound 1, Compound 2, Compound 3, Compound 4, pharmaceutically acceptable salts of any of the foregoing) and activating the compound through hydrolysis to cleave the targeting group from the ubiquitin ligase binding group and increase binding affinity of the compound for ubiquitin ligase.

Methods for reducing the proliferation or survival of a neoplastic cell are also provided, the method comprising contacting the cell with a compound as described herein (e.g., Compounds having the structure of (I), (III), (IV), (V), (VI), (VII), Compound 1, Compound 2, Compound 3, Compound 4, pharmaceutically acceptable salts of any of the foregoing),

wherein upon contact with said cell, the targeting group is cleaved from the ubiquitin ligase binding group and cell degradation occurs. In some embodiment, the neoplastic cell expresses FOLR1 and said targeting moiety is a folate derivative (e.g., having the structure of formula (II)). In some embodiments, the neoplastic cell is a prostate cancer cell, a breast cancer cell (e.g., triple negative breast cancer cell), a myeloma cell, a lymphoma cell (e.g., analplaastic large cell lymphoma) ovarian cancer cell, cervical cancer cell, or lung cancer cell (e.g., NSCLC).

The present disclosure also provides for methods for the treatment or prophylaxis of a proliferative disease in a subject in need thereof comprising administering a compound as disclosed herein (e.g., Compounds having the structure of (I), (III), (IV), (V), (VI), (VII), Compound 1, Compound 2, Compound 3, Compound 4, pharmaceutically acceptable salts of any of the foregoing) or a pharmaceutical composition as provided herein to said subject. In certain implementations, the proliferative disease is cancer. In some embodiments, the proliferative disease is a cancer selected from prostate cancer, a breast cancer (e.g., triple negative breast cancer), multiple myeloma, lymphoma (e.g., analplaastic large cell lymphoma), ovarian cancer, cervical cancer, and lung cancer (e.g., NSCLC).

### BRIEF DESCRIPTION OF FIGURES

FIG. 1 (FIGS. 1A-1F) illustrates a mechanism for how targeting group-conjugation delivers folate-PROTAC into cancer cells for targeted degradation of protein of interest. FIG. 1A is a schematic representation of the folate-PROTAC strategy. Following the FOLR1-mediated entrance into cancer cells, the folate group (light grey triangle) is cleaved by endogenous hydrolase, releasing the active PROTAC to degrade the protein of interest (POI). FIG. 1B is a schematic illustration of the activation of Folate-ARV-771 by endogenous hydrolase. FIG. 1C shows the western blot analysis of BRDs protein levels from HeLa cells treated with indicated doses of ARV-771, Folate-ARV-771, or Folate-ARV771N for 12 hours. FIG. 1D shows the western blot analysis of BRDs protein levels from HFF-1 cells treated with indicated doses of ARV-771, Folate-ARV-771, or Folate-ARV771N for 12 hours FIG. 1E shows the cell viability of HeLa cells after treatment with ARV-771, Folate-ARV-771, or Compound 1 (Folate-ARV-771N) for 72 hours. FIG. 1F shows the cell viability of HFF-1 cells after treatment with ARV-771, Folate-ARV-771, or Compound 1 (Folate-ARV-771N) for 72 hours.

FIG. 2 (FIGS. 2A-2D) illustrates Folate-ARV-771 degradation of BRD proteins in FOLR1-expressing cancer cells. FIG. 2A shows the chemical structure of Folate-ARV-771N. FIG. 2B illustrates that Folate-ARV-771 degrades BRDs in a time-dependent manner in HeLa cells. HeLa cells were treated with 30 nM of ARV-771 or Folate-ARV-771 for indicated time, and then harvested for Western Blot analysis for BRDs. FIG. 2C shows Folate-ARV-771 degrades BRDs in dose-dependent manner in OVCAR8 cells. OVCAR8 cells were treated with indicated dose of ARV-771, Folate-ARV-771 or Folate-ARV-771N for 12 hours, and then harvested for Western Blot analysis for BRDs. FIG. 2D shows that Folate-ARV-771 degrades BRDs in a time-dependent manner in OVCAR8 cells. OVCAR8 cells were treated with 30 nM of ARV-771 or Folate-ARV-771 for indicated time, and then harvested for Western Blot analysis for BRDs.

FIG. 3 (FIGS. 3A-3D) demonstrates Folate-ARV-771 degradation of BRD proteins in a VHL-dependent manner. FIG. 3A shows that Free VHL ligand (Ac-VHL) blocked the effect of Folate-ARV-771 in degrading BRDs in HeLa cells. HeLa cells were treated with indicated dose of VHL ligand together with 10 nM of Folate-ARV-771 FOR 12 hours, and then harvested for Western Blot analysis for BRDs. FIG. 3B illustrates the cell viability of HeLa cells after treatment with Folate-ARV-771 for 72 hours, with or without co-treatment of VHL ligand (Ac-VHL). FIG. 3C shows that knockout of VHL abolished the effect of Folate-ARV-771 in degrading BRDs in HeLa cells. HeLa cells were infected with sgVHL virus and selected with puromycin for 72 hours. The selected cells were treated with indicated dose of Folate-ARV-771 for 12 hours, and then harvested for Western Blot analysis for BRDs. FIG. 3D illustrates that cell viability of HeLa-VHL^{+/+} or HeLa-VHL^{-/-} cells after treatment with Folate-ARV-771 for 72 hours. NS: not sensitive.

FIG. 4 (FIGS. 4A-4D). Folate-ARV-771 degrades BRD4 in a FOLR1-dependent manner. FIG. 4A shows the Western blot analysis of BRD4 level in HeLa cells after the treatment with indicated doses of free folic acid either with Foalte-ARV-771 or ARV-771 for 12 hours. FIG. 4B shows the Cell viability of HeLa cells treated with Folate-ARV-771 with or without free folic acid for 72 hours. FIG. 4C illustrates analysis on HeLa cells with or without knockdown of endogenous FOLR1 which were treated with Folate-ARV-771 for 2 hours and then the levels of BRD4 as determined by western blot. FIG. 4D shows the Western blot analysis of BRD4 level in FOLR1-negative breast epithelial cells (MCF10A) versus breast cancer cell lines with either high FOLR1 expression (ZR-75-1, SKB-BR-3 and AU565) or low FOLR1 expression levels (BT549 and MDA-MB231) after the treatment with Folate-ARV-771 for 12 hours.

FIG. 5 (FIGS. 5A-5E) shows Folate-ARV-771 degradation of BRD proteins in a FOLR1-dependent manner. FIG. 5A illustrates that endocytosis inhibitor MCD blocked the effect of Folate-ARV-771 in degrading BRDs in HeLa cells. HeLa cells were treated with indicated doses of MCD together with Folate-ARV-771 for 12 hours, and then harvested for Western Blot analysis for BRD4. FIG. 5B shows the western Blot analysis of FOLR1 in different breast cancer cell lines. FIG. 5C illustrates that Folate-ARV-771 degrades BRDs in a dose-dependent manner in T47D cells. T47D cells were treated with ARV-771, Folate-ARV-771, or Folate-ARV-771N for 12 hours, and then harvested for Western Blot analysis for BRDs. FIG. 5D shows the cell viability of T47D cells after treating with ARV-771, Folate-ARV-771, or Folate-ARV-771N for 72 hours. FIG. 5E illustrates that knockdown of FOLR1 abolished the effect of Folate-ARV-771 in degrading BRDs in T47D cells. T47D cells were infected with shFORL1 virus and selected with puromycin for 72 hours. The selected cells were treated with indicated dose of Folate-ARV-771 for 2 hours, and then harvested for Western Blot analysis.

FIG. 6 (FIGS. 6A-6D) demonstrates Folate-MS432 degradation of MEK1/2 in a FOLR1-dependent manner. FIG. 6A shows a schematic illustration of the activation of Folate-MS432 by endogenous hydrolase. FIG. 6B illustrates western blot analysis of MEK1 and MEK2 levels in HT-29 cells treated with indicated doses of MS432, Folate-MS432, or Folate-MS432N for 12 hours. FIG. 6C is western blot analysis of MEK1 and MEK2 levels in HT-29 cells after the co-treatment with free folic acid and Folate-MS432 for 12 hours. FIG. 6D shows the cell viability of HT-29 cells after treatment with MS432, Folate-MS432, or Folate-MS432N for 72 hours.

FIG. 7 (FIGS. 7A-7H) shows Folate-MS432 degradation of MEK1/2 proteins in a FOLR1-dependent manner. FIG. 7A illustrates the chemical structure of Folate-MS432N. FIGS. 7B and 7C show that Folate-MS432 degrades MEK1/2 in a dose-dependent manner in HeLa and SK-MEL-28 cells. HeLa and SK-MEL-28 cells were treated with indicated dose of MS432, Folate-MS432, or Folate-MS432N for 12 hours, and then harvested for Western Blot analysis for MEK1/2. FIGS. 7D and 7E illustrate that Folate-MS432 degrades MEK1/2 in a time-dependent manner in HT-29 cells and SK-MEL-28 cells. HT-29 cells and SK-MEL-28 cells were treated with 0.3 μM of MS432 or Folate-MS432 for indicated time, and then harvested for Western Blot analysis for MEK1/2. FIG. 7F shows that free folic acid antagonized the effect of Folate-MS432 in degrading MEK1/2 in SK-MEL-28 cells. SK-MEL-28 cells were treated with indicated dose of folic acid together with 0.3 μM of Folate-MS432 for 12 hours, and then harvested for Western Blot analysis for MEK1/2. FIG. 7G shows that free VHL ligand (Ac-VHL) blocked the effect of Folate-MS432 in degrading MEK1/2 in HT-29 cells. HT-29 cells were treated with indicated dose of VHL ligand together with 0.3 μM of Folate-MS432 for 12 hours, and then harvested for Western Blot analysis for MEK1/2. FIG. 7H shows the cell viability of SK-MEL-28 cells after treating with MS432, Folate-MS432, or Folate-MS432N for 72 hours.

FIG. 8 (FIGS. 8A-8G) shows Folate-MS99 degradation of ALK fusion proteins in a FOLR1-dependent manner. FIG. 8A shows a schematic diagram to show the ALK fusion proteins in cancer cells. FIG. 8B shows the chemical structure of Folate-MS99N. FIG. 8C illustrates that Folate-MS99 degraded the NPM-ALK fusion protein in a time-dependent manner in SU-DHL-1 cells. SU-DHL-1 cells were treated with 0.3 μM of MS99, or Folate-MS99 for indicated hours, and then harvested for Western Blot analysis for NPM-ALK. FIGS. 8D and 8E shows that Folate-MS99 degraded the EML4-ALK fusion protein in a dose-dependent manner in NCI-H2228 and NCI-H3122 non-small cell lung cancer (NSCLC) cells. NCI-H2228 and NCI-H3122 cells were treated with indicated doses of MS99, Folate-MS99, or Folate-MS99N for 12 hours, and then harvested for Western Blot analysis for EML4-ALK. FIGS. 8F and 8G show that free folic acid antagonized the effect of Folate-MS99 in degrading the EML4-ALK fusion protein in NCI-H2228 and NCI-H3122 cells. NCI-H2228 and NCI-H3122 cells were treated with indicated dose of folic acid together with 0.3 μM of Folate-MS99 for 12 hours, and then harvested for Western Blot analysis for EML4-ALK.

FIG. 9 (FIGS. 9A-9D) illustrates Folate-MS99 degradation of ALK fusion proteins in a FOLR1-dependent manner. FIG. 9A provides a schematic illustration of the activation of Folate-MS99 by endogenous hydrolase. FIG. 9B shows western blot analysis of NPM-ALK fusion protein level from SU-DHL-1 cells treated with indicated doses of MS99, Folate-MS99, or Folate-MS99N for 12 hours. FIG. 9C shows western blot analysis of NPM-ALK fusion protein from HT-29 cells after the co-treatment with free folic acid and Folate-MS99 for 12 hours. FIG. 9D shows the cell viability of SU-DHL-1 cells after treatment with MS99, Folate-MS99, or Folate-MS99N for 72 hours.

FIG. 10 is a schematic illustrating PROTAC activation via intracellular glutathione (GSH) to reduce disulfide linkers. Upon binding FOLR1 on the cell membrane (1), folate pomalidomide or folate-conjugated IMiD-based PROTACs are transported into cells, and the active pomalidomide or PROTACs are released after the reduction by endogenous glutathione (2). The active pomalidomide or PROTACs recruit endogenous CRBN E3 ligase, leading to polyubiquitination and subsequent degradation of IKZFs or the proteins of interest (POIs) by the ubiquitin-proteasome system (UPS) (3).

FIG. 11 (FIGS. 11A-11F) illustrate the relase of pomalidomide from FA-S2-POMA in vitro. FIG. 11A is a schematic diagram shown the chemical structures of folate-S2-pomalidomide (FA-S2-POMA) and the negative control used (folate-C2-pomalidomide, FA-C2-POMA). FA-S2 POMA is cleaved after reduction by GSH, releasing a folate-derivative (Folate-SH) and pomalidomide derivative (poma-SH). The pomalidomide derivative then releases pomalidomide and 1,3-oxathiolan-2-one via a spontaneous intramolecular cyclization. FIG. 11B shows the measured release of pomalidomide from FA-S2-POMA in PBS at 37° C. after incubation with DTT (2 equiv), measured by HPLC. FIG. 11C provides the HPLC analysis of FA-S2-POMA for 5 and 20 minutes. FIG. 11D shows the illustrates the HPLC analysis of the negative control FA-C2-POMA after incubation with DTT (2 equiv) at 37° C. in PBS for 5 and 20 min. FIG. 11E illustrates that no pomalidomide was released from FA-C2-POMA following incubation with DTT from the HPLC analysis provided in FIG. 11D. FIG. 11F shows that reduction of FA-S2-POMA with GSH leads to the release of pomalidomide, which competes with biotinylated pomalidomide (Biotin-POMA) for binding with Flag-tagged cereblon (CRBN). Cell lysis derived from HEK293T cells were incubated with biotin or Biotin-POMA, with or without pomalidomide, FA-S2-POMA, or FA-C2-POMA for 1 hour, and with or without pretreated GSH (+: 1 mM, ++: 5 mM).

FIG. 12 (FIGS. 12A-12C) illustrates that FA-S2-POMA degrades IKZF3 ande IKZF1. FIG. 12A shows the western blotting of FOLR1 and IKZF3 in THP-1, MM.1S and SU-DHL-1 cells. FIG. 12B shows the western blotting of IKZF1 in SU-DHL-1 cells treated with the indicated concentrations of pomalidomide (POMA) or FA-S2-POMA for 12 hours. FIG. 12C shows the western blotting of IKZF3 in MM.1S cells treated with the indicted concentrations of pomalidomide (POMA) for 12 hours, with or without co-treatment of S-acetyl-glutathione (S—Ac-GSH, 1 mM) for 12 hours.

FIG. 13 (FIGS. 13A-13F) illustrates that FA-S2-POMA degrades IKZF3 in FOLR1-positive cancer cells in a concentration and time dependent manner. FIG. 13A shows the western blots of IKZF3 in MM.1S cells treated with the indicated concentrations of pomalidomide, FA-S2-POMA, or FA-C2-POMA for 12 hours. FIG. 13B provides the western blotting of IKZF3 in MM.1S cells treated with 0.1 μM pomalidomide or 1 μM FA-S2-POMA for the indicated times. FIG. 13C is the western blotting of IKZF3 in THP-1 cells treated with the indicated concentrations of pomalidomide or FA-S2-POMA for 12 hours. FIG. 13D provides the cell viability of MM.1S after treatment with the indicated concentrations of pomalidomide, FA-S2-POMA, or FA-C2-POMA for 72 hours. FIG. 13E is the western blots of IKZF3 in MM.1S cells treated with the indicated concentrations of FA-S2-POMA for 12 hours, with or without pre-treatment with S-acetyl-glutathione (S—Ac-GSH, 1 mM) for 12 hours. FIG. 13F shows the cell viability of MM.1S cells after treatment with the indicated concentrations of FA-S2-POMA for 12 hours, with or without pre-treatment with S-acetyl-glutathione (S—Ac-GSH, 1 mM) for 12 hours.

FIG. 14 (FIGS. 14A-14I) shows that FA-S2-POMA degrades IKZF3 in a FOLR1-, CRBN-, and proteasome-dependent manner. FIG. 14A is the western blotting of IKZF3 in MM.1S cells treated with the indicated concentrations of FA-S2-POMA, with or without the presence of 2.5 mM of folic acid. FIG. 14B is the western blotting of IKZF3 in MM.1S cells treated with 0.1 μM of pomalidomide with or without treatment of 2.5 mM of folic acid for 12 hours. FIG. 14C shows the cell viability of MM.1S cells after treatment with the indicated concentrations of FA-S2-POMA for 72 hours, with or without the presence of folic acid. FIG. 14D shows the western blotting of IKZF3 in MM.2S cells treated with the indicated concentration of the endocytosis inhibitor Pitstop 2 for 12 hours, followed by the treatment with 1 μM of FA-S2 POMA for another 12 hours. FIG. 14E shows the western blotting of IKZF1 in SU-DHL-1 cells treated with the indicated concentration of the endocytosis inhibitor MβCD for 12 hours, followed by the treatment with 1 μM of FA-S2-POMA for another 12 hours. FIG. 14F shows the western blotting of IKZF3 in MM.1S-CRBN-WT or MM.1S-CRBN-KO cells treated with the indicated concentrations of pomalidomide, FA-S2-POMA, or FA-C2-POMA for 12 hours. FIGS. 14G-H show the western blotting of IKZF3 in MM.1S-CRBN^{+/+} or MM.1S-CRBN^{-/-} cells treated with the indicated concentrations of pomalidomide or FA-S2-POMA for 12 hours. FIG. 14I shows the western blotting of IKZF3 in MM.2S cells treated with 1 μM of FA-S2-POMA for 12 hours, with or without the presence of proteasome inhibitors (10 0 μM of MG132 or 1 μM of MLN4924).

FIG. 15 illustrates the chemical structures of FA-S2-MS4048 (Compound 4) and its negative control, FA-C2-MS4048.

FIG. 16 (FIGS. 16A-16D) illustrate the release of the PROTAC MS4048 from FA-S2-MS4048, but not from Folate-C2-MS4048. FIG. 16A provides HPLC analysis of FA-S2-MS4048 after incubation with DTT (10 equiv) at 37° C. in PBS for 0.5, 1, and 2 hours. FIG. 16 shows the release of MS4048 from FA-S2-MS4048 after incubation with DTT (10 equiv) at 37° C. in PBS for the indicated time. FIG. 16C shows the HPLC analysis of FA-C2-MS4048 after incubation with DTT (10 equiv) at 37° C. in PBS for 0.5, 1, and 2 hours. FIG. 16D shows that there was no release of MS4048 from FA-C2-MS4048 after incubation with DTT (10 equiv) at 37° C. in PBS for the indicated time.

FIG. 17 (FIGS. 17A-17L) illustrates that FA-S2-MS4048 degrades NPM-ALK, an ALK fusion protein, in a concentration and time dependent manner. FIG. 17A is the western blotting of NPM-ALK in SU-DHL-1 cells treated with the indicated concentrations of NS4048, FA-S2-MS4048 or FA-C2-MS4048 for 12 hours. FIGS. 17B-E are the western blotting of EML4-ALK fusion protein in two NSCLC cell lines, NCI-H2228 and NCI-H3122, treated with the indicated concentration of MS4048, FA-S2-MS4048, or FA-C2-MS4048 for 12 hours. FIG. 17F shows the western blotting of NPM-ALK in SU-DHL-1 cells treated with 03 μM of MS4048 or 3 μM of FA-S2-MS4048 for the indicated times. FIG. 17G illustrates the cell viability of SU-DHL-1 cells after treatment with the indicated concentrations of MS4048, FA-S2-MS4048 or FA-C2-MS4048 for 72 hours. FIG. 17H shows the western blotting of NPM-ALK in SU-DHL-1 cells treated with the indicated concentrations of MS4048, FA-S2-MS4048, or FA-C2-MS4048 for 72 hours. FIG. 17I shows the western blotting of NPM-ALK fusion protein in SU-DHL-1 cells treated with indicated concentrations of MS4048, with or without co-treatment of S—Ac-GSH (1 mM) for 12 hours. FIGS. 17J-K provide the western blotting of EML4-ALK fusion protein in NCI-H2228 and NCI-H3122 cells treated with the indicated concentrations of FA-S2-MS4048, with or without treatment of S—Ac-GSH (mM) for 12 hours. FIG. 17L provides cell viability of SU-DHL-1 cells after treatment with the indicated concentrations of FA-S2-MS4048 for 72 hours, with or without pretreatment with S—Ac-GSH (1 mM) for 12 hours.

FIG. 18 (FIGS. 18A-18G) illustrate that FA-S2-MS4048 degrades ALK fusion proteins in a FOLR1, CRBN, and proteasome dependent manner. FIG. 18A provides the wester blotting of NPM-ALK in SU-DHL-1 cells treated with MS4048 (0.3 μM) or FA-S2-MS4048 (3 μM), with or without the presence of 2.5 mM of folic acid. FIG. 18B illustrates the cell viability of SU-DHL-1 cells after treatment with the indicated concentrations of FA-S2-MS4048 for 72 hours, with or without the presence of 2.5 mM of folic acid. FIG. 18C shows the western blotting of NPM-ALK in SU-DHL-1 cells with knockdown of FOLR1 and treatment with FA-S2-MS4048 (3 μM) for 12 hours. FIG. 18D shows the western blotting of NPM-ALK in SU-DHL-1 cells with CRBN-WT or CRBN-KO cells and treated with FA-S2-MS4048 (3 μM) for 12 hours. FIG. 18E provides western blotting of NPM-ALK in SU-DHL-1 cells treated with 3 μM of FA-S2-MS4048 for 12 hours, with or without the presence of 10 μM of MG132 or 1 μM of MLN4924.

### DETAILED DESCRIPTION

Detailed embodiments of the present disclosure are disclosed herein; however, it is to be understood that the disclosed embodiments are merely illustrative and may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. Moreover, all values that fall within these ranges, as well as the upper or lower limits of a range of values, are also contemplated by the present application. For example, the exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that sum of all weight percentages does not exceed 100% unless otherwise specified.

By “consist essentially” it is meant that the ingredients include only the listed components along with the normal impurities present in commercial materials and with any other additives present at levels which do not affect operation, for instance at levels less than 5% by weight or less than 1% or even 0.5% by weight.

Typically, alkyl groups described herein refer to a branched or straight-chain monovalent saturated aliphatic hydrocarbon radical of 1-30 carbon atoms (e.g., 1-16 carbon atoms, 6-20 carbon atoms, 8-16 carbon atoms, or 4-18 carbon atoms, 4-12 carbon atoms, 1-4 carbon atoms, 1-8 carbon atoms). In some embodiments, the alkyl group may be substituted with 1, 2, 3, or 4 substituent groups as defined herein. Alkyl groups may have from 1-26 carbon atoms. In other embodiments, alkyl groups will have from 6-18 or from 1-8 or from 1-6 or from 1-4 or from 1-3 carbon atoms, including for example, embodiments having one, two, three, four, five, six, seven, eight, nine, or ten carbon atoms. Any alkyl group may be substituted or unsubstituted. Examples of alkyl groups include methyl, ethyl, propyl, butyl, pentyl, hexyl, heptyl, octyl, nonyl, decyl, undecyl, and dodecyl groups. Heteroalkyl groups may refer to branched or straight-chain monovalent saturated aliphatic hydrocarbon radicals with one or more heteroatoms (e.g., N, O, S, etc.) in the carbon chain. Heteroalkyl groups may have 1-30 carbon atoms (e.g., 1-16 carbon atoms, 6-20 carbon atoms, 8-16 carbon atoms, or 4-18 carbon atoms, 4-12 carbon atoms, etc.). In some embodiments, the heteroalkyl group may be substituted with 1, 2, 3, or 4 substituent groups as defined herein. Heteroalkyl groups may have from 1-26 carbon atoms. In other embodiments, heteroalkyl groups will have from 6-18 or from 1-8 or from 1-6 or from 1-4 or from 1-3 carbon atoms, including for example, embodiments having one, two, three, four, five, six, seven, eight, nine, or ten carbon atoms. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O—; and “alkoyl” which, as used herein, refers to alkyl-CO—. Alkoxy substituent groups or alkoxy-containing substituent groups may be substituted by, for example, one or more alkyl groups.

Aryl groups may be aromatic mono- or polycyclic radicals of 6 to 12 carbon atoms having at least one aromatic ring. Examples of such groups include, but are not limited to, phenyl, naphthyl, 1,2,3,4-tetrahydronaphthalyl, 1,2-dihydronaphthalyl, indanyl, and 1H-indenyl. Aryl groups as used herein may be substituted or unsubstitued. Typically, heteroaryls include mono- or polycyclic radical of 5 to 12 atoms having at least one aromatic ring containing one, two, or three ring heteroatoms selected from N, O, and S, with the remaining ring atoms being C. One or more ring carbon atoms of the heteroaryl group may be replaced with a carbonyl group. Examples of heteroaryl groups are pyridyl, triazolyl, benzooxazolyl, benzoimidazolyl, and benzothiazolyl.

Linking groups, when present, are typically referred to as divalent hydrocarbon groups (e.g., alkylene, heteroalkylene, alkynylene, alkeneylene, heteroalkynylene, heteroalkeneylene, arylene, heteroarylene), where each radical position is the point of attachment. Alkylene groups may refer to a straight or branched chain divalent hydrocarbon radical having from one to ten carbon atoms, optionally substituted with substituents selected from the group which includes lower alkyl (e.g., C₁-C₄, etc.), lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen and lower perfluoroalkyl, multiple degrees of substitution being allowed. Examples of alkylene as used herein include, but are not limited to, methylene, ethylene, n-propylene, n-butylene, and the like. Alkylene groups may be saturated or unsaturated. Heteroalkylene groups may be alkylene groups comprising one or more heteroatoms (e.g., N, S, O) in the carbon chain. Cycloalkylene groups may be divalent hydrocarbons comprising one or more saturated or unsaturated cycloalkyl groups. The two points of attachment on cycloalkylene groups may be at two points in the ring, for example, at vicinal positions, germinal positions. Cycloalkylene groups may be divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic unsaturated ring systems that respectively include at least one double bond and at least one triple bond. Cycloalkylene, Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms.

It will be understood that any divalent linking moiety with multiple points of attachment (each typically indicated with “—”) may be attached to the specified moieties in either direction to the extent permitted by valency, unless otherwise indicated (e.g., by indicating the attachments). For example, a linking moiety having the structure —X₁—X₂— may be used to link two portions of a compound in the —X₁—X₂— orientation or in the —X₂—X₁— orientation. However, PB-L₁-ULB—X₁—X₂-TG does not include PB-L₁-ULB—X₂—X₁-TG unless otherwise indicated.

The ring systems of the saturated or unsaturated cycloalkyl, aryl, heterocylcoalkyl, heteroaryl, cycloalkylene, cycloalkenylene and cycloalkynylene groups may be composed of one ring or two or more rings which may be joined together in a fused, bridged or spiro-connected fashion. Heteocyclo and Heterocyclene groups may be divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur. Aryl and Arylene groups may be monocyclic or polycyclic, in certain embodiments monocyclic, aromatic group, in some embodiments having from 5 to about 20 carbon atoms and at least one aromatic ring, in another embodiment 5 to 12 carbons. Arylene groups include, but are not limited to, 1,2-, 1,3- and 1,4-phenylene. Heteroarylene groups are typically divalent monocyclic or multicyclic aromatic ring systems, in one embodiment of about 5 to about 15 atoms in the ring(s), where one or more, in certain embodiments 1 to 3, of the atoms in the ring system is a heteroatom, that is, an element other than carbon, including but not limited to, nitrogen, oxygen or sulfur.

The term “substituent” refers to a group “substituted” on, e.g., an alkyl, at any atom of that group, replacing one or more hydrogen atoms therein. In some aspects, the substituent(s) on a group are independently any one single, or any combination of two or more of the permissible atoms or groups of atoms delineated for that substituent. In another aspect, a substituent may itself be substituted with any one of the substituents described herein.

A substituted hydrocarbon group may have as a substituent one or more hydrocarbon radicals, substituted hydrocarbon radicals, or may comprise one or more heteroatoms. Examples of substituted hydrocarbon radicals include, without limitation, heterocycles, such as heteroaryls. Unless otherwise specified, a hydrocarbon substituted with one or more heteroatoms will comprise from 1-20 heteroatoms. In other embodiments, a hydrocarbon substituted with one or more heteroatoms will comprise from 1-12 or from 1-8 or from 1-6 or from 1-4 or from 1-3 or from 1-2 heteroatoms. Examples of heteroatoms include, but are not limited to, oxygen, nitrogen, sulfur, phosphorous, halogen (e.g., F, Cl, Br, I, etc.), boron, silicon, etc. In some embodiments, heteroatoms will be selected from the group consisting of oxygen, nitrogen, sulfur, phosphorous, and halogen (e.g., F, Cl, Br, I, etc.). In some embodiments, a heteroatom or group may substitute a carbon. In some embodiments, a heteroatom or group may substitute a hydrogen. In some embodiments, a substituted hydrocarbon may comprise one or more heteroatoms in the backbone or chain of the molecule (e.g., interposed between two carbon atoms, as in “oxa”). In some embodiments, a substituted hydrocarbon may comprise one or more heteroatoms pendant from the backbone or chain of the molecule (e.g., covalently bound to a carbon atom in the chain or backbone, as in “oxo”).

In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent (e.g., a hydrocarbon), the group may be referred to as “R-substituted.” Where a moiety is R-substituted, the moiety is substituted with at least one R substituent and each R substituent is optionally different.

Unless otherwise noted, all groups described herein (e.g., alkyl, cycloalkyl, heteroalkyl, heterocycloalkyl, aryl, heteroaryl, alkylene, heteroalkylene, cylcoalkylene, arylene, heteroaryelene, heterocycloalkylene) may optionally contain one or more common substituents, to the extent permitted by valency. Common substituents include halogen (e.g., F, Cl, etc.), C₁₋₁₂ straight chain or branched chain alkyl, C₂₋₁₂ alkenyl, C₂₋₁₂ alkynyl, C₃₋₁₂ cycloalkyl, C₆₋₁₂ aryl, C₃₋₁₂ heteroaryl, C₃₋₁₂ heterocyclyl, C₁₋₁₂ alkylsulfonyl, nitro, cyano, —COOR, —C(O)NRR′, —OR, —SR, —NRR′, and oxo, such as mono- or di- or tri-substitutions with moieties such as halogen, fluoroalkyl, perfluoroalkyl, perfluroalkoxy, trifluoromethoxy, chlorine, bromine, fluorine, methyl, methoxy, pyridyl, furyl, triazyl, piperazinyl, pyrazoyl, imidazoyl, and the like, each optionally containing one or more heteroatoms such as halo, N, O, S, and P. R and R′ are independently hydrogen, C₁₋₁₂ alkyl, C₁₋₁₂haloalkyl, C₂₋₁₂ alkenyl, C₂₋₁₂ alkynyl, C₃₋₁₂ cycloalkyl, C₄₋₂₄ cycloalkylalkyl, C₆₋₁₂ aryl, C₇₋₂₄ aralkyl, C₃₋₁₂ heterocyclyl, C₃₋₂₄ heterocyclylalkyl, C₃₋₁₂ heteroaryl, or C₄₋₂₄ heteroarylalkyl. Further, as used herein, the phrase optionally substituted indicates the designated hydrocarbon group may be unsubstituted (e.g., substituted with H) or substituted. Typically, substituted hydrocarbons are hydrocarbons with a hydrogen atom removed and replaced by a substituent (e.g., a common substituent). It is understood by one of ordinary skill in the chemistry art that substitution at a given atom is limited by valency. The use of a substituent (radical) prefix names such as alkyl without the modifier optionally substituted or substituted is understood to mean that the particular substituent is unsubstituted. However, the use of haloalkyl without the modifier optionally substituted or substituted is still understood to mean an alkyl group, in which at least one hydrogen atom is replaced by halo.

Compounds provided herein (e.g., Compounds having the structure of (I), (III), (IV), (V), (VI), (VII), Compound 1, Compound 2, Compound 3, Compound 4, etc.) can have one or more asymmetric carbon atoms and can exist in the form of optically pure enantiomers, mixtures of enantiomers such as racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as chiral chromatography and separation methods based thereon. The appropriate technique and/or method for separating an enantiomer of a compound described herein from a racemic mixture can be readily determined by those of skill in the art. “Racemate” or “racemic mixture” means a mixture containing two enantiomers, wherein such mixtures exhibit no optical activity; i.e., they do not rotate the plane of polarized light. “Geometric isomer” means isomers that differ in the orientation of substituent atoms (e.g., to a carbon-carbon double bond, to a cycloalkyl ring, to a bridged bicyclic system, etc.). Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds disclosed herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99%, or 99.9%) by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer.

Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

It will be understood that the description of compounds herein is limited by principles of chemical bonding known to those skilled in the art. Accordingly, where a group may be substituted by one or more of a number of substituents, such substitutions are selected so as to comply with principles of chemical bonding with regard to valencies, etc., and to give compounds which are not inherently unstable. For example, any carbon atom will be bonded to two, three, or four other atoms, consistent with the four valence electrons of carbon. Additionally, when a structure has less than the required number of functional groups indicated, those carbon atoms without an indicated functional group are bonded to the requisite number of hydrogen atoms to satisfy the valency of that carbon.

The term “pharmaceutical composition,” as used herein, represents a composition containing a compound described herein (e.g., Compounds having the structure of (I), (III), (IV), (V), (VI), (VII), Compound 1, Compound 2, Compound 3, Compound 4, etc.) formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution free of particulate emboli and in a solvent system suitable for intravenous use); or in any other formulation described herein (see below). The pharmaceutical composition may comprise, for example, from 0.1% to 25% of the compounds of the present disclosure by weight of the composition.

Useful pharmaceutical carriers for the preparation of the compositions hereof, can be solids, liquids, or gases. Thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, and aerosols. The carrier can be selected from the various oils including those of petroleum, animal, vegetable or synthetic origin, e.g., peanut oil, soybean oil, mineral oil, and sesame oil. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution and rendering the solution sterile. Suitable pharmaceutical excipients include starch, cellulose, chitosan, talc, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions may be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin, which is hereby incorporated by reference in its entirety. Such compositions will, in any event, contain an effective amount of the active compound together with a suitable carrier so as to prepare the proper dosage form for administration to the recipient.

As used herein, the term “pharmaceutically acceptable salt” refers to salts of any of the compounds described herein that within the scope of sound medical judgment, are suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008, each of which are hereby incorporated by reference in their entirety. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecylsulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine.

As used herein, the term “subject” refers to any organism to which a composition in accordance with the disclosure may be administered, e.g., for experimental, diagnostic, prophylactic, and/or therapeutic purposes. In most embodiments, the subject is a human. Other subjects may include mammals such as mice, rats, rabbits, cats, dogs, non-human primates. The subject may be domesticated animals (e.g., cows, calves, sheep, goat, lambs, horses, poultry, foals, pigs, piglets, etc.), or animals in the family Muridae (e.g., rats, mice, etc.), or animals in the family Felidae. A subject may seek or be in need of treatment, require treatment, be receiving treatment, may be receiving treatment in the future, or a human or animal that is under care by a trained professional for a particular disease or condition (e.g., cancer, etc.).

Typically, a proliferative disease refers to the physiological condition in a subject characterized by unregulated cell growth such as cancer. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small cell lung cancer, non-small cell lung cancer (“NSCLC”), vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, as well as head and neck cancer. In yet other embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma, myeloproliferative diseases, large B cell lymphoma, and B cell Lymphoma.

The term “unit dosage form” refers to a physically discrete unit suitable as a unitary dosage for human subjects and other mammals, each unit containing a predetermined quantity of active material (e.g., a compound of the present disclosure) calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, gel cap, and syrup.

The term “effective amount” or “therapeutically effective amount” of an agent, as used herein (e.g., Compounds having the structure of (I), (III), (IV), (V), (VI), (VII), Compound 1, Compound 2, Compound 3, Compound 4, etc.), is that amount sufficient to effect beneficial or desired results, such as clinical results, and, as such, an “effective amount” depends upon the context in which it is being applied. In one embodiment, an effective amount is the amount of an irradiated compound described herein sufficient to affect the degradation of a protein of interest. In another embodiment, in the context of administering an agent that is an anticancer agent, an effective amount of an agent is, for example, an amount sufficient to achieve alleviation or amelioration or prevention or prophylaxis of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition (e.g., cancer, etc.); and remission (whether partial or total), whether detectable or undetectable, as compared to the response obtained without administration of the agent. In some embodiments, the effective amount assumes that more than 50% of the compounds administered release the photolabile group under irradiation conditions (e.g., more than 60%, more than 70%, more than 80%, more than 90%, more than 95%, more than 99%, 100%, etc.) to achieve the active compound capable of degrading a protein of interest.

The present targeting caged PROTAC typically cleave following cell membrane receptor mediated endocytosis of the compounds of the present disclosure. Following endocytosis, the targeting group is cleaved to allow ubiquitin recruitment and degradation of the protein of interest. Typically, cleavage does not occur extracellularly in the targeting group caged compounds of the present disclosure. Without wishing to be bound by theory, targeting group caged PROTACs afford the selective anticancer activity through cleavage occurring following compound entry into the cell. The present targeting group caged PROTAC comprising a targeting group that cleaves following endocytosis of the parent compound to result in a compound capable of ubiquitin recruitment to degrade the compound of interest only upon selective. Examples of moieties on the ubiquitin recruitment useful for installation of the targeting group include hydroxyl, amino, sulfhydryl, or carboxyl group of the ULB portion, and that when administered to a mammalian subject, cleaves within the cell via endogenous hydrolases to form the free hydroxyl, amino, sulfhydryl, or carboxyl group, respectively required for ubiquitin recruitment.

These compounds may have the structure of formula (I):

PB-L₁-ULB-L₂-TG (I)

wherein ULB is a ubiquitin ligase binding moiety;
L₁ is absent or a linker;
L₂ is absent or a linker;
PB is a protein binding moiety; and
TG is a targeting group that preferentially binds to a protein with increased expression in a neoplastic cell as compared to an otherwise identical healthy cell;
   wherein ubiquitin ligase binding potential of said compound is increased following cleavage between the ULB group and the TG group;
   or pharmaceutically acceptable salts thereof.

Various compounds capable of recruiting ubiquitin may be used in the compounds disclosed herein. Typically, the compound comprises a moiety derived from one of these structures wherein the indicated required groups (e.g., L₂, TG) are linked to the ULB structure through what is a hydrogen in the underivatized structure. In some embodiments, the derivatization of TG occurs at the position of ionic charge (e.g., at the —COO⁻ moiety of an anion such as folate). Typically, a “derivative” of a parent compound may comprise at least one distinction from its parent such as a bond in place of a hydrogen. Any derivitized moiety may also be substituted one or more times in any other hydrogen locations. For example, in some embodiments, the ULB moiety binds to an E3 ubiquitin ligase. The E3 ubiquitin ligase may comprise von Hippel Lindau (VHL) E3 ubiquitin ligase, β-Transducin Repeat Containing (β-TRCP) E3 Ubiquitin Protein Ligase, Mouse Double Minute 2 (Mdm2) E3 Ubiquitin Protein Ligase, or a Cereblon (CRBN) E3 Ubiquitin ligase.

The protein binding moiety may be chosen for a specific protein of interest (POI). Due to the chimeric structure of PROTACs, any compound which binds the POI may be used, such that the protein binding moiety of the compound may be linked (e.g., through a hydrogen position, etc.) to the other portions of the compound (e.g., L, ULB, TG, etc.) and retain some affinity for the protein of interest. Typically, the POI is degraded following irradiation of the photolabile group and activation of the ULB moiety. In certain embodiments, the protein binding moiety is a protein inhibitor, a protein modulator, or a protein activator. The protein binding moiety may be, for example, a tyrosine kinase inhibitor, a BRAF-mutant inhibitor, or a MEK inhibitor. In some embodiments, the protein binding moiety binds to one or more of Abelson Murine Leukemia (ABL) Proteins (e.g., c-ABL, etc.), Breakpoint Cluster Region Protein (BCR), BCR-ABL fusion proteins, Bromodomain and Extra Terminal Domain (BRD) Family proteins (e.g., BRD2, BRD3, BRDT, BRD4, etc.), anaplastic lymphoma kinase (ALK) protein, echinoderm microtubule-associated protein like, epidermal growth factor (EGFR) family of proteins, and echinoderm microtubule-associated protein like (EML)-ALK fusion proteins.

In certain embodiments, the protein binding moiety may be derived from crizotinib, certinib, PD0325901, or JQ1. For example, the protein binding moiety may be any of the following exemplary derivatives:

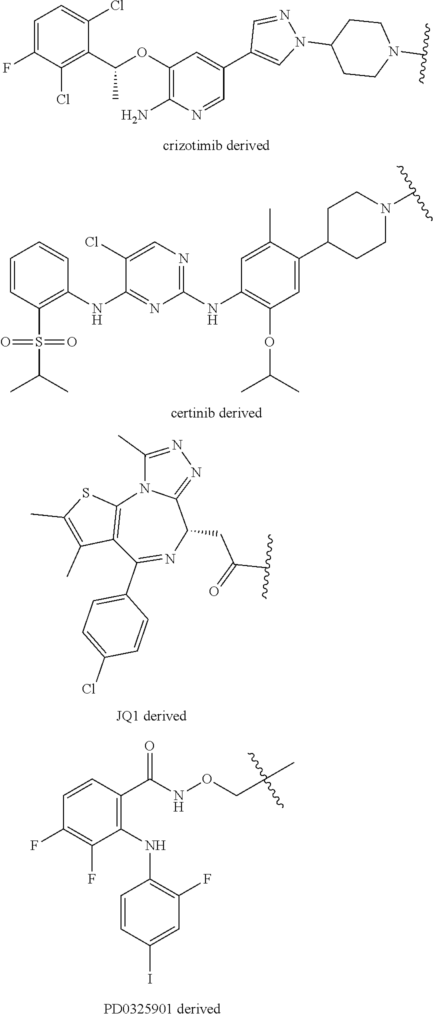 [SMILES: *N1CCC(n2cc(-c3cnc(N)c(O[C@H](C)c4c(Cl)ccc(F)c4Cl)c3)cn2)CC1 ; *N1CCC(c2cc(OC(C)C)c(Nc3ncc(Cl)c(Nc4ccccc4S(=O)(=O)C(C)C)n3)cc2C)CC1 ; *C(=O)C[C@@H]1N=C(c2ccc(Cl)cc2)c2c(sc(C)c2C)-n2c(C)nnc21 ; *CONC(=O)c1ccc(F)c(F)c1Nc1ccc(I)cc1F]

wherein

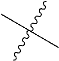

indicates the point of attachment to the L₁ group. In certain embodiments, the protein binding moiety may be derived from (e.g., linked through a hydrogen position of, etc.) Dasatinib, Imatinib, Saracatinib, Ponatinib, Nilotinib, Danusertib, AT9283, Degrasyn, Bafetinib, KW-2449, NVP-BHG712, DCC-2036, GZD824, GNF-2, PD173955, GNF-5, Bosutinib, Gefitinib, Erlotinib, Nivolumab, Sunitinib, Ruxolitinib, Tofacitinib, Lapatinib, Vandetanib, Sorafenib, Sunitinib, Axitinib, Nintedanib, Regorafenib, Pazopanib, Lenvatinib, Crizotinib, Ceritinib, Cabozantinib, DWF, Afatinib, Ibrutinib, B43, KU004, Foretinib, KRCA-0008, PF-06439015, PF-06463922, Canertinib, GSA-10, GW2974, GW583340, WZ4002, CP-380736, D2667, Mubritinib, PD153035, PD168393, Pelitinib, PF-06459988, PF-06672131, PF-6422899, PKI-166, Reveromycin A, Tyrphostin 1, Tyrphostin 23, Tyrphostin 51, Tyrphostin AG 528, Tyrphostin AG 658, Tyrphostin AG 825, Tyrphostin AG 835, Tyrphostin AG 1478, Tyrphostin RG 13022, Tyrphostin RG 14620, B178, GSK1838705A, PD-161570, PD 173074, SU-5402, Roslin 2, Picropodophyllotoxin, PQ401, I-OMe-Tyrphostin AG 538, GNF 5837, GW441756, Tyrphostin AG 879, DMPQ, JNJ-10198409, PLX647, Trapidil, Tyrphostin A9, Tyrphostin AG 370, Lestaurtinib, DMH4, Geldanamycin, Genistein, GW2580, Herbimycin A, Lavendustin C, Midostaurin, NVP-BHG712, PD158780, PD-166866, PF-06273340, PP2, RPI, SU 11274, SU5614, Symadex, Tyrphostin AG 34, Tyrphostin AG 974, Tyrphostin AG 1007, UNC2881, Honokiol, SU1498, SKLB1002, CP-547632, JK-P3, KRN633, SC-1, ST638, SU 5416, Sulochrin, Tyrphostin SU 1498, 58567, rociletinib, Dacomitinib, Tivantinib, Neratinib, Ramucirumab, Masitinib, Vatalanib, Icotinib, XL-184, OSI-930, AB1010, Quizartinib, AZD9291, Tandutinib, HM61713, Brigantinib, Vemurafenib (PLX-4032), Semaxanib, AZD2171, Crenolanib, Damnacanthal, Fostamatinib, Motesanib, Radotinib, OSI-027, Linsitinib, BIX02189, PF-431396, PND-1186, PF-03814735, PF-431396, sirolimus, temsirolimus, everolimus, deforolimus, zotarolimus, BEZ235, INK128, Omipalisib, AZD8055, MHY1485, PI-103, KU-0063794, ETP-46464, GDC-0349, XL388, WYE-354, WYE-132, GSK1059615, WAY-600, PF-04691502, WYE-687, PP121, BGT226, AZD2014, PP242, CH5132799, P529, GDC-0980, GDC-0994, XMD8-92, Ulixertinib, FR180204, SCH772984, Trametinib, PD184352, PD98059, Selumetinib, PD325901, U0126, Pimasertinib, TAK-733, AZD8330, Binimetinib, PD318088, SL-327, Refametinib, GDC-0623, Cobimetinib, BI-847325, Adaphostin, GNF 2, PPY A, AIM-100, ASP 3026, LFM A13, PF 06465469, (−)-Terreic acid, AG-490, BIBU 1361, BIBX 1382, BMS 599626, CGP 52411, GW 583340, HDS 029, HKI 357, JNJ 28871063, WHI-P 154, PF 431396, PF 573228, FIN 1, PD 166285, SUN 11602, SR 140333, TCS 359, BMS 536924, NVP ADW 742, PQ 401, BMS 509744, CP 690550, NSC 33994, WHI-P 154, KB SRC 4, DDR1-IN-1, PF 04217903, PHA 665752, SU 16f, A 419259, AZM 475271, PP 1, PP 2, 1-Naphthyl PP1, Src I1, ANA 12, PD 90780, Ki 8751, Ki 20227, ZM 306416, ZM 323881, AEE 788, GTP 14564, PD 180970, R 1530, SU 6668, Toceranib, CEP-32496 (1-(3-((6,7-dimethoxyquinazolin-4-yl)oxy)phenyl)-3-(5-(1,1,1-trifluoro-2-methylpropan-2-yl)isoxazol-3-yl)urea), AZ 628 (4-(2-cyanopropan-2-yl)-N-(4-methyl-3-((3-methyl-4-oxo-3,4-dihydroquinazo-lin-6-yl)amino)phenyl) benzamide), Vemurafenib (PLX-4032), PLX-4720 (N-(3-(5-chloro-1H-pyrrolo[2,3-b]pyridine-3-carbonyl)-2,4-difluorophenyl)-propane-1-sulfonamide), SB 590885 ((E)-5-(2-(4-(2-(dimethylamino)ethoxy)phenyl)-4-(pyridin-4-yl)-1H-imidazo-1-5-yl)-2,3-dihydro-1H-inden-1-one oxime), and GDC-0879 ((E)-5-(2-(2-hydroxyethyl)-4-(pyridin-4-yl)-1H-imidazol-5-yl)-2,3-dihydro-1H-inden-1-one oxime). In certain implementations, PB is derived from crizotinib, certinib, alectinib, brigatinib, or JQ1.

The functioning of the PROTAC is typically dependent on the binding of the PB moiety to the protein of interest to bring the ULB moiety proximal to the POI following cleavage of the targeting group from the ULB. In certain embodiments, the PB may have an affinity for its target protein (K_{d}) of less than 1 mM (e.g. from 500 μM to 1 mM, etc.) or less than 500 μM (e.g., less than 450 μM, less than 400 μM, less than 350 μM, less than 300 μM, less than 250 μM, less than 200 μM, less than 150 μM, less than 100 μM, less than 100 μM, less than 50 μM, less than 10 μM, less than 1 μM, less than 500 nM, less than 100 nM, less than 50 nM, less than 10 nM, less than 1 nM, etc.).

The compounds, methods and compositions described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether, etc.) or alcohol (e.g., ethanol, etc.) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the disclosure may exist as tautomers or mixtures of tautomers, racemates, mixtures of racemates, stereoisomers, mixtures of stereoisomers, or combinations thereof. All tautomers are included within the scope of the compounds presented herein.

In certain embodiments, compounds described herein may be converted into an active PROTAC in vivo following endocytosis (and, particularly, targeting group assited endocytosis), and particularly, preferential endocytosis for disease causing cells such as proliferative cells (e.g., cancer cells). In certain embodiments, upon in vivo administration, the targeting group caged PROTAC is chemically converted to the biologically, pharmaceutically or therapeutically active form of the compound. Typically, the compounds of the present disclosure may be converted into compound capable of binding to the protein of interest, where cleavage of the targeting group induces ubiquitin recruitment within the cell following interaction with endogenous hydrolases. In other embodiments, the compounds of the present disclosure may be enzymatically metabolized by one or more steps or processes to the biologically, pharmaceutically or therapeutically active form of the compound during and/or following endocytosis.

The linker moiety may be chosen such that the PB moiety is able to bind to the POI and the decaged ULB group is able to degrade the POI following decaging. In some embodiments, L₁ or L₂ is a divalent hydrocarbon selected from saturated or unsaturated alkylene (e.g., branched alkylelene, linear alkylene, cycloalkylene, C₁-C₂₂ branched alkylelene, C₁-C₂₂ linear alkylene, C₃-C₂₂ cycloalkylene, C₁-C₁₀ branched alkylelene, C₁-C₁₀ linear alkylene, C₃-C₁₀ cycloalkylene, C₁-C₈ branched alkylelene, C₁-C₈ linear alkylene, C₃-C₈ cycloalkylene, etc.), C₁-C₂₂ saturated or unsaturated heteroalkylene (e.g., branched heteroalkylelene, linear heteroalkylene, heterocycloalkylene, C₁-C₂₂ branched heteroalkylelene, C₁-C₂₂ linear heteroalkylene, C₁-C₂₂ heterocycloalkylene, C₁-C₁₀ branched heteroalkylelene, C₁-C₁₀ linear heteroalkylene, C₁-C₁₀ heterocycloalkylene, C₁-C₈ branched heteroalkylelene, C₁-C₈ linear heteroalkylene, C₁-C₈ heterocycloalkylene, etc.), arylene (e.g., C₅-C₂₂ arylene, etc.), heteroarylene (e.g., C₁-C₂₂ heteroarylene, etc.), or combinations thereof. For example, L may comprise one or more of —(C(R^{a})(R^{a}))₁₋₈—, —(OC(R^{a})(R^{a}))₁₋₈—, —(OC(R^{a})(R^{a})—C(R^{a})(R^{a}))₁₋₈—, —N(R^{a})—, —O—, heteroarylene (e.g., five membered heteroarylene such as divalent triazole including

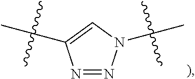 [SMILES: *c1cn(*)nn1]

### —S—S—, or —C(O)—;

wherein R^{a} is independently selected at each occurrence from hydrogen or alkyl (e.g., C₁-C₇ alkyl, C₁-C₄ alkyl, etc.).

In some embodiments, L is —(CH₂)₀₋₈—C(O)NH—(CH₂)₀₋₈—, —C(O)NH—(CH₂)₀₋₈—, —NHC(O)—(CH₂)₀₋₈—, —NH—(CH₂)₀₋₈—, or —C(O)—(CH₂)₀₋₈—, or combinations thereof. In some embodiments, L₂ has the structure —X₁—X₂—X₃—X₄—X₅— wherein X₁-X₅ are independently selected from absent, —C(O)—, —O—, —OC(O)—, —NR^{a}—, —N(R^{a})C(O)—, —(C(R^{a})(R^{a}))₁₋₈—, —(C(R^{a})(R^{a})C(R^{a})(R^{a})O)₁₋₈—, —S—S—, arylene, and heteroarylene; and

R^{a} is independently selected at each occurrence from hydrogen and alkyl. In certain implementations at least one of X₁-X₅ is heteroarylene (e.g., five membered heteroarylene such as divalent triazole including

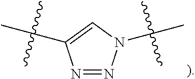 [SMILES: *c1cn(*)nn1]

In some embodiments, L₂ has the structure —(C(Rₐ)(Rₐ))ₘ—X₃—(C(Rₐ)(Rₐ))ₙ—, wherein m and n are independently selected from integers from 0-8 (e.g., 0, 1, 2, 3, 4, 5, 6, 7, 8). In particular embodiments, X₃ is heteroarylene (e.g., five membered heteroarylene such as divalent triazole including

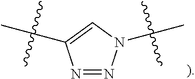 [SMILES: *c1cn(*)nn1]

The disclosure includes a pharmaceutical composition comprising at least one compound described herein (e.g., Compounds having the structure of (I), (III), (IV), (V), (VI), (VII), Compound 1, Compound 2, Compound 3, etc.) and at least one pharmaceutically acceptable carrier, diluent, or diluent. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

The pharmaceutical composition may comprise a compound disclosed herein (e.g., Compounds having the structure of (I), (III), (IV), (V), (VI), (VII), Compound 1, Compound 2, Compound 3, Compound 4, etc.) and one or more pharmaceutically acceptable salts, carriers, or diluents. In specific embodiments, the compound is formulated as a topical composition (e.g., ointment, gel, etc.). In some embodiments, the composition comprises from 0.1%-90% (e.g., 0.1%-50%, 0.1%-20%, 0.1%-10%, etc.) of the compound by weight of the composition.

The disclosure includes a method of treating or preventing a disease which may be associated with and/or caused by overexpression and/or uncontrolled activation of certain proteins (e.g., tyrosine kinase) in a subject in need thereof. The disclosure further includes a method of treating or preventing a cancer associated with and/or caused by a specific protein of interest (e.g., an oncogenic tyrosine kinase, etc.) in a subject in need thereof. In certain embodiments, the disease comprises a cancer. In some implementations, the tyrosine kinase is c-ABL and/or BCR-ABL. In yet other embodiments, the cancer is chronic myelogenous leukemia (CML).

Examples of cancers that can be treated or prevented by the present disclosure include but are not limited to: squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma, myeloproliferative diseases, large B cell lymphoma, and B cell Lymphoma. In certain embodiments, the proliferative disease is melanoma, leukemia, lymphoma, or retinal blastoma.

The methods of the disclosure may comprise administering to the subject a therapeutically effective amount of at least one compound of the disclosure (e.g., Compounds having the structure of (I), (III), (IV), (V), (VI), (VII), Compound 1, Compound 2, Compound 3, Compound 4, etc.), which is optionally formulated in a pharmaceutical composition. In certain embodiments, the method further comprises administering to the subject an additional therapeutic agent that treats or prevents cancer. The compound may be a compound for the treatment of a proliferative disease. In certain embodiments, the compound may be a compound for the manufacture of a medicament for the treatment of a proliferative disease. The method for the treatment of a proliferative disease may comprise the administration of a compound or pharmaceutical composition as disclosed herein.

In certain embodiments, administering the compound of the disclosure to the subject allows for administering a lower dose of the additional therapeutic agent as compared to the dose of the additional therapeutic agent alone that is required to achieve similar results in treating or preventing a cancer in the subject. For example, in certain embodiments, the compounds disclosed herein may enhance the anti-cancer activity of the additional therapeutic compound, thereby allowing for a lower dose of the additional therapeutic compound to provide the same effect. In certain embodiments, the compounds and the therapeutic agent are co-administered to the subject. In other embodiments, the compound of the disclosure and the therapeutic agent are coformulated and co-administered to the subject.

In certain embodiments, the subject is a mammal. In other embodiments, the mammal is a human.

The therapeutically effective amount or dose of a compound of the present disclosure depends on the age, sex and weight of the patient, the current medical condition of the patient and the progression of a cancer in the patient being treated. The skilled artisan is able to determine appropriate dosages depending on these and other factors. For example, the therapeutically effective amount may be determined based on the amount of decaged PROTAC required to treat a proliferative disease of interest. For example, the therapeutically effective amount may be based on more than 20% or more than 30% or more than 40% or more than 50% or more than 60% or more than 70% or more than 80% or more than 90% or more than 95% or more than 95% or more than 99% or 100% being decaged following endocytosis f the compounds described herein by weight of the composition.

For example, a suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the disclosure is optionally given continuously; alternatively, the dose of drug being administered is temporarily reduced or temporarily suspended for a certain length of time. The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds for use in the method of the disclosure (e.g., Compounds having the structure of (I), (III), (IV), (V), (VI), (VII), Compound 1, Compound 2, Compound 3, Compound 4, etc.) may be formulated in unit dosage form. Typically, unit dosage forms are physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Many applications of the targeting group conjugated PROTACs of the present disclosure may involve cell surface localization or targeting. However, the general methods of this invention may also be extended to include internalization of the compound in cells. In these embodiments, constructs that incorporate an internalization or targeting moiety that causes the cell to internalize a PROTAC that only becomes active following the internatlization. In this regard, the disclosure concerns an approach for internalizing PROTACs, into target cells. In this way, targeted internalization occurs instead of the non-specific internalization that would occur by use of the internalization agent without the localization agent.

These targeting group conjugated PROTACs may have enhanced cellular internalization of the PROTAC. Internalization of the targeting group conjugated PROTAC can be accomplished in various ways. For example, the internalization moiety (also referred to herein as a targeting group) may bind to a recycling receptor, such as a folate receptor. For binding to a folate receptor, the internalization moiety can, for example, include folate or methotrexate, or a folate analog binding to a folate receptor. In other embodiments, the internalization moiety includes a peptide that enhances non-receptor mediated internalization, such as the HIV-1 tat protein, transportan, MAP (model amphipathic peptide), antennapedia peptide, also known as penetratin, and proteins or peptides that are known to internalize moieties to which they are attached. Nagahara et al., Nature Medicine, 4(12):1449 1998 discloses the use of tat in fusion proteins, which is hereby incorporated by reference in its entirety. Other cell penetrating peptides are are disclosed in Thoren et al., FEBS Letters 482: 265-268 (2000); Mazel et al., Anti-Cancer Drugs 12: 107-116 (2001); Hallbrink et al., Biochim. et Biophys. Acta 15, each of which are hereby incorporated by reference in their entirety.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Some L₂ linkers of the present disclosure, particularly those with disulfide linkages, operate via intracellular compound mediated reduction or hydrolysis of the linker between the ULB and the TG moiety. In some embodiments, compounds of the present disclosure (e.g., Compounds having the structure of (I), (III), (IV), (V), (VI), (VII), Compound 1, Compound 2, Compound 3, Compound 4, etc.) may be administered in combination with a compound which undergoes endocytosis and provides the intracellular medium with an increased concentration of the hydrolysis or reductive compound. Such combination administration may increase the efficacy of the compounds of the present disclosure (e.g., as compared to IC₅₀ values to administration alone). For example, in compounds having a disulfide linkage, intracellular gluthianone (GSH) may be responsible for reduction and subsequent spontaneous intramolecular cyclization to active the compound. Gluthianone or a compound which undergoes intracellular biosynthesis to form gluthianone (e.g., S—Ac-GSH) may be administered in combination with a compound of the present disclosure. Such combination treatment may increase intracellular gluthianone levels resulting in more efficient intracellular decaging. This combination administration may occur sequentially or simultaneously to administration of the compound of the present disclosure. For example, the compound which undergoes endocytosis and provides the intracellular medium with an increased concentration of the hydrolysis or reductive compound may be administered for a time period before administration of the compounds of the present disclosure (e.g., 12 hours prior) to build up an increased intracellular concentration of the hydrolysis or reductive compound.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application. Unless otherwise apparent from context, the sum of all weight percentages should not exceed 100% and all percentages in relation to components refer to percentages by weight of the composition.

### EXAMPLES

The following examples illustrate specific aspects of the instant description. The examples should not be construed as limiting, as the example merely provides specific understanding and practice of the embodiments and its various aspects.

### Example 1

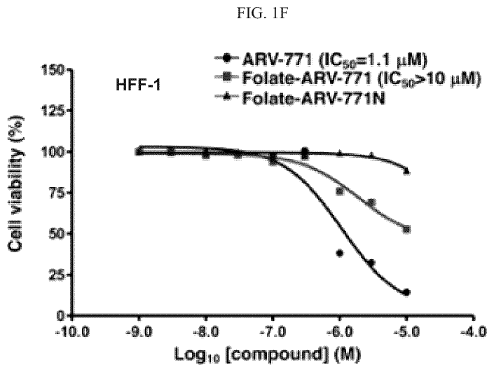

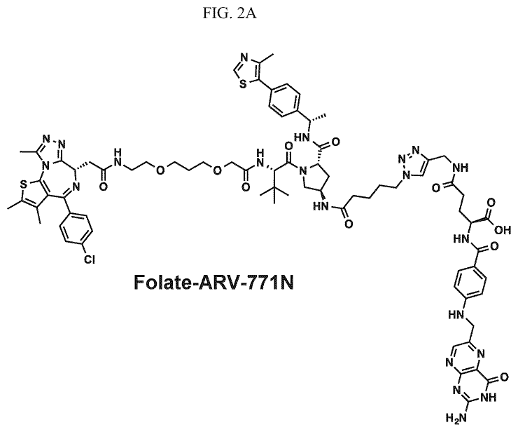

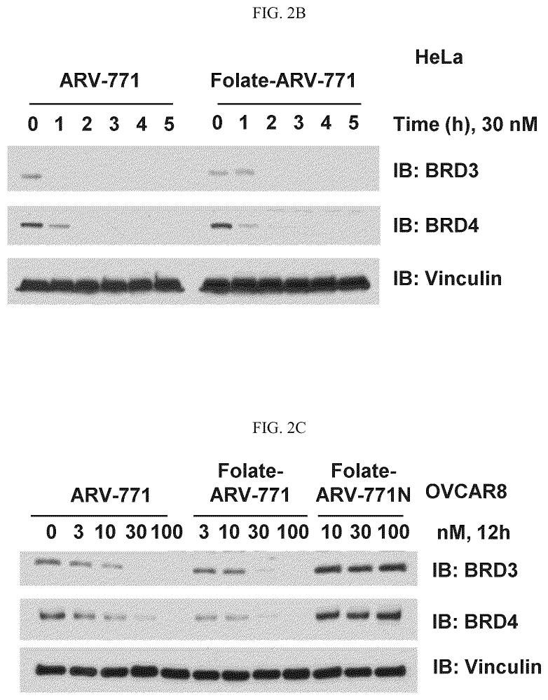

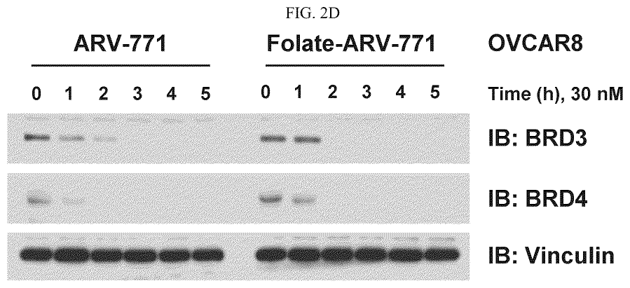

A negative control of folate-PROTAC, Folate-ARV-771N, was designed by replacing the ester bond to a non-cleavable amide bond (FIG. 2A), thus Folate-ARV-771N is resistant to cleavage and remains inactive even after entering into cells.

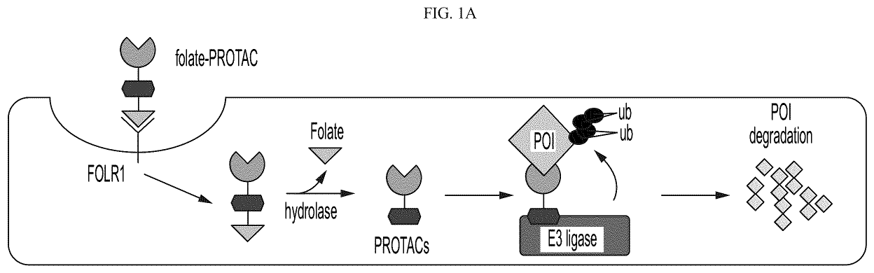

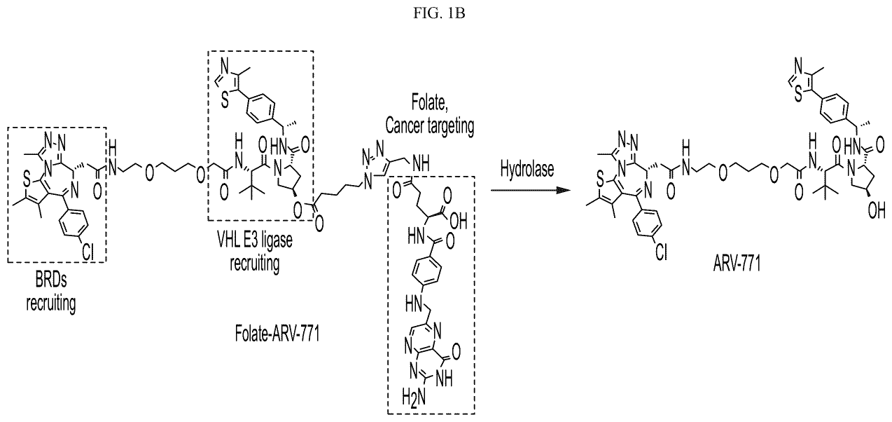

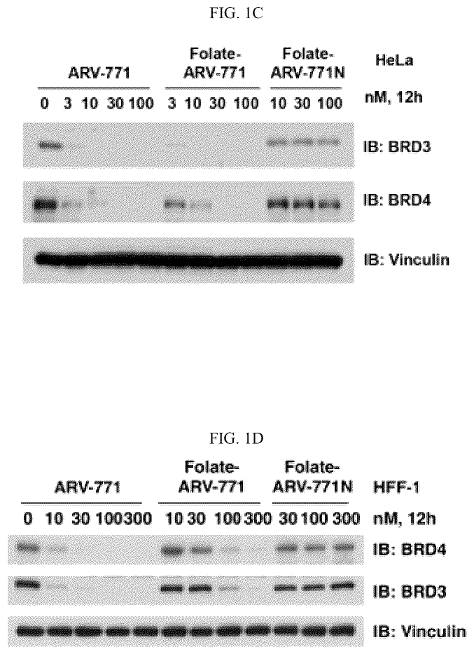

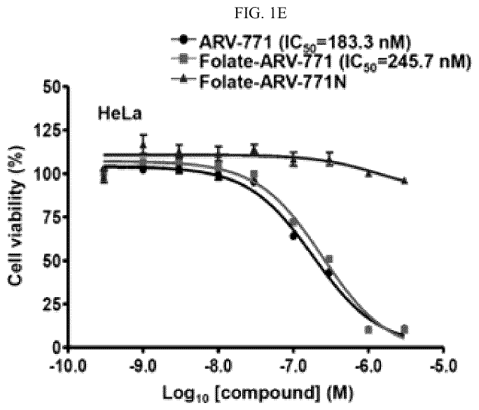

Folate-ARV-771 and its negative control, Folate-ARV-771N, were synthesized through a Cu-Catalyzed Azide-Alkyne Cycloaddition (CuAAC) reaction to conjugate alkyne-modified folic acid and azide-modified ARV-771 ester (Schemes 51 and S2). As HeLa cells have relatively high level of FOLR1 expression, the effect of Folate-ARV-771 in HeLa cells as determined. Human fibroblast cells (HFF-1) have a lower level of FOLR1 expression. Notably, in HeLA cancer cells, ARV-771, Folate-ARV-771 degraded BRD4 efficiently with DC₅₀<10 nM, while the non-cleavable negative control, Folate-ARV-771N, was incapable of degrading BRDs even at 100 nM (FIGS. 1C and 2B). In contrast, in HFF-1 normal cells, the DC₅₀ of Folate-ARV-771 was ˜10 fold that of ARV-771 (FIG. 1D, 100 nM as compared to 10 nM in cancer cells). Furthermore, Folate-ARV-771 had a comparable IC₅₀ (245.7 nM) with ARV-771 (183.3 nM) for HeLa cells (FIG. 1E). In HFF-1 normal cells, FOLATE-ARV-771 was much less efficient than ARV-771, with an IC₅₀ of >10 μM versus 1.1 μM in the cancer cells (FIG. 1F). These data illustrate targeting group caged PROTACs are specifically enrichied the cancer cell as compared to the normal cell. Furthermore, that enrichment leads to cancer cell specific degradation of the protein of interest in the cancer cell as compared to a healthy control cell.

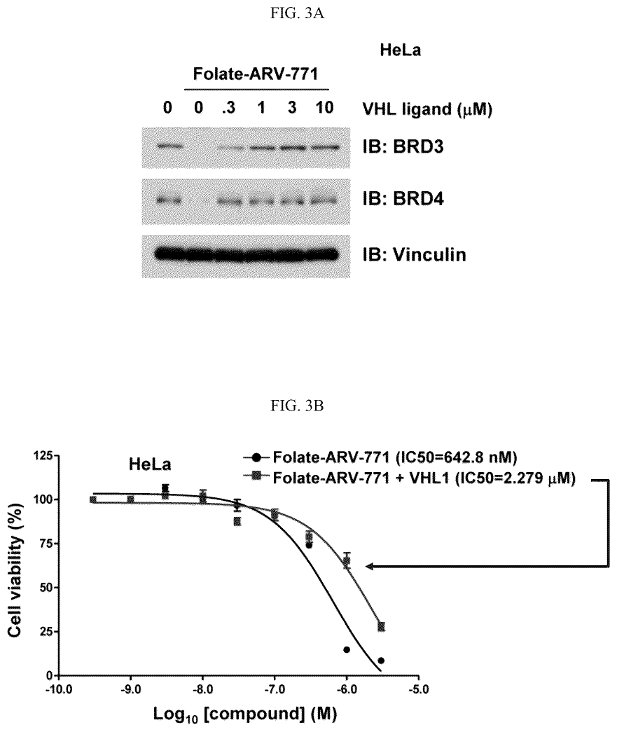

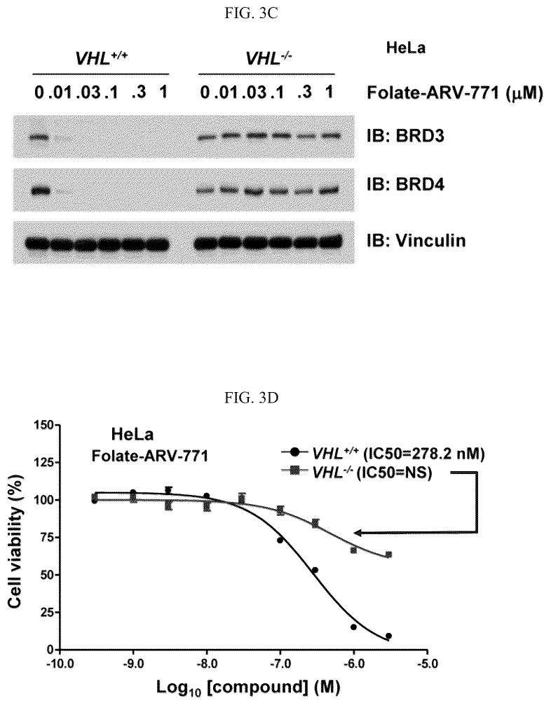

Given that ovary cancer has relatively high FOLR1 expression, the effect of Folate-ARV-771 in an ovary cancer cell line, OVCAR-8, was measured (FIGS. 2C and 2D). Notably, Folate-ARV-771, but not Folate-ARV-771N, degraded BRDs as efficiently efficiently in OVCAR-8 cells (FIGS. 2C and 2D). To show that the degradation of the POI by folate caged PROTACs depends on VHL E3 ligase, HeLa cells were also treated with free VHL ligand (Ac-VHL) together with Folate-ARV-771. It was found that co-treatment of Ac-VHL effectively blocked the degradation of BRDs by Folate-ARV-771 (FIG. 3A) and increased the IC₅₀ of Folate-ARV-771 (FIG. 3B). Moreover, deletion of endogenous VHL1 completely abolished the effect of Folate-ARV-771 on both degradation of BRDs and IC₅₀ (FIGS. 3C and 3D), further supporting the dependence of Folate-ARV-771 on endogenous VHL E3 ubiquitin ligase. These results demonstrate that folate conjugation will not introduce non-specific function to original PROTACs.

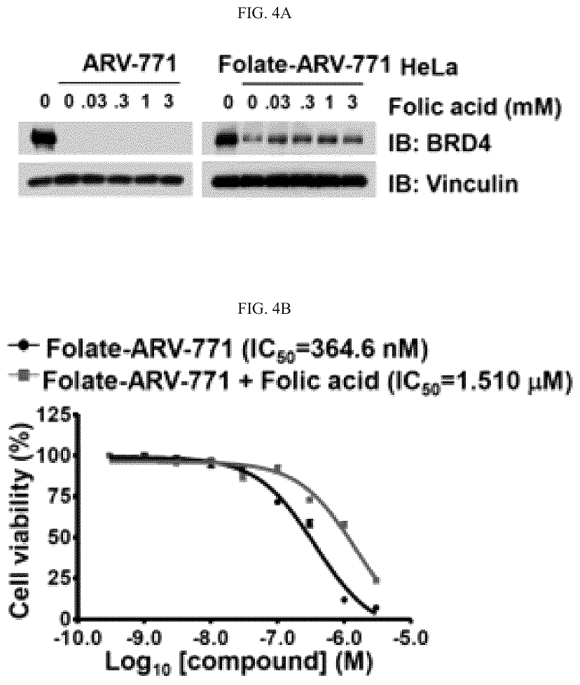

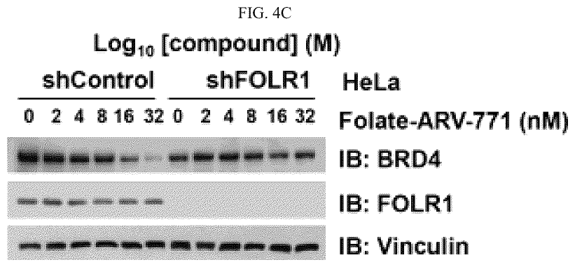

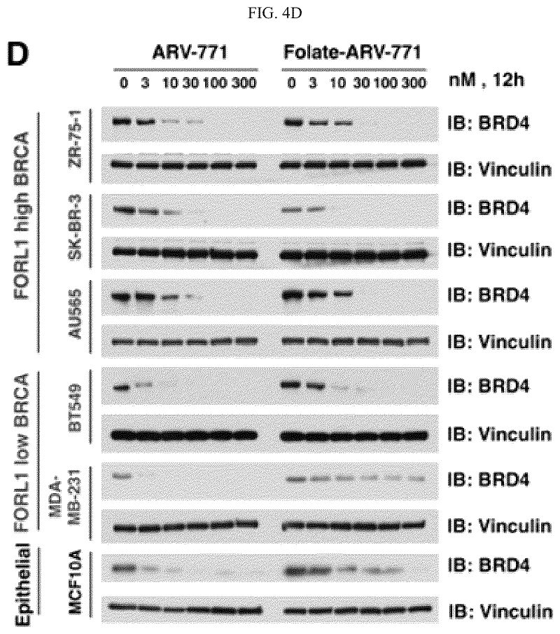

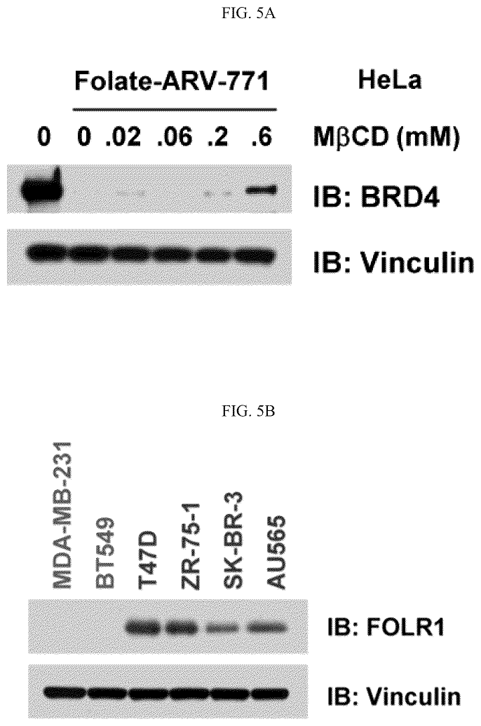

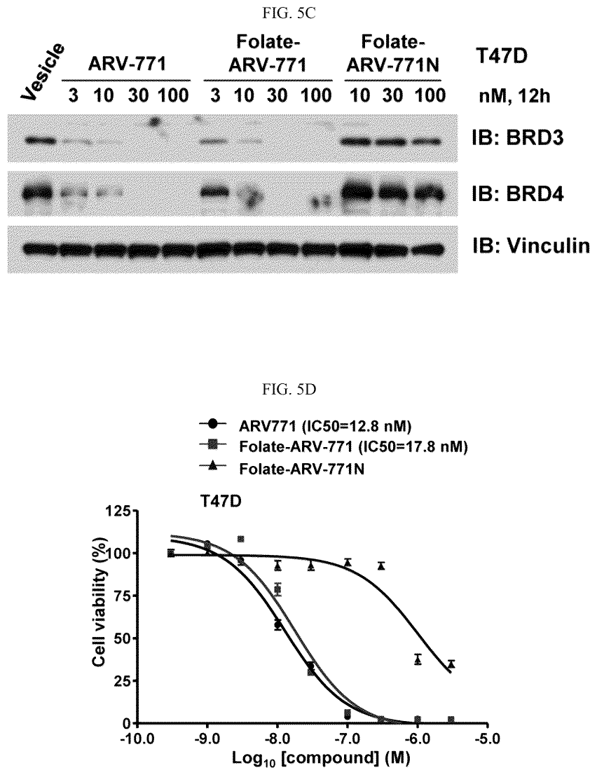

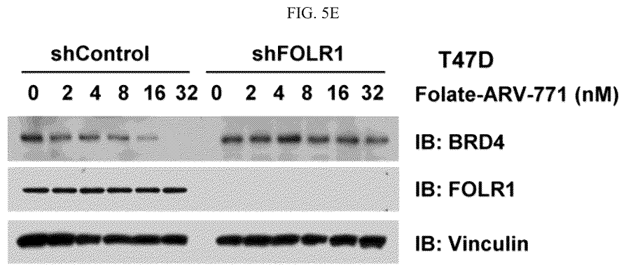

The entry of folate-conjugates into cells largely depends on its receptor FOLR1 on cancer cell membrane, and FOLR1-mediated drug entry can be antagonized by free folic acid. To this end, HeLa cells were pretreated with free folic acid and then challenged with either ARV-771 or Folate-ARV-771. It was found that free folic acid antagonized the ability of Folate-ARV-771 in degrading BRD4, but not ARV-771 (FIG. 4A). As such, pretreatment with free folic acid significantly increased the IC₅₀ of Folate-ARV-771 from 364.4 nM to 1.51 μM (FIG. 4B). Furthermore, depletion of endogenous FOLR1 also eliminated the effect of Folate-ARV-771 in degrading BRD4 in HeLa cells (FIG. 4C). After binding with FOLR1, folate-conjugates take advantage of the endocytosis process to enter cells. To further examine the role of endocytosis in this process, HeLa cells were pretreated with an endocytosis inhibitor, MβCD, and found that MβCD efficiently blocked the effect of Folate-AVR-771 in degrading BRD4 (FIG. 5A).

To further determine the critical role of FOLR1 for dictating the activity of folate-PROTAC, the effects of Folate-ARV-771 were measured in FOLR1-negative breast epithelial cell MCF10A versus a panel of breast cancer (BRCA) cell lines with distinct FOLR1 expression levels (FIG. 5B). Notably, Folate-ARV-771 degraded BRD4 efficiently in those BRCA cells with relatively high FOLR1 expression, including T47D, ZR-75-1, SK-BR-3 and AU565, with the DC₅₀ much lower than that of ARV-771 (FIG. 4D, 5C-5E). In contrast, in either normal breast epithelial MCF10A cells or FOLR1-low cells (BT549 and MDA-MB-231), Folate-ARV-771 was much less efficient in degrading BRD4 compared with ARV-771. There was a 10-100 fold difference in DC₅₁ values for Folate-ARV-771 as compared to ARV-771 (FIG. 4D). Taken together, these results indicated that Folate-ARV-771 degrades BRDs in a FOLR1-dependent manner, particularly in cancer cells.

### Example 2

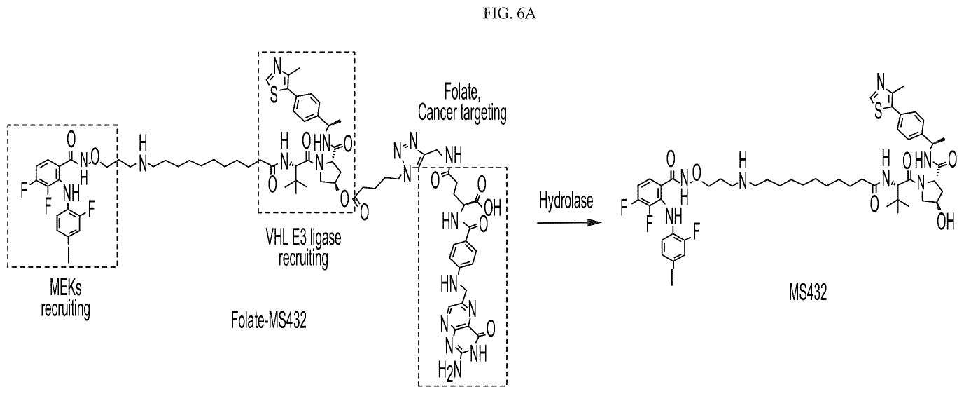

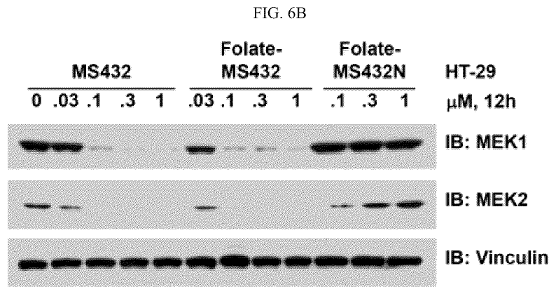

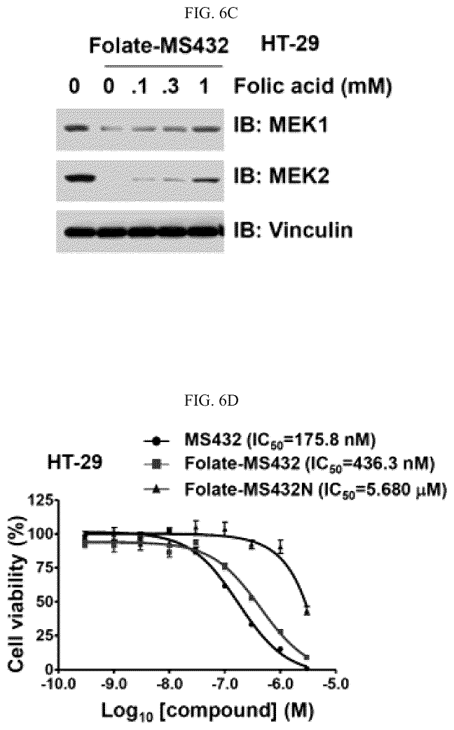

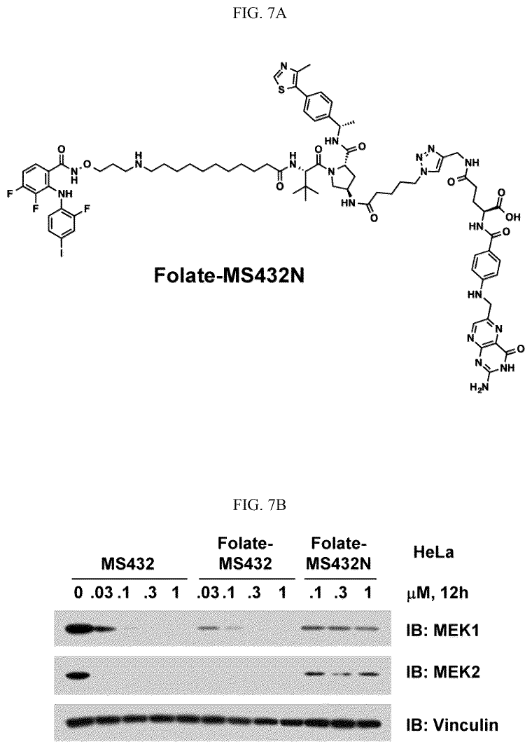

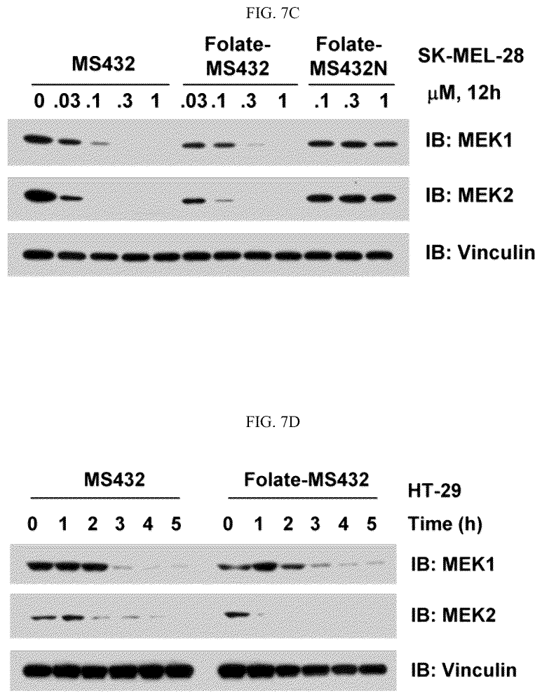

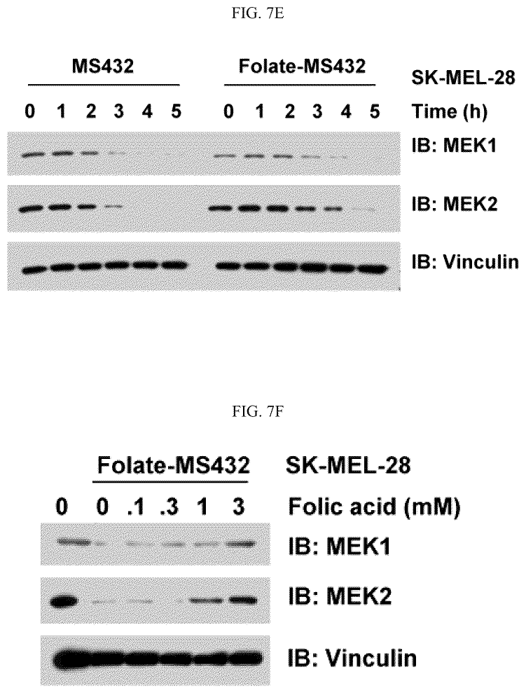

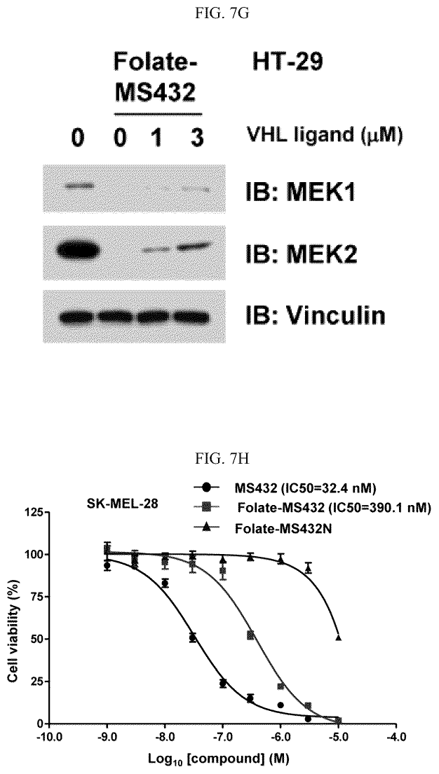

Since MEK/ERK signaling is critical for the survival of BRAF mutant cancer cells, the effect of Folate-MS432 in BRAF-V600E mutant harboring cells was also evaluated, including a colorectal cancer (CRC) cell line, HT29, and a melanoma cell line, SK-MEL-28. As expected, Folate-MS432 degraded MEK1 and MEK2 in both HT29 and SK-MEL-28 cells in dose- and time-dependent manners, while Folate-MS432N could not do so (FIG. 6B, FIGS. 7C-7E). Moreover, pretreatment of folic acid blocked the effect of Folate-MS432 in degrading MEK1 and MEK2 in both HT-29 (FIG. 6C) and SK-MEL-28 cells (FIG. 7F). Furthermore, pretreatment with the VHL ligand, Ac-VHL, also blocked the degradation of MEK1/2 in HT-29 cells (FIG. 7G). The cell viability of both HT-29 and SK-MEL-28 cells after treatment with either MS432, Folate-MS432 or Folate-MS432N was also measured. In HT-29 cells, the IC₅₀ of MS432 and Folate-MS432 was 175.8 nM and 436.3 nM, respectively (FIG. 6D). In SK-MEL-28 cells, the IC₅₀ of MS432 and Folate-MS432 was 32.4 nM and 390.1 nM, respectively (FIG. 7H). These results together indicated that Folate-MS432 degrades MEK1/2 largely in a FOLR1-dependent manner.

### Example 3

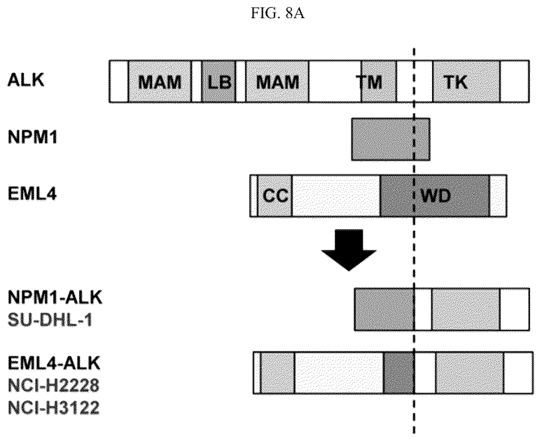

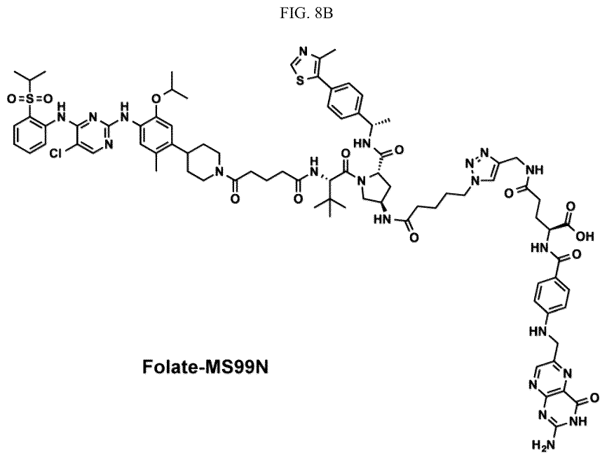

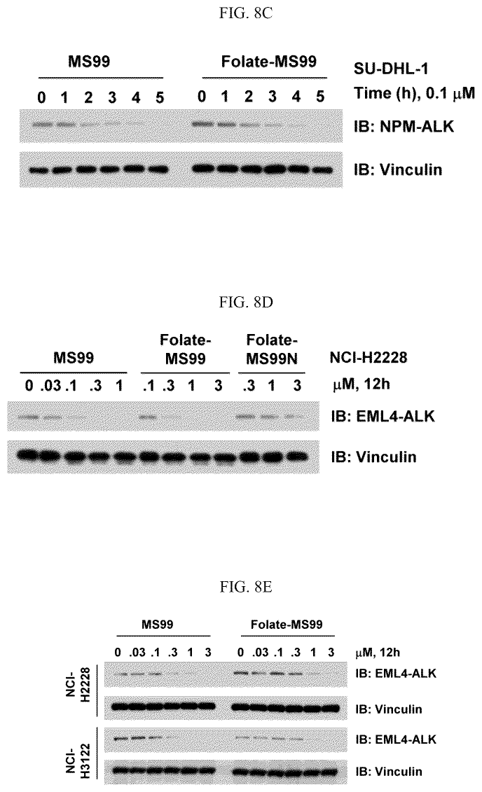

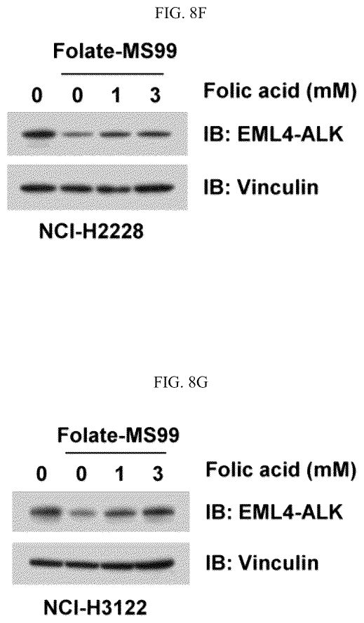

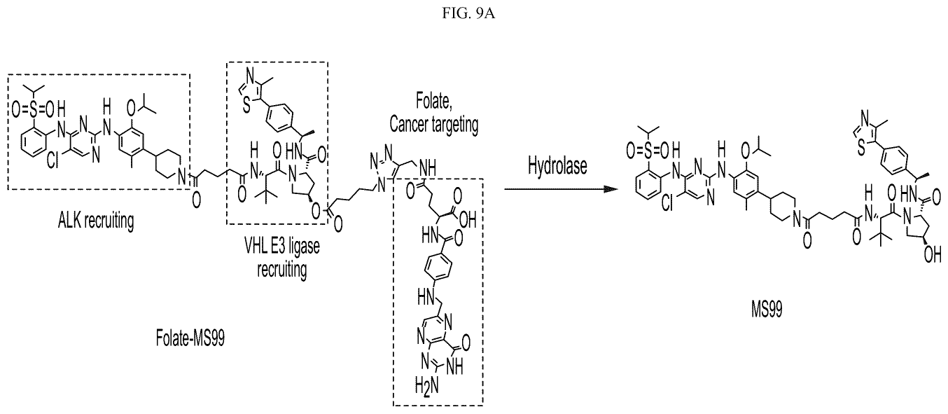

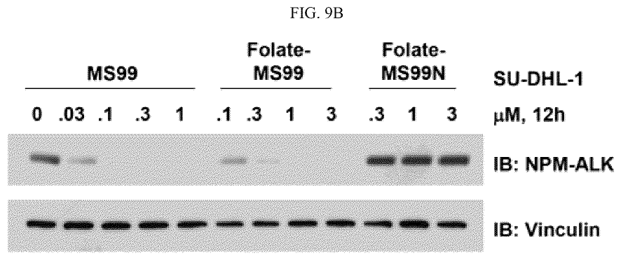

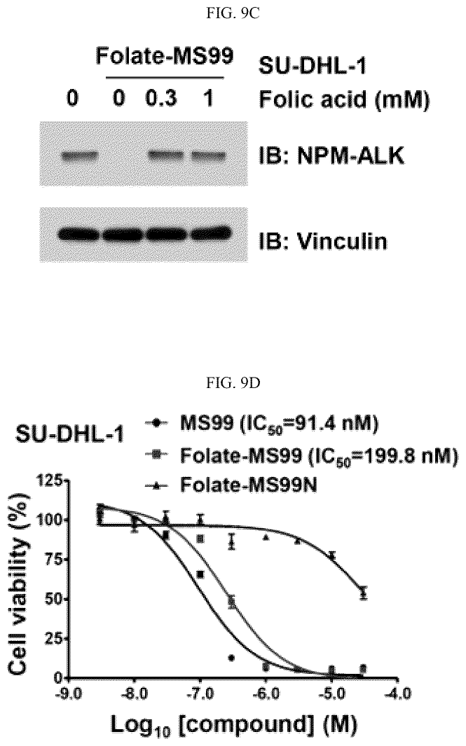

Anaplastic lymphoma kinase (ALK) fusion proteins are the drivers in several types of cancer, including the EML4-ALK fusion in non-small cell lung cancer (NSCLC) ¹⁷ and NPM-ALK fusion in leukemia ¹⁸ (FIG. 8A). These fusion proteins are constitutively active and confer resistance to ALK inhibitor in clinic, while ALK degraders are expected to overcome such drug resistance. To this end, a new folate-conjugated VHL-based degrader, Folate-MS99, and its negative control, Folate-MS99N, were synthesized (FIGS. 9A, 8B, Scheme S5-S7). The efficiency of Folate-MS99 in degrading ALK fusion proteins in SU-DHL-1 cells, an anaplastic large cell lymphoma (ALCL) cell line with NPM-ALK fusion was determined. Both MS99 and Folate-MS99 degraded NPM-ALK fusion protein efficiently, with a DC₅₀ of 30 nM for MS99 and 100 nM for Folate-MS99 (FIGS. 9B and 8C). On the other hand, Folate-MS99N was incapable of degrading NPM-ALK even at 3 μM (FIG. 9B). Furthermore, Folate-MS99 efficiently degraded the EML4-ALK fusion proteins in two NSCLC cell lines, NCI-H2228 and NCI-H3122 cells (FIGS. 8D and 8E). More importantly, pretreatment with free folic acid antagonized the effect of Folate-MS99 in degrading NPM-ALK fusion protein in SU-DHL-1 cells (FIG. 9C) and EML4-ALK fusion protein in both NSCLC cells (FIGS. 8F and 8G). The cell viability of SU-DHL-1 cells after the treatment with MS99, Folate-MS99, or Folate-MS99N was measured. The IC₅₀ of MS99 and Folate-MS99 were 91.4 nM and 199.8 nM, respectively (FIG. 9D). These results indicated that Folate-MS99 is efficient in degrading ALK fusion proteins in a FOLR1-dependent manner.

### Example 4

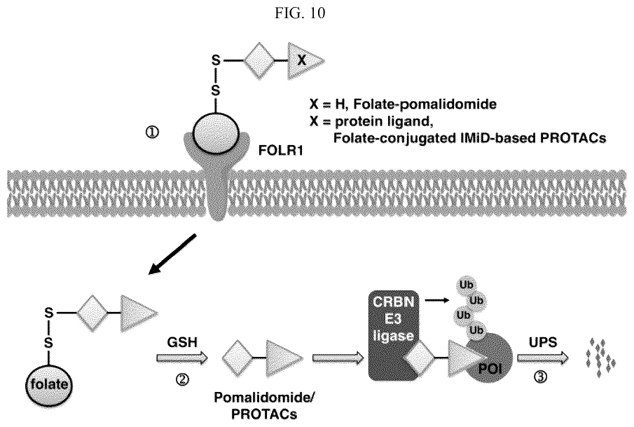

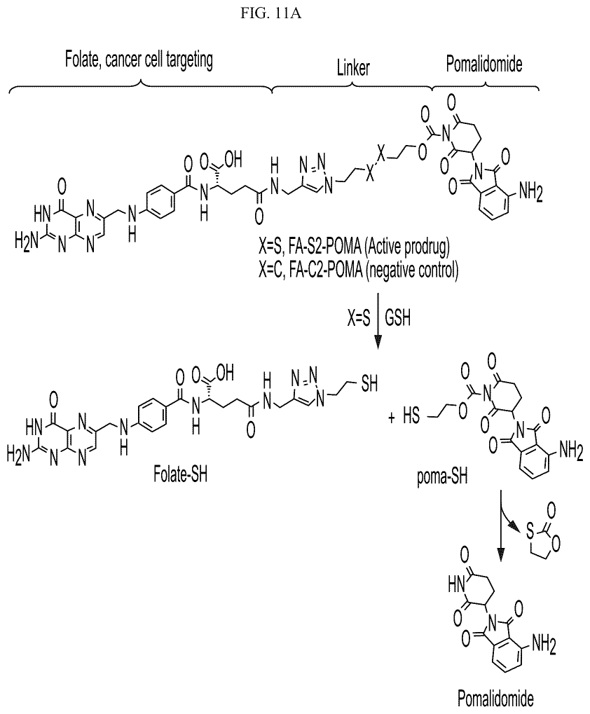

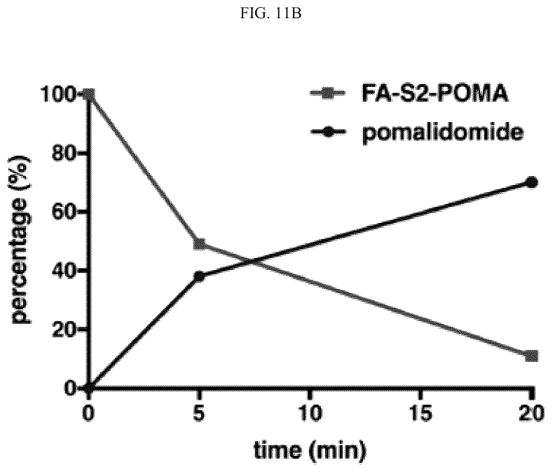

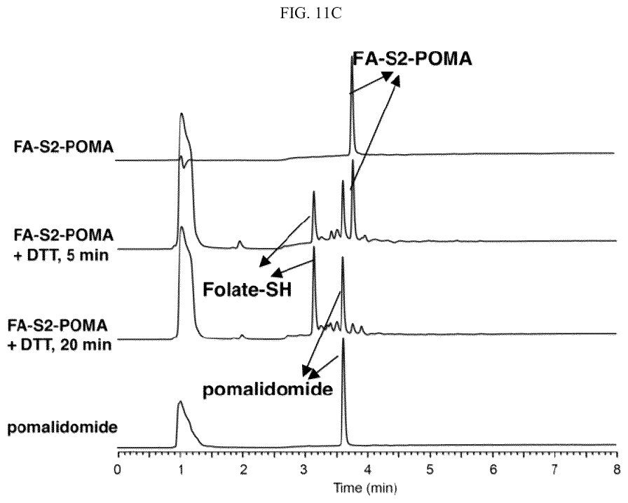

Pomalidomide was also assessed for IMiD ULB groups in the targeting group caging PROTAC strategy of the present disclosure. Specifically, a folate group was incorporated onto the glutarimide NH group of pomalidomide and the cancel cell specific drug delivery was assessed. A reduction cleavable linker was assesd such that activation of the caged PROTAC occurs via intracellular gluthianone (GSH) reduction and subsequent production of pomalidomide active for ubiquitin reqruitment and subsequent cellular degradation. A schematic of this process is illustrated in FIG. 10.

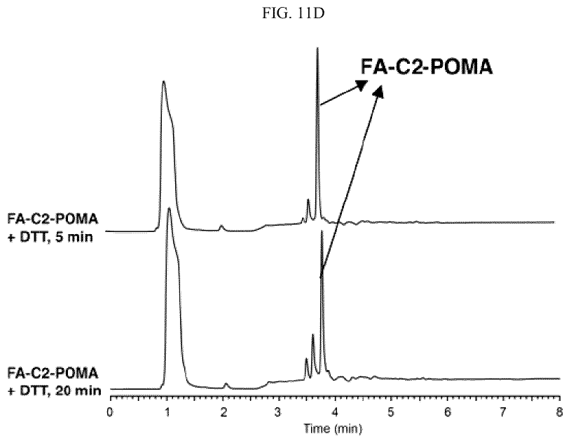

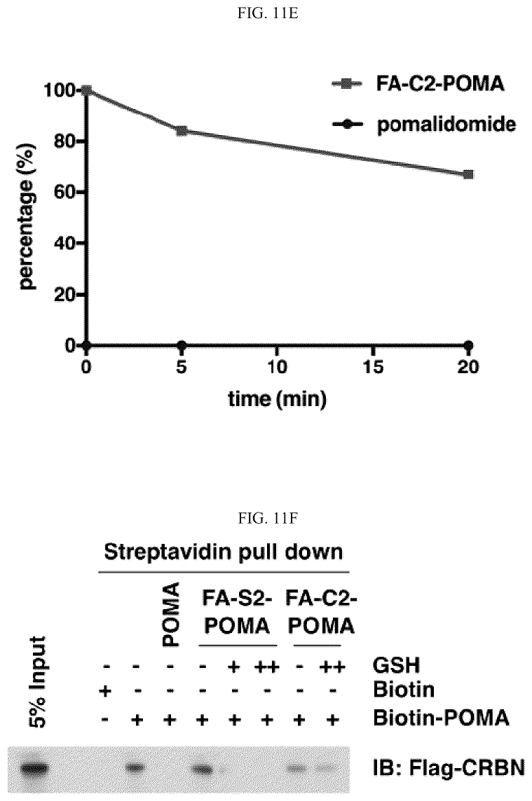

Conjugation of the targeting group ligand to the glutarimide NH group on pomalidomide was shown to render pomalidomide inert until folate mediated endocytosis occured. To release pomalidomide after its entry into cancer cells as guided by the folate group, pomalidomide and folic acid were conjugated via a reduction-cleavable disulfide bond (—S—S—), which can be cleaved by endogenous GSH in cells, followed by spontaneous intramolecular cyclization to release the active pomalidomide and 1,2-oxathiolan-2-one. This can be seen in FIG. 11A. A non-cleavable negative control of FA-S2-POMA was produced by replacing the disulfide linker with ethylene (—CH₂—CH₂—) which cannot be easily cleaved by GSH (FA-C2-POMA).

FA-S2-POMA and the negative control FA-C2-POMA were incubated with dithiothreitol (DTT) under physiological conditions (37° C. in phosphate buffered saline (PBS)) and then subjected to HPLC analysis. Notably, the addition of DTT led to the efficient release of pomalidomide from FA-S2-POMA, but not from FA-C2-POMA as shown in FIGS. 11B-E.

Additionally, FA-S2-POMA and FA-C2-POMA in the caged, inert form were shown to not compete with biotinylated-pomalidomide to bind the CRBNE3 ubiquitin ligase with Flag-tagged cereblon (Flag-CRBN). Streptavadin pull down assays were performed on cell lysis derived from HEK293T (kidney) cells. Cell lysis was was incubated with biotin or biotin-POMA, with or without pomalidomide, FA-S2_POMA or FA-C2-POMA for 1 hour. Additionally, some cell lysates had been pretreated with GSH. Pull down assays are shown in FIG. 11F. These results suggest that FA-S2-POMA as a folate-caged pomalidomide can release pomalidomide to form the active upon treatment with a reducing agent such as GSH under physiological conditions.

### Example 5

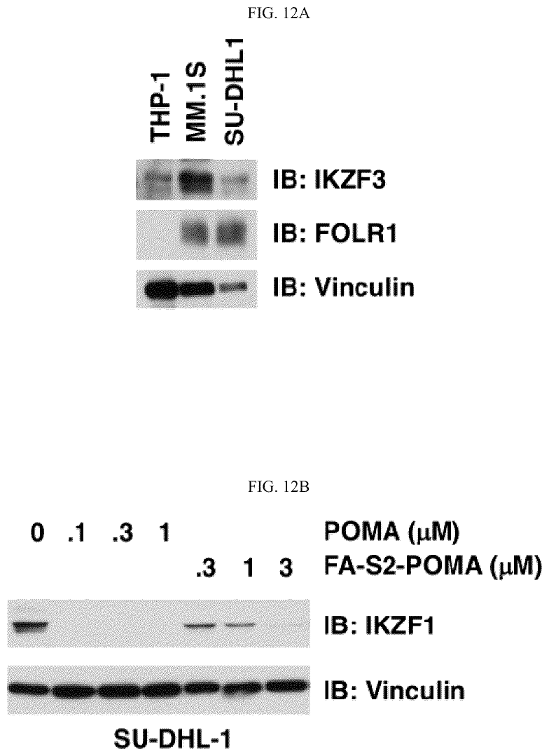

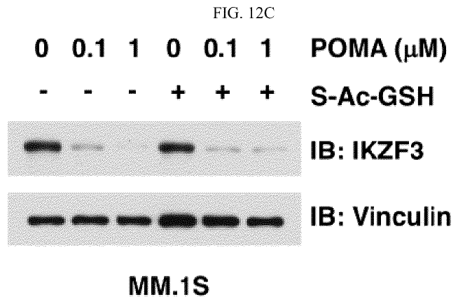

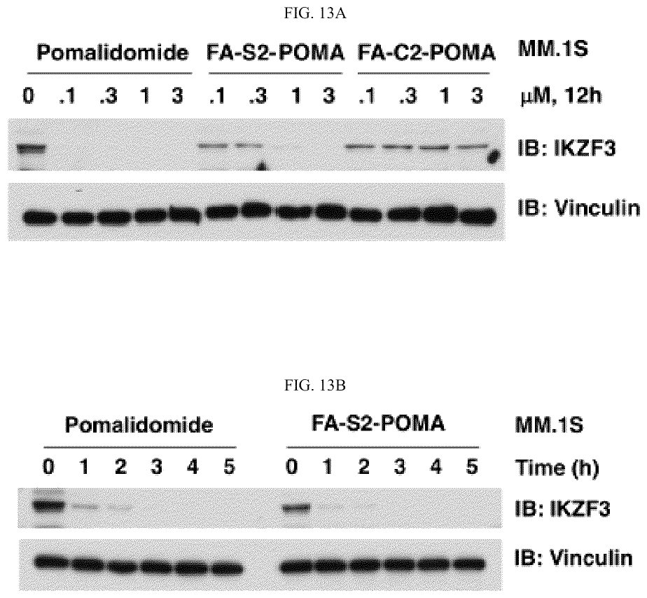

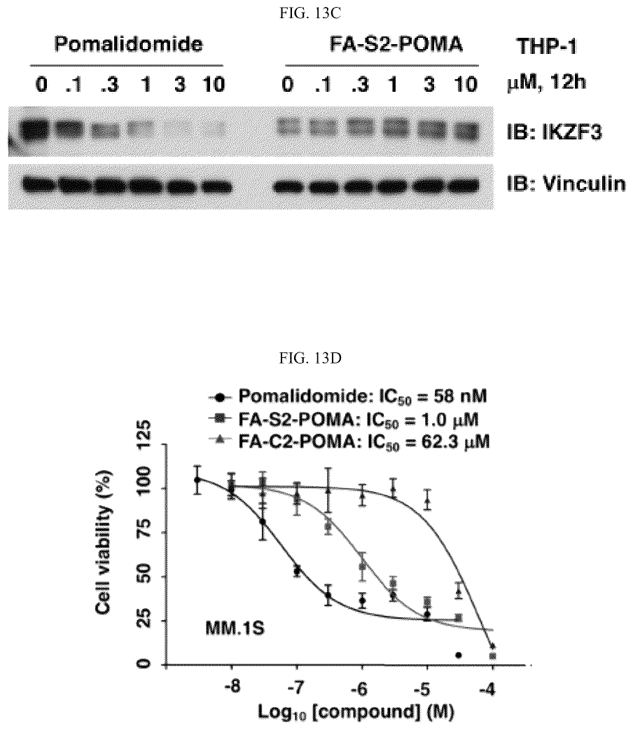

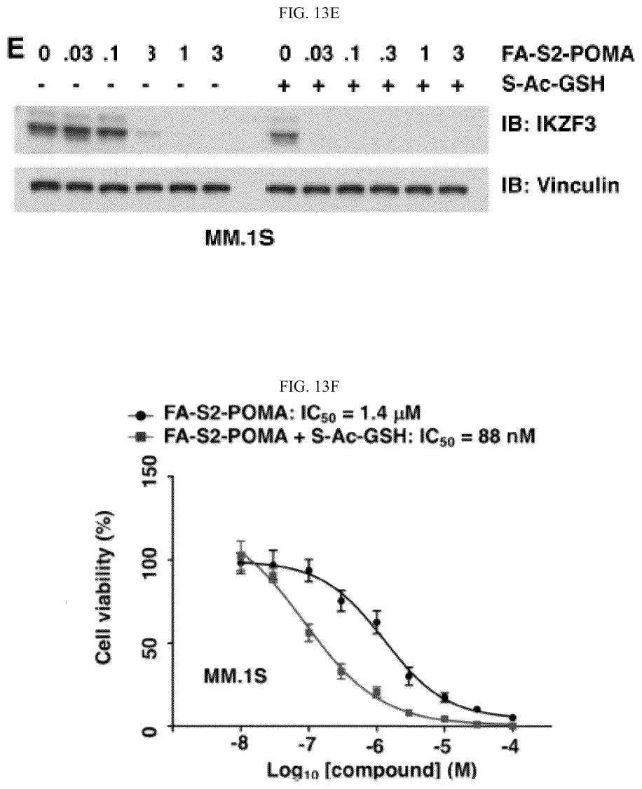

Folate-caged pomalidomide was shown to be effectively guided and delivered into cells that express FOLR1, but not cells without FOLR1 expression. Delivery into cells resulted in effective degradation of pomalidomide-CRBN neo substrates Ikaros family zinc finger (IKZF) proteins IKZF1 and IKZF3. Western blotting was performed on IKZF3 and FOLR1 in THP-1 (AML), MM.1S (immunoglobulin A lambda myeloma), and SU-DHL-1 (large cell lymphoma; diffuse histiocytic lymphoma) cells. Results are shown in FIGS. 12A-C. Notably, pomalidomide degrade IKZF3 or IKZF1 in all three cell lines in a concentration dependent manner as can be seen in FIGS. 13A-C(IKZF3 in MM.2S and THP-1 cells) and 12B (IZKF1 in SU-DHL-1 cells). On the other hand, FA-S2-POMA degraded IKZF3 or IKZF1 in FOLR1-positive MM.1S (FIG. 13A-B, IKZF3) and SU-DHL-1 cells (FIG. 12B, IKZF1), but not in FOLR1-negative THP-1 cells (FIG. 13C, IKZF3). FA-S2-POMA selectively targets FOLR1-expressing cells over FOLR1-negative cells. Moreover, the non-cleavable negative control FA-C2-POMA was ineffective in degrading IKZF3 in MM.1S cells (FIG. 13A). Without wishing to be bound by theory, this lack of activity was likely due to the linker inability to undergo the decaging process.

FA-S2-POMA effectively inhibited the proliferation of MM.1S cells with an IC₅₀ of 1.0 μM, which is more potent than FA-C2 POMA (IC₅₀=62.3 μM) but less potent than pomalidomide (IC₅₀=58 nM) (FIG. 13D), suggesting that the decaging process is likely pivotal for the effectiveness of FA-S2-POMA. The anti-proliferation effect of these compounds is consistent with their effect on degrading IKZF3 in MM. 1S cells (FIG. 13A).

Given that the disulfide bond between the folate group and the pomalidomide in FA-S2-POMA is presumably reduced mainly by GSH, we further treated MM.1S cells with S-Acetyl-L-Glutathione (S—Ac-GSH), which is stable under physiological conditions and can be directly taken up by cells and converted into GSH by intracellular thioesterases to increase the intracellular levels of GSH. Pretreatment with S—Ac-GSH significantly increased the potentcy of FA-S2-POMA (FIG. 13E), but not pomalidomide (FIG. 12C), in degrading IKZF3 in MM.1S cells. Consistent with these results, pretreatment with S—Ac-GSH significantly increased potency of FA-S2-POMA in inhibiting the proliferation of MM.1S cells (FIG. 13F) to a comparable level to pomalidomide. These date further support that folate-guided cellular uptake, FA-S2-POMA is cleaved by intracellular GSH, leading to the release of pomalidomide, which, in turn, degrades pomalidomide-CRBN neosubstrates and suppresses cancer cell proliferation.

### Example 6

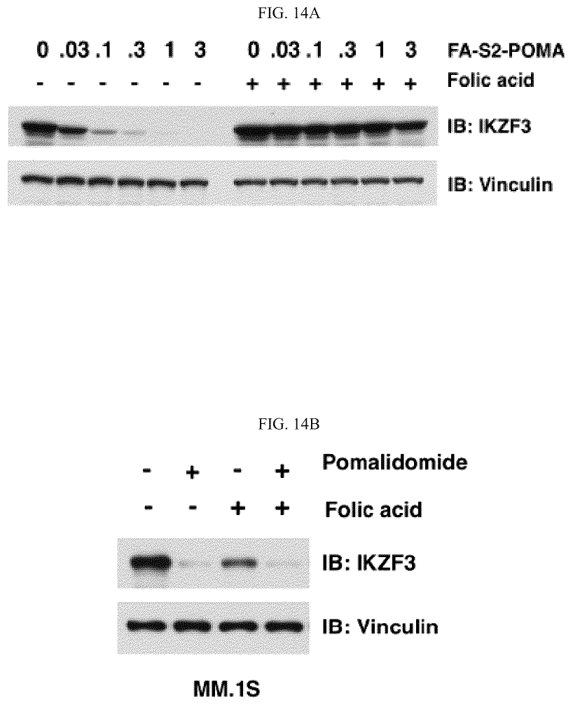

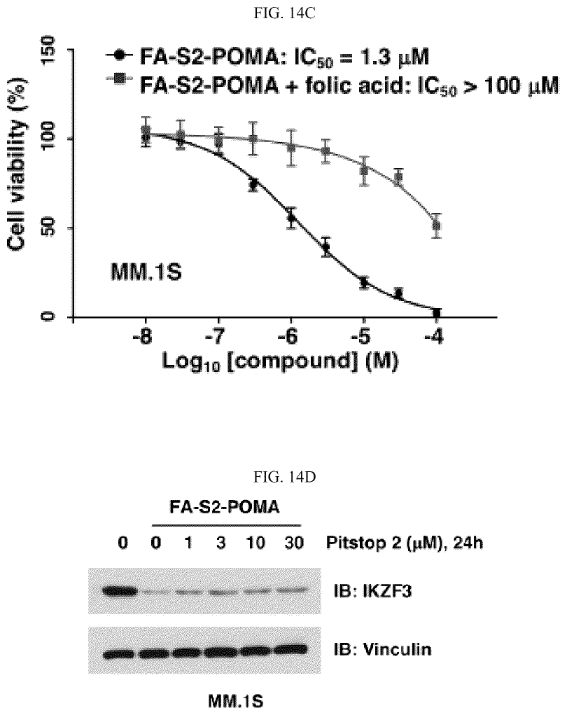

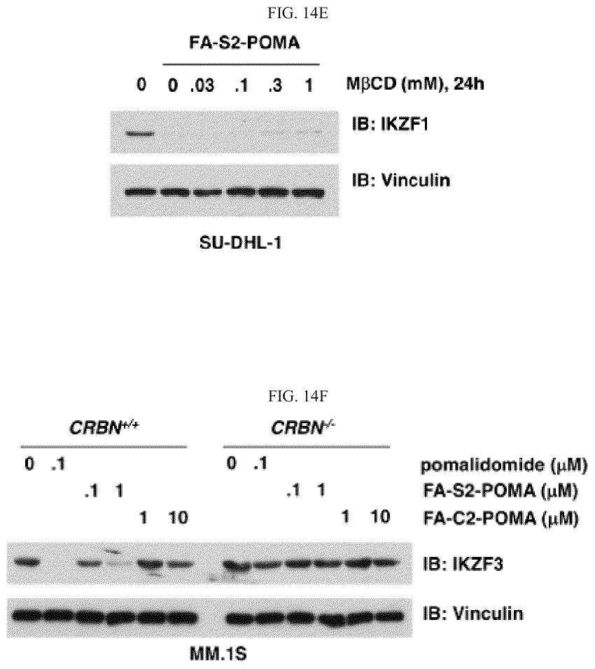

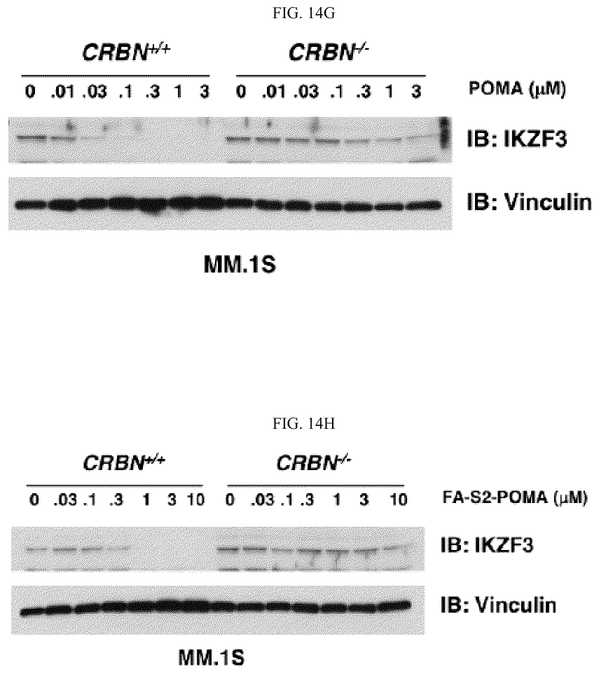

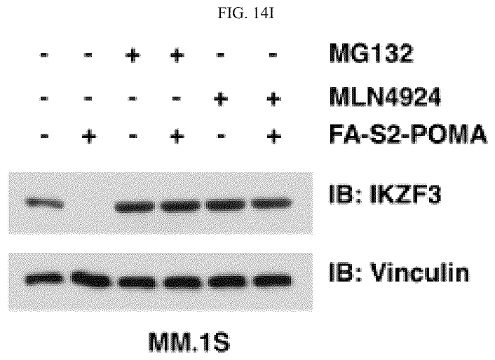

MM.1S cells were co-treated with FA-S2-POMA and 2.5 mM folic acid to saturate FOLR1 binding in order to assess the folate targeting group on FOLR1 expression. Notably, folic acid effectively antagonized the effect of FA-S2-POMA (FIG. 14A), but not pomalidomide (FIG. 14B) on degrading IKZF3 in MM.1S cells. Consistent with this result, co-treatment with folic acid led to a significant reduction in potency of FA-S2-POMA for suppression of the proliferation of MM.1S cells (FIG. 14C). Similarly, co-treatment with the endocytosis inhibitor pitstop 2 or methyl-β-cyclodextrin (MβCD) compromised the effect of FA-S2-POMA on degrading IKZFs in this experimental setting (FIGS. 14D-E). Collectively, these results suggest that FOLR1 and sequential endocytosis are important for FA-S2-POMA entry into cells.

Furthermore, depletion of the endogenous CRBN E3 ubiquitin ligase completely abolished the effect of pomalidomide and FA-S2-POMA on degrading IKZF3 in MM.1S cells (FIGS. 14F-H), indicating that the IKZF3 degradation effect of FA-S2-POMA depends on the CRBN E3 ubiquitin ligase.

MM.1S cells were also treated with FA-S2-POMA with or without a proteasome inhibitor (MG132-Benzyl N-[(2S)-4-methyl-1-[[(2S)-4-methyl-1-[[(2S)-4-methyl-1-oxopentan-2-yl]amino]-1-oxopentan-2-yl]amino]-1-oxopentan-2-yl]carbamate) or a Cullin-RING E3 ligase (CRL) neddylation inhibitor (MLN4924-[(1S,2S,4R)-4-[4-[[(1S)-2,3-Dihydro-1H-inden-1-yl]amino]-7H-pyrrolo[2,3-d]pyrimidin-7-yl]-2-hydroxycyclopentyl]methyl sulfamic acid ester). Both inhibitors blocked the effect of FA-S2-POMA on degradation of IKZF3 (FIG. 14I).

### Example 7

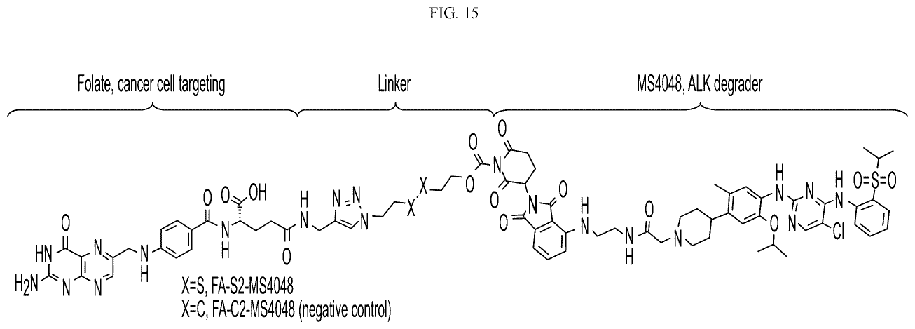

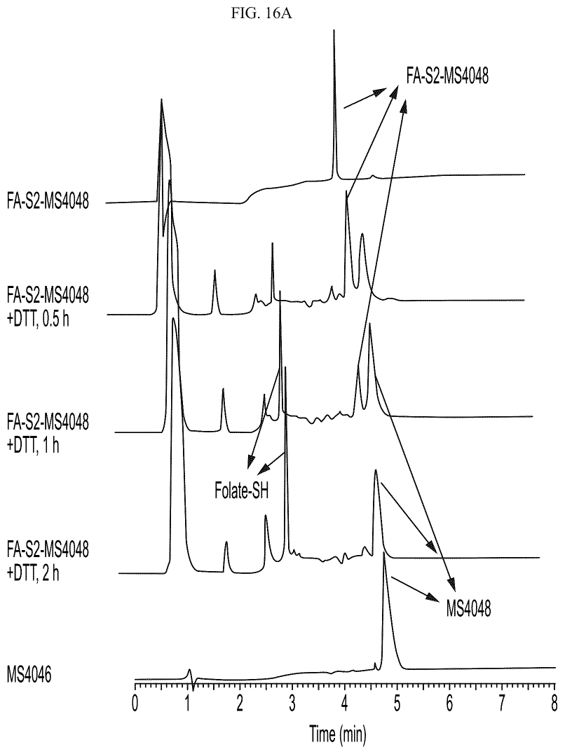

A folate-caged PROTAC, FA-S2-MS4048 (Compound 4), and its negative control FA-C2-MS4048 were synthesized and prepared as discussed below. The structure of the PROTAC and its negative control is shown in FIG. 15. MS4048 is an IMiD-based ALK PROTAC which may degrade ALK fusion proteins as discussed in Zhang, C et al Eur J Med Chem 151 (2018): 304-314 which is hereby incorporated by reference in its entirety and particularly in relation to MS4048. The folate-S2-(comprising disulfide moiety) and folate-C2-(replacing the disulfide with carbon-carbon) linkers were conjugated to the glutarimide nitrogen of the pomalidomide moiety in MS4048.

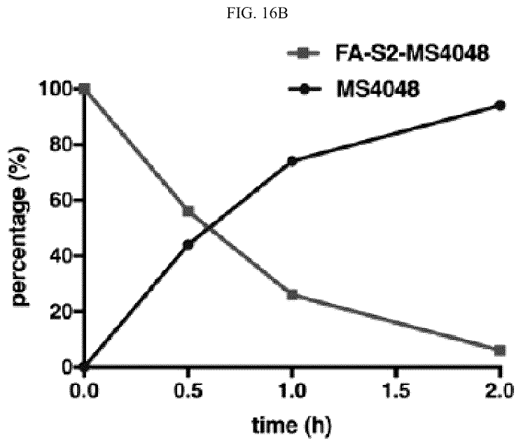

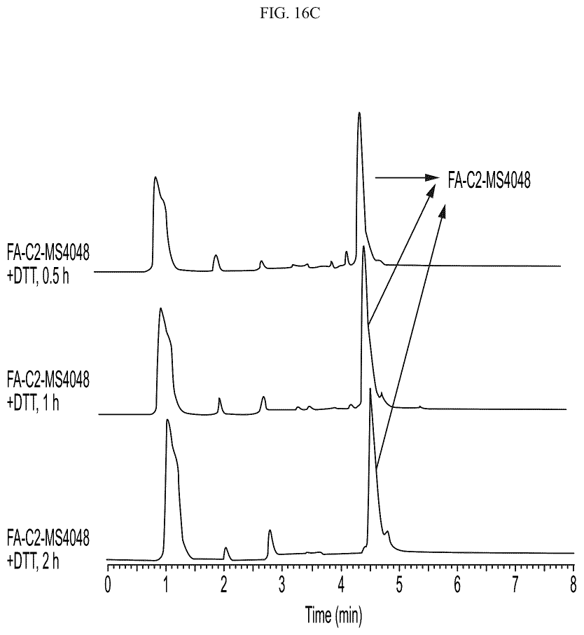

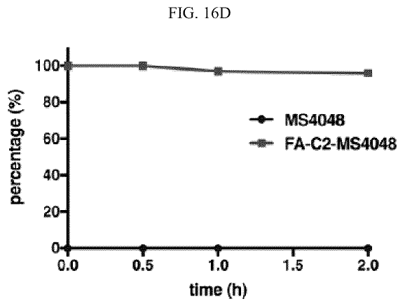

The effects of these folate caged PROTACS on ALK fusion protein degradation was assessed in cancer cells. FA-S2-MS4048 and FA-C2-MS4048 were incubated with DTT at 37° C. in PBS. It was found that the DTT treatment led to efficient release of MS4048 from FA-S2-MS4048 (FIGS. 16A and 16B), but not from FA-C2-POMA (FIGS. 16C and 16D). Furthermore, compared to FA-S2-POMA (FIG. 11B), the release rate of FA-S2-MS4048 is slower.

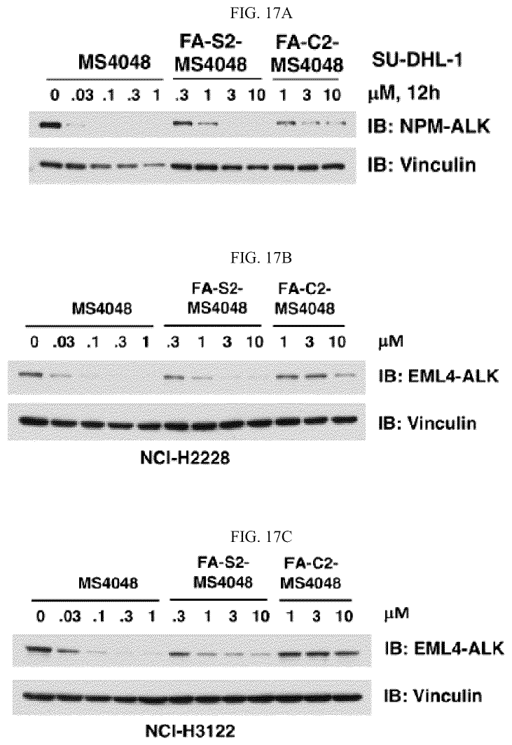

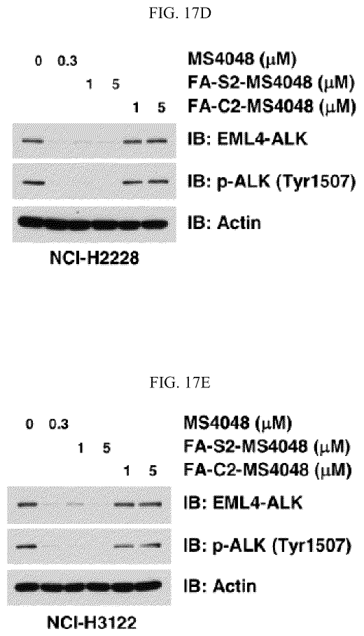

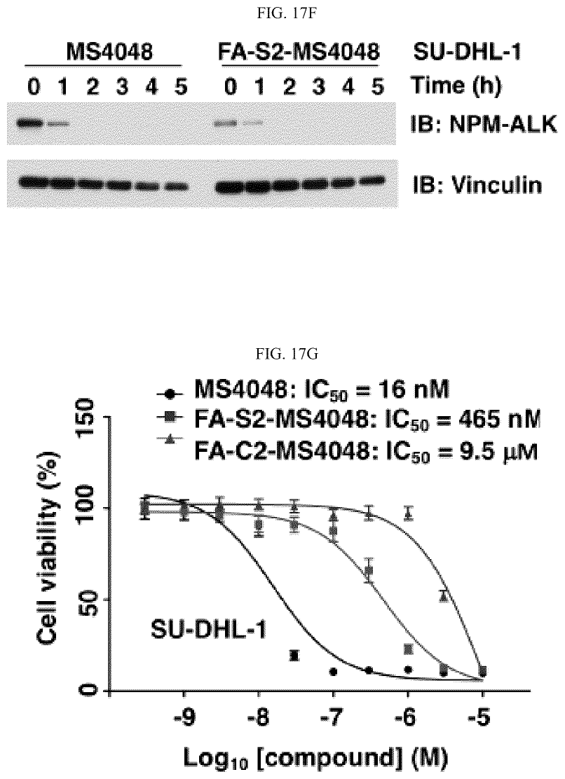

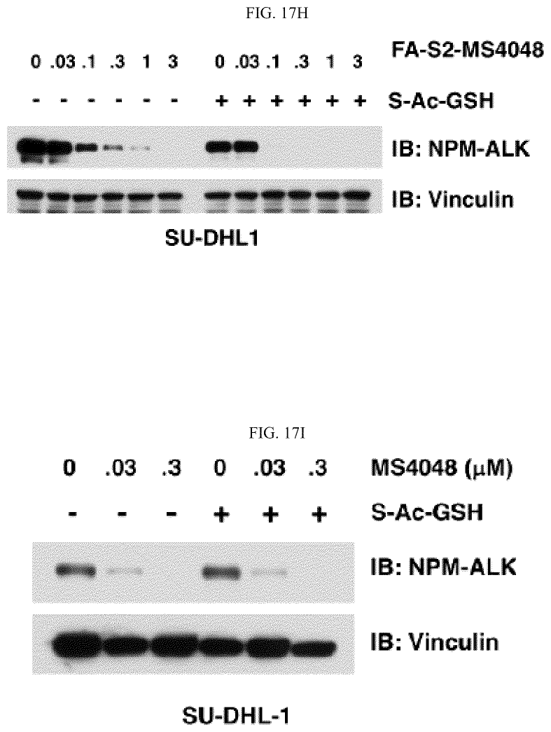

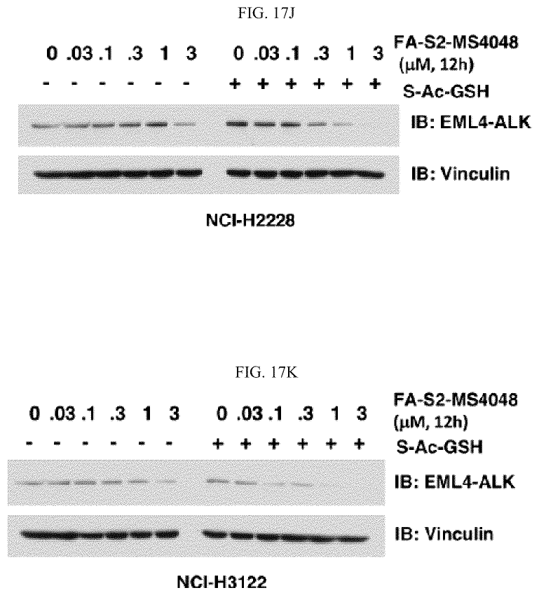

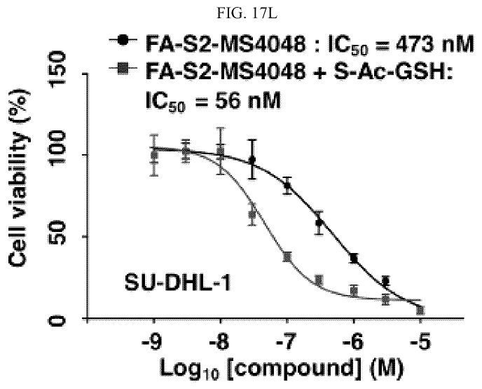

The effect of FA-S2-MS4048 on SU-DHL-1 cells, an anaplastic large cell lymphoma (ALCL) cell line harboring NPM-ALK fusion protein (Soda, M. et al Nature 448 (2007): 561-566, which is hereby incorporated by reference in its entirety), and two non-small cell lung cancer cell (NSCLC) cell lines harboring EML4-ALK fusion proteins (NCI-H2228 and NCI-H3122). FA-S2-MS4048 effectively degraded NPM-ALK in SU-DHL-1 cells (FIG. 17A) and EML4-ALK in NCI-H2228 and NCI-H3122 cells (FIGS. 17B-E) in a concentration and time dependent manner.

FA-S2-MS4048 may be less potent than its uncaged analog, MS4048, in this experiment, but it is still effective at degrading the proteins of interest. FA-S2-MS4048 is also more potent than FA-C2-MS4048 in degrading the ALK proteins. As can be seen, FA-C2-MS4048, unlike FA-C2-POMA, had some degradation effect as well. One possible explanation for this activity is the carbamate group in FA-C2-MS4048 is also hydrolysable and the rate of hydrolysis is dependent on cellular context.

The time dependency of FA-S2-MS4048 mediated degradation is illustrated in FIG. 17F. Significant degradation was observed as early as 1 hour for FA-S2-MS4048 administration. Additionally, it was determined that FA-S2-MS4048 effectively inhibited the proliferation of SU-DHL-1 cells with an IC₅₀ of 465 nM (FIG. 17G). The targeting group caged PROTAC may be less ptent than MS4048 (IC₅₀=16 nM), but more potent than FA-C2-MS4048 (IC₅₀=9.5 μM). The anti-proliferative effect of FA-S2-MS4048, MS4048 and FA-C2-MS4048 in SU-DHL-1 cells in consistent with their effect on degrading the NPM-ALK fusion protein.

Similar to the results obtained for FA-S2-POMA, pretreatment with S-acetyl-glutathione (S—Ac-GSH) to supplement the intracellular GSH level led to a significant increase in potency of FA-S2-MS4048 (FIG. 17H), but not MS4048 (FIG. 17I) in degrading NPM-ALK in SU-DHL-1 cells. Similar results in effective degradation of EML4-ALK in NCI-H23228 and NCI-H3122 cells (FIGS. 17J and 17K). Furthermore, pretreatment with S—Ac-GSH significantly increased potency of FA-S2-MS4048 in inhibiting the proliferation of SU-DHL-1 cells (FIG. 17L).

### Example 8

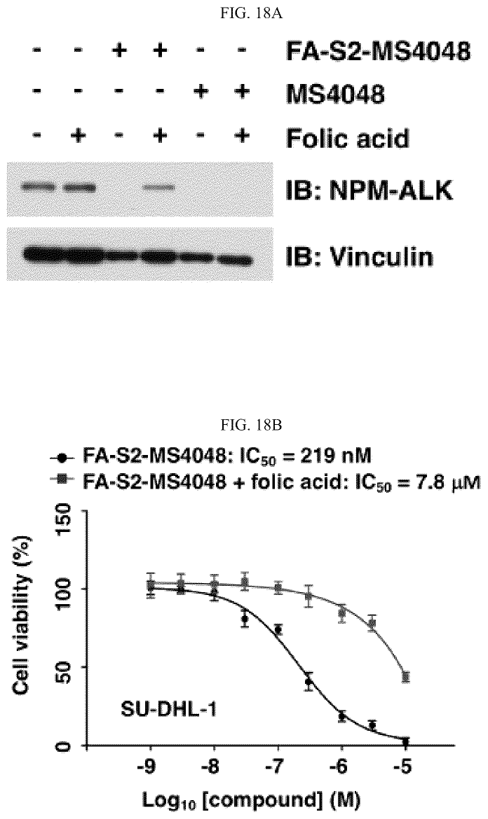

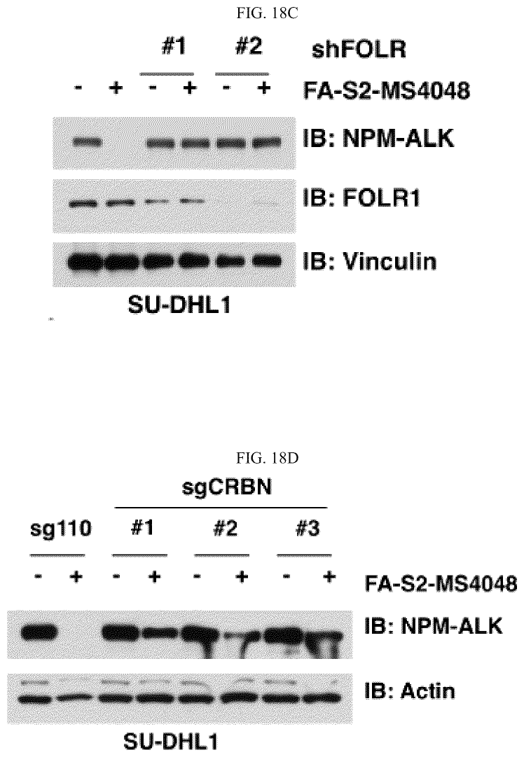

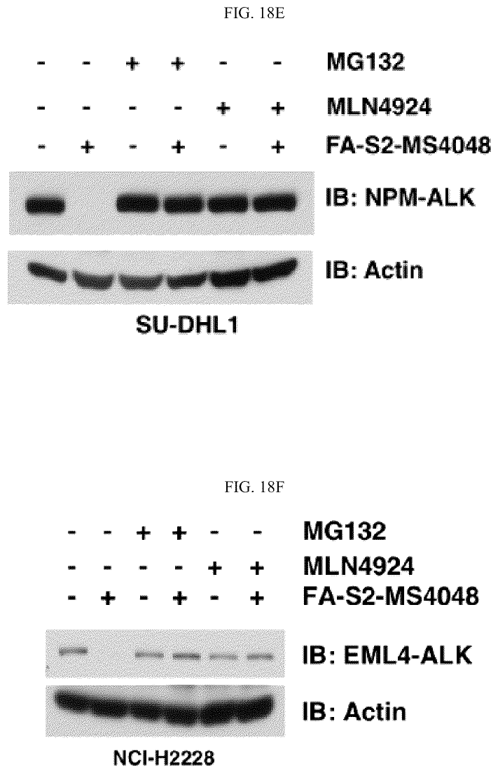

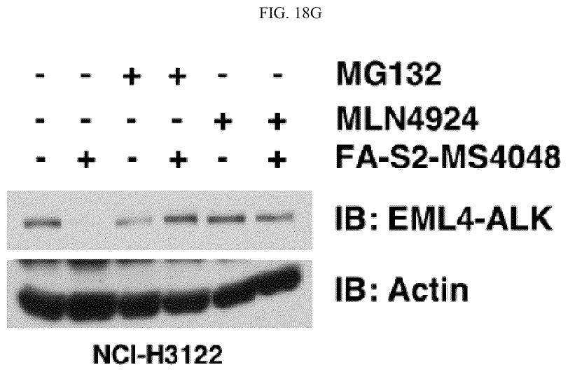

Pretreatment experiments were also performed with folic acid to assess the targeted delivery of FA-S2-MS4048. Pretreatement with folic acid indeed blocked the effect of FA-S2-MS4048, but not of MS4048, on degrading the NPM-ALK fusion protein in SU-DHL-a cells (FIG. 18A). Consistent with this result, the pretreatment with folic acid on SU-DHL-1 cells also significantly reduced potency of FA-S2-MS4048 in inhibiting the proliferation of S-DHL-1 cells (FIG. 18B).

Importantly, knockdown of FOLR1 via shRNAs completely abolished the effect of FA-S2-MS4048 on degrading NPM-ALK fusion protein in SU-DHL-1 cells (FIG. 18C). In addition, depletion of endogenous CRBN E3 ligase also significantly reduced the effect of FA-S2-MS4048 on degrading NPM-ALK in SU-DHL-1 cells (FIG. 18D) and inhibition of the proteasome by MG132 or inhibition of CRL neddylation by MLN4924 also blocked the degradation of NPM-ALK in SU-DHL1 cells (FIG. 18E) and EML4-ALK in NCI-H2228 (FIG. 18F) and NCI-H3122 (FIG. 18G). These results evidence that the folate-caged pomalidomide-derived ALK PROTAC, FA-S2-MS4048, degrades ALK fusion proteins in a FOLR1-, CRBN-, and proteasome-dependent manner.

Taken together, these results (Examples 1-8) demonstrate a cell membrane-targeting (particularly FOLR1-targeting) delivery strategy for PROTACs to selectively degrade POIs in cancer cells as compared to healthy cells. These results validate folate-PROTACs (Folate-ARV-771, Folate-MS432 and Folate-MS99, FA-S2-MS4048) that effectively degraded BRDs, MEK1/2 and ALK fusion proteins, respectively, in a FOLR1-dependent manner (in cancer cells). These results also demonstrate that this approach is generalizable and could be applied to all PROTACs, thereby providing a targeting strategy to selectively degrade proteins of interest in cancer cells and minimize potential toxicity/side-effects in normal tissues and/or cells, thus enhancing therapeutic windows of PROTACs. Moreover, this generalizable platform may be used, for example, no IMiD based molecular glues to cancer cells having higher membrane expression levels of certain proteins such as FOLR1. The platform may circumvent potential toxicity of these compounds.

### Materials and Methods: Biology

### Plasmids and Chemicals

shRNA for FOLR1 (TRCN0000372330) and folic acid (F8758) was purchased from Sigma. MβCD (#21633) was purchased from Cayman.

### Cell Culture

Human embryonic kidney 293T (HEK293T), HFF-1 (human normal fibroblast), HeLa, OVCAR8, MDA-MB-231, MDA-MB-468, CAMA-1, BT474, SK-MEL-28 cells were maintained in Dulbecco's Modified Eagle's Medium (DMEM) containing 10% fetal bovine serum (FBS), 100 Units of penicillin and 100 μg/ml streptomycin. MCF10A cells were maintained in Dulbecco's Modified Eagle's Medium (DMEM)/Ham's F12 (CellGro) supplemented with 5% equine serum (CellGro), 10 μg/mL insulin (Life Technologies), 500 ng/mL hydrocortisone (Sigma-Aldrich), 20 ng/mL epidermal growth factor (EGF) (R&D Systems), and 100 ng/mL cholera toxin (Sigma-Aldrich). T47D, BT549, HCC-1937, ZR-75-1, HCC1428, AU565, HCC1954, ZR-75-30 SU-DHL1, NCI-H2228, NCI-H3122 and HT-29 cells were cultured in RPMI1640 containing 10% fetal bovine serum (FBS), 100 Units of penicillin and 100 μg/ml streptomycin. were cultured in MEM containing 10% fetal bovine serum (FBS), 100 Units of penicillin and 100 μg/ml streptomycin. SK-BR-3 cells were cultured in McCoy's 5A containing 10% fetal bovine serum (FBS), 100 Units of penicillin and 100 μg/ml streptomycin. The shRNA lentivirus was generated in HEK293T cells for the infection of HeLa and T47D cells.

Human embryonic kidney 293T (HEK293T), MM.1S cells were maintained in Dulbecco's ModifiedEagle's Medium (DMEM) containing 10% fetal bovine serum (FBS), 100 Units of penicillin and 100 μg/ml streptomycin. SU-DHL1, NCI-H2228 and NCI-H3122 cells were cultured in RPMI1640 containing 10% FBS, 100 Units of penicillin and 100 μg/ml streptomycin. The usage MM.1S cells forevaluation of pomalidomide, as well as the usage of SU-DHL-1, NCI-H2228 and NCI-H3122 cells for evaluation of ALK degrader is based on our previous reports. MM.1S-CRBN^{+/+} and MM.1S-CRBN^{-/-} cells were kindly gift from Dr. William Kaelin, Jr. at Dana-Farber Cancer Institute, Harvard Medical School. SU-DHL-1-CRBN^{-/-} cells were generated using CRISPR-Cas9 technology with sgRNA as below: sg #1: TAAACAGACATGGCCGGCGA (SEQ ID NO: 1), sg #2: GTCCTGCTGATCTCCTTCGC (SEQ ID NO: 2), sg #3: CAGGACGCTGCGCACAACAT (SEQ ID NO: 3). The lentivirus of sgCRBN was generated in HEK293T cells for the infection of SU-DHL-1 cells as previously described. Cells were infected with lentivirus, selected with puromycin for 72 hours, followed by further PROTAC treatment.

For folic acid competitive degradation assay, cells were pretreated with folic acid (F8758, Sigma-Aldrich, 2.5 mM) for 2 hours, followed by the treatment of indicated PROTACs for indicated time. For endocytosis inhibition, cells were pretreated with Pitostop 2 (SML1169, Sigma-Aldrich, 1-30 μM) or MCD (21633, Cayman, 0.03-1 mM) for 12 hours, followed by co-treatment with indicated PROTACs for another 12 hours. For proteasome or E3 ligase inhibition assay, cells were treated with MG132 (BML-P11020, ENZO Life Sciences, 10 μM) or MLN4924 (S7109, SelleckChem, 1 μM) another 12 hours. For GSH-stimulating assay, cells were treated with S-Acetyl-GSH (29624, Cayman, 1 mM) with indicated PROTACs for 12 or 72 hours.

### Antibodies

Anti-BRD3 (11859-1-AP) antibody was purchased from Proteintech. Anti-BRD4 (A301-985A-M) antibody was purchased from Bethyl Laboratories. Anti-ALK (3633), MEK1 (2352) and MEK2 (9147) antibodies were purchased from Cell Signaling Technologies. Monoclonal anti-vinculin antibody (V-4505), peroxidase-conjugated anti-mouse secondary antibody (A-4416) and peroxidase-conjugated anti-rabbit secondary antibody (A-4914) were purchased from Sigma. All antibodies were used at a 1:1,000 dilution in 5% bovine serum albumin (BSA) in TBST buffer for western blots.

Anti-IKZF1 (ab191394, 1:1,000) antibody was purchased from Abcam. Anti-IKZF3 (NBP22449, 1:1,000) antibody was purchased from Novus Biologicals. Anti-ALK (3633, 1:1,000) was purchased from Cell Signaling Technologies. Monoclonal anti-Flag antibody (F-3165, 1:5,000), anti-vinculin antibody (V-4505; 1:50,000), peroxidase-conjugated anti-mouse secondary antibody (A-4416; 1:5,000), and peroxidase-conjugated anti-rabbit secondary antibody (A-4914; 1:5,000) were purchased from Sigma-Aldrich. All primary antibodies were prepared in 5% bovine serum albumin (BSA) in TBST buffer and secondary antibodies were prepared in 5% non-fat milk in TBST buffer.

### Immunoblots (IB) Assay

Cells were lysed in EBC buffer (50 mM Tris pH 7.5, 120 mM NaCl, 0.5% NP-40) supplemented with protease inhibitors (Pierce) and phosphatase inhibitors (phosphatase inhibitor cocktail set I and II, Calbiochem). The protein concentrations of the lysates were measured using the Bio-Rad protein assay reagent on a Beckman Coulter DU-800 spectrophotometer. The lysates were then resolved by SDS-PAGE and immunoblotted with indicated antibodies.

### CCK-8 Cell Proliferation Assay

Cell in 96-well plates were treated with drugs as indicated, and then incubated with 10 μl/well of CCK-8 (cell counting Kit-8) solution at for 1-2 hours, followed by the measurement of optical density at 450 nm.

### Streptavidin Pulldown Assays

The streptavidin pulldown assay for biotin-pomalidomide was performed as previous reported. Briefly, Flag-CRBN was expressed in HEK293T cells lysed in PROTAC buffer B (50 mM Tris-HCl pH7.5, 150 mM NaCl and 0.5% NP-40) supplemented with protease inhibitors (Pierce) and phosphatase inhibitors (phosphatase inhibitor cocktail set I and II, Calbiochem). A total of 1 mg of cell lysate was incubated with 10 μl of 10 mM biotin-pomalidomide and 8 μl of streptavidin beads for 1 hour at 4° C. in the absence or presence of pomalidomide or its derivates. Alternatively, Folate-S2-pomalidomide or Folate-C2-pomalidomide were incubated with 5 mM GSH and then subjected to the competition bindingassay. Then, the beads were washed four times with NETN buffer (20 mM Tris-HCl pH 7.5, pH 8.0, 100 mM NaCl, 1 mM EDTA and 0.5% NP-40), before being resolved by SDS-PAGE and immunoblotted with Flag-tag antibody.

### Western Blot Assy

Cells were lysed in EBC buffer (50 mM Tris-HCl pH 7.5, 120 mM NaCl and 0.5% NP-40) supplemented with protease inhibitors (Pierce) and phosphatase inhibitors (phosphatase inhibitor cocktailset I and II, Calbiochem). The protein concentrations of the lysates were measured using the Bio-Rad protein assay reagent on a Beckman Coulter DU-800 spectrophotometer. The lysates (40 μg protein) werethen resolved by 10% SDS-olyacrylamide gel electrophoresis (PAGE) at 130 V for 80 min and immunoblotted with indicated antibodies at 4° C. overnight, washed four times with Tris-buffered saline with 0.1% Tween-20 (TBST) buffer, incubated with secondary antibody for 1 hour at room temperature, and then washed 4 times with TBST. All western blot assays were repeated at least twice independently, and one representative result were shown in figures.

### Materials and Methods: Chemistry

Common reagents or materials were purchased from commercial sources and used without further purification. Ultra-performance liquid chromatography (UPLC) spectra for compounds were acquired using a Waters Acquity I-Class UPLC system with a PDA detector. Chromatography was performed on a 2.1 Å˜, 30 mm ACQUITY UPLC BEH C18 1.7 μm column with water containing 3% acetonitrile, 0.1% formic acid as solvent A and acetonitrile containing 0.1% formic acid as solvent B at a flow rate of 0.8 mL/min. The gradient program was as follows: 1-99% B (1-1.5 min), and 99-1% B (1.5-2.5 min). High-performance liquid chromatography (HPLC) spectra were acquired using an Agilent 1200 Series system with DAD detector for all the intermediates and final products below. Chromatography was performed on a 2.1×150 mm Zorbax 300SB-C18 5 μm column with water containing 0.1% formic acid as solvent A and acetonitrile containing 0.1% formic acid as solvent B at a flow rate of 0.4 ml/min. The gradient program was as follows: 1% B (0-1 min), 1-99% B (1-4 min), and 99% B (4-8 min). High-resolution mass spectra (HRMS) data were acquired in positive ion mode using an Agilent G1969AAPI-TOF with an electrospray ionization (ESI) source. Nuclear Magnetic Resonance (NMR) spectra were acquired on a Bruker DRX-600 spectrometer with 600 MHz for proton (¹H NMR) and 151 MHz for carbon (¹³C NMR); chemical shifts are reported in (δ). Preparative HPLC was performed on Agilent Prep 1200 series with UV detector set to 220 or 254 nm. Samples were injected onto a Phenomenex Luna 250×30 mm, 5 μm, C₁₈ column at room temperature. The flow rate was 40 ml/min. A linear gradient was used with 10% of acetonitrile in H₂O (with 0.1% TFA) (B) to 100% of acetonitrile (A). HPLC was used to establish the purity of target compounds. All final compounds had >96% purity using the HPLC methods described above. ARV-771, MS432 Ac-VHL, and MS4048 were synthesized according to the published procedures.

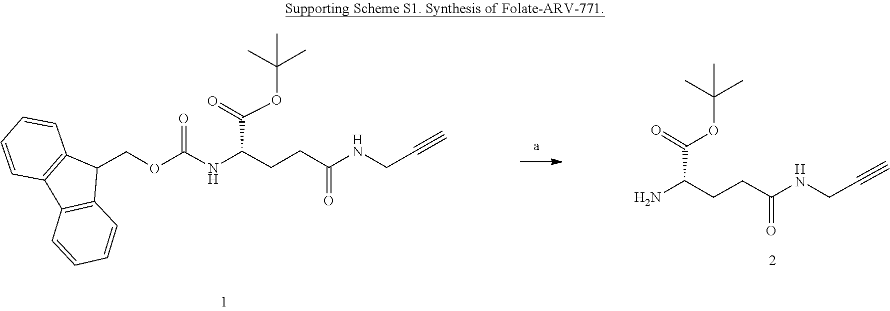

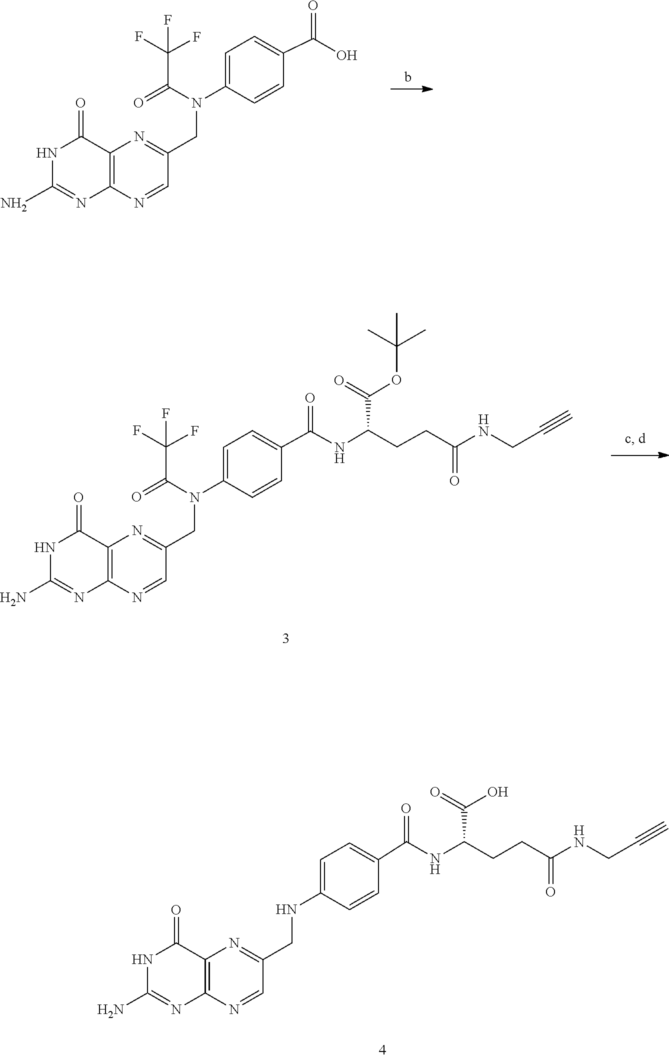 [SMILES: Nc1nc2ncc(CN(C(=O)C(F)(F)F)c3ccc(C(=O)O)cc3)nc2c(=O)[nH]1 ; C#CCNC(=O)CC[C@H](NC(=O)c1ccc(N(Cc2cnc3nc(N)[nH]c(=O)c3n2)C(=O)C(F)(F)F)cc1)C(=O)OC(C)(C)C ; C#CCNC(=O)CC[C@H](NC(=O)c1ccc(NCc2cnc3nc(N)[nH]c(=O)c3n2)cc1)C(=O)O]

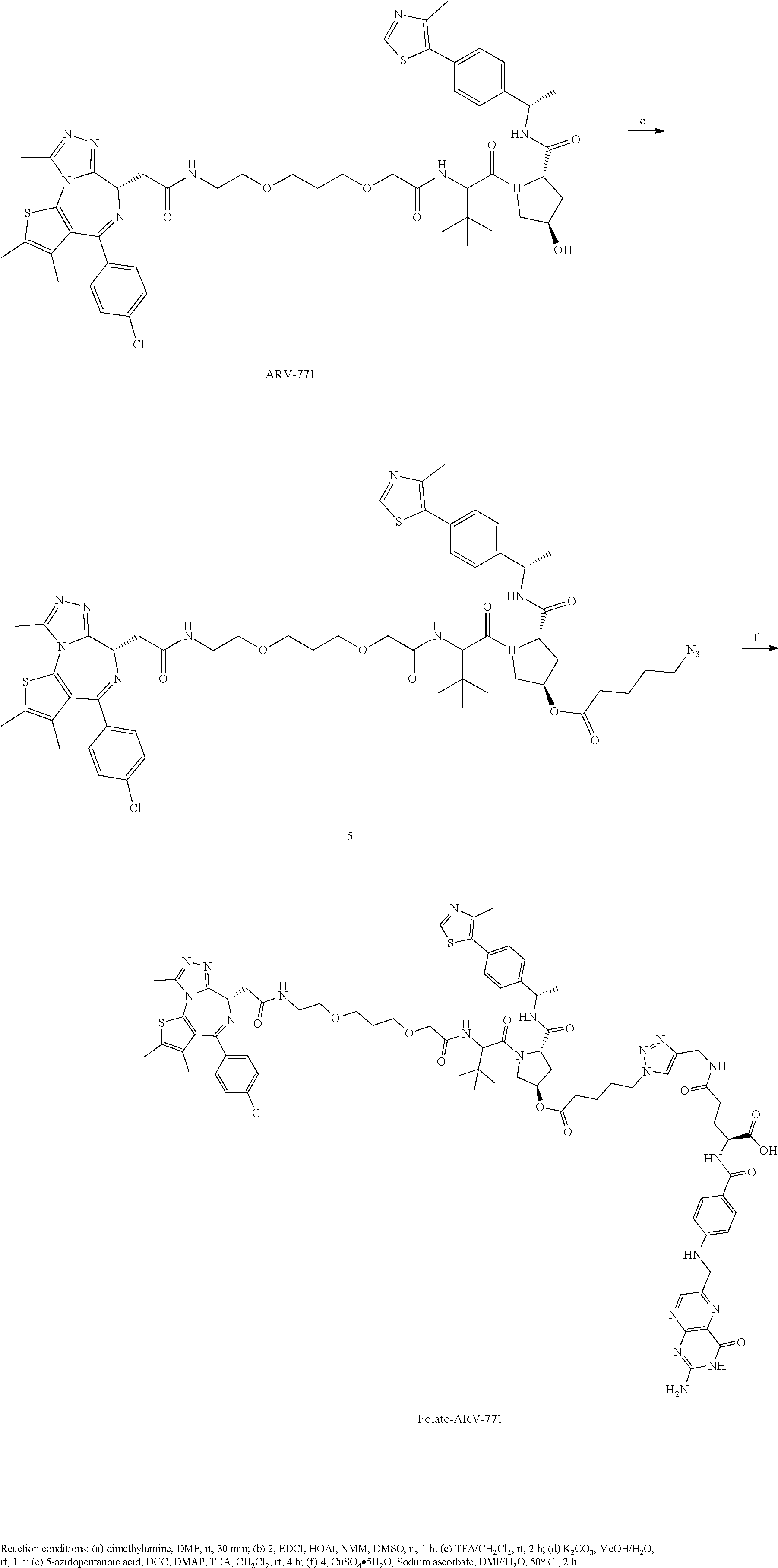

Synthesis of tert-butyl N⁵-(prop-2-yn-1-yl)-L-glutaminate (2)

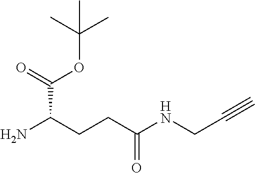 [SMILES: C#CCNC(=O)CC[C@H](N)C(=O)OC(C)(C)C]

A similar synthesis, or synthesis of an intermediate is described in Candelon, N. Chem. Commun. 49 (2013): 9206-9208, which is hereby incorporated by reference in its entirety. Briefly, to a solution of compound 1 (300 mg, 0.65 mmol, 1.0 equiv) in DMF (2 mL) was added dimethylamine (1.62 mL, 2M THF solution, 3.25 mmol, 5.0 equiv) at room temperature. The reaction mixture was stirred at room temperature for 30 min. Then the reaction solution was diluted with ethyl acetate (30 mL) and washed with water (2×30 mL) and brine (30 mL). The organic layer was concentrated under reduced pressure. The crude product was purified by preparative HPLC (10%-100% acetonitrile/0.1% TFA in H₂O) to afford compound 2 as colorless solid in TFA salt form (213 mg, 93%). ¹H NMR (600 MHz, Methanol-d₄) δ 4.05-3.93 (m, 3H), 2.59 (t, J=2.5 Hz, 1H), 2.54-2.42 (m, 2H), 2.24-2.09 (m, 2H), 1.54 (s, 9H). ¹³C NMR (151 MHz, Methanol-d₄) δ 172.18, 167.92, 84.03, 79.47, 70.88, 52.61, 30.59, 28.14, 26.73, 25.68. ESI m/z=241.3 [M+H]⁺.

Synthesis of tert-butyl N²-(4-(N-((2-amino-4-oxo-3,4-dihydropteridin-6-yl)methyl)-2,2,2-trifluoroacetamido)benzoyl)-N⁵-(prop-2-yn-1-yl)-L-glutaminate (3)

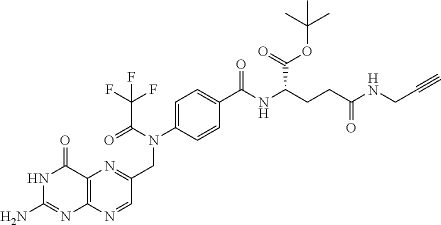 [SMILES: C#CCNC(=O)CC[C@H](NC(=O)c1ccc(N(Cc2cnc3nc(N)[nH]c(=O)c3n2)C(=O)C(F)(F)F)cc1)C(=O)OC(C)(C)C]

To a solution of N¹⁰-(Trifluoroacetyl)pteroic acid (204.2 mg, 0.5 mmol, 1.0 equiv) in DMSO (4 mL) were added compound 2 (212.6 mg, 0.6 mmol, 1.2 equiv), EDCI (1-ethyl-3-(3-dimethylaminopropyl)carbodiimide) (191.7 mg, 1.0 mmol, 2.0 equiv), HOAt (1-hydroxy-7-azabenzo-triazole) (136.1 mg, 1.0 mmol, 2.0 equiv) and NMM (N-Methylmorpholine) (202.2 mg, 2.0 mmol, 4.0 equiv). After being stirred at room temperature for 1 h, the resulting mixture was purified by preparative HPLC (10%-100% acetonitrile/0.1% TFA in H₂O) to afford compound 3 as brown solid (258.5 mg, 82%). ¹H NMR (600 MHz, DMSO-d₆) δ 8.77 (d, J=7.4 Hz, 1H), 8.73 (s, 1H), 8.31 (t, J=5.5 Hz, 1H), 7.92 (d, J=8.2 Hz, 2H), 7.66 (d, J=8.1 Hz, 2H), 5.18 (s, 2H), 4.31-4.22 (m, 1H), 3.92-3.78 (m, 2H), 3.07 (t, J=2.5 Hz, 1H), 2.34-2.19 (m, 2H), 2.08-2.02 (m, 1H), 1.98-1.88 (m, 1H), 1.41 (s, 9H). ¹³C NMR (151 MHz, DMSO-d₆) δ 171.13, 171.02, 165.71, 159.88, 155.72 (q, J=34.7 Hz), 153.27, 152.31, 148.95, 145.91, 141.62, 134.54, 128.70, 128.49, 128.13, 116.12 (q, J=288.4 Hz), 81.19, 80.68, 72.91, 53.82, 53.16, 31.49, 27.84, 27.68, 26.21. ESI m/z=631.3 [M+H]⁺.

Synthesis of N²-(4-(((2-amino-4-oxo-3,4-dihydropteridin-6-yl)methyl)amino)benzoyl)-N⁵-(prop-2-yn-1-yl)-L-glutamine (4)

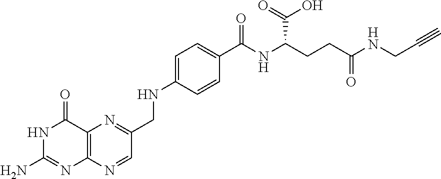 [SMILES: C#CCNC(=O)CC[C@H](NC(=O)c1ccc(NCc2cnc3nc(N)[nH]c(=O)c3n2)cc1)C(=O)O]

To a suspension of compound 3 (258.5 mg, 0.41 mmol) in dichloromethane (2 mL) was added TFA (trifluoroacetic acid) (2 mL) at room temperature. After being stirred at room temperature for 2 h, the resulting mixture was concentrated and purified by preparative HPLC (10%-100% acetonitrile/0.1% TFA in H₂O) to afford the intermediate as brown solid. ESI m/z=575.3 [M+H⁺]. To a suspension of the obtained brown solid in methanol (3 mL) was dropwise added a solution of K₂CO₃ (169.7 mg) in water (1 mL) at room temperature. The reaction mixture was stirred at room temperature for 1 h and then concentrated under reduced pressure to remove methanol. The resulting solution was diluted with water (3 mL) and then pH was adjusted with hydrochloric acid (HCl, 3N) to 2-3. The suspension was filtered and the solid cake was washed with water. After being dried under reduced pressure, afforded compound 4 as brown solid (132.0 mg, 67% yield for two step). ¹H NMR (600 MHz, DMSO-d₆) δ 12.46 (s, 1H), 11.41 (s, 1H), 8.65 (s, 1H), 8.27 (t, J=5.5 Hz, 1H), 8.16 (d, J=7.6 Hz, 1H), 7.65 (d, J=8.8 Hz, 2H), 6.93 (s, 1H), 6.64 (d, J=8.8 Hz, 2H), 4.52-4.45 (m, 2H), 4.27 (ddd, J=9.8, 7.5, 4.7 Hz, 1H), 3.88-3.77 (m, 2H), 3.06 (t, J=2.5 Hz, 1H), 2.26-2.15 (m, 2H), 2.10-1.99 (m, 1H), 1.95-1.84 (m, 1H). ¹³C NMR (151 MHz, DMSO-d₆) δ 174.27, 171.82, 166.83, 161.53, 156.55, 154.25, 151.23, 149.14, 149.05, 129.46, 128.41, 121.80, 111.66, 81.68, 73.34, 52.63, 46.37, 32.18, 28.29, 26.87. ESI m/z=479.4 [M+H]⁺.

Synthesis of (3R,5S)-1-((S)-2-(tert-butyl)-15-((S)-4-(4-chlorophenyl)-2,3,9-trimethyl-6H-thieno[3,2-f][1,2,4]triazolo[4,3-a][1,4]diazepin-6-yl)-4,14-dioxo-6,10-dioxa-3,13-diazapentadecanoyl)-5-(((S)-1-(4-(4-methylthiazol-5-yl)phenyl)ethyl)carbamoyl)pyrrolidin-3-yl 5-azidopentanoate (5)

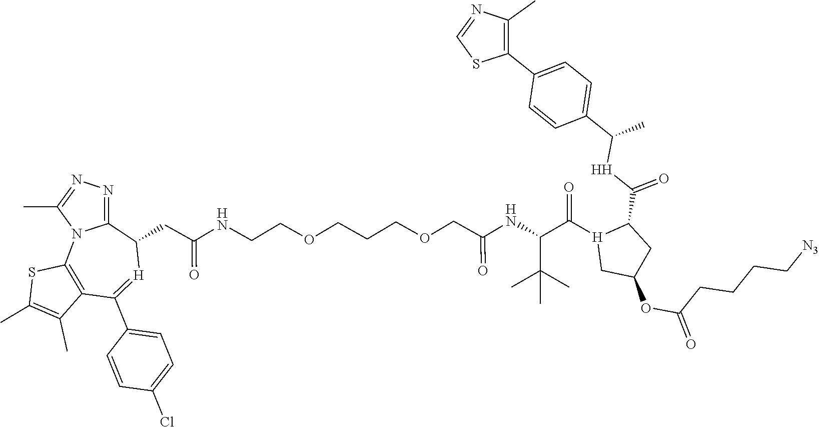

A similar synthesis, or synthesis of intermediates is described in Raina, K. Proc. Natl. Acad. Sci. 113 (2016): 7124-7129, which is hereby incorporated by reference in its entirety. Briefly, to a solution of ARV771 (29.0 mg, 0.03 mmol, 1.0 equiv) in dichloromethane (2 mL) were added 5-azidopentanoic acid (8.6 mg, 0.06 mmol, 2.0 equiv), DCC (N,N′-Dicyclohexylcarbodiimide) (9.3 mg, 0.045 mmol, 1.5 equiv), DMAP (4-Dimethylaminopyridine) (0.4 mg, 0.003 mmol, 0.1 equiv) and TEA (triethylamine) (3 mg, 0.03 mmol, 1.0 equiv). After being stirred at room temperature for 4 h, the resulting mixture was purified by preparative HPLC (10%-100% acetonitrile/0.1% TFA in H₂O) to afford compound 5 as white solid (23.0 mg, 69%). ¹H NMR (600 MHz, Methanol-d₄) δ 9.19 (s, 1H), 7.53-7.41 (m, 8H), 5.35 (t, J=4.3 Hz, 1H), 5.02 (q, J=7.1, 6.7 Hz, 1H), 4.73 (dd, J=8.8, 5.2 Hz, 1H), 4.67-4.58 (m, 2H), 4.13 (d, J=12.0 Hz, 1H), 4.06-3.96 (m, 2H), 3.90 (dd, J=11.9, 4.0 Hz, 1H), 3.74-3.63 (m, 4H), 3.62 (t, J=5.4 Hz, 2H), 3.55-3.45 (m, 3H), 3.41-3.36 (m, 1H), 3.30 (t, J=6.6 Hz, 2H), 2.77 (s, 3H), 2.52 (s, 3H), 2.47 (s, 3H), 2.43-2.35 (m, 3H), 2.14 (ddd, J=14.0, 9.4, 4.7 Hz, 1H), 1.97-1.90 (m, 2H), 1.71 (s, 3H), 1.69-1.63 (m, 2H), 1.61-1.56 (m, 2H), 1.51 (d, J=7.0 Hz, 3H), 1.06 (s, 9H). ESI m/z=1111.3 [M+H]⁺.

Synthesis of N²-(4-(((2-amino-4-oxo-3,4-dihydropteridin-6-yl)methyl)amino)benzoyl)-N⁵-((1-(5-(((3R,5S)-1-((S)-2-(2-(3-(2-(2-((S)-4-(4-chlorophenyl)-2,3,9-trimethyl-6H-thieno[3,2-f][1,2,4]triazolo[4,3-a][1,4]diazepin-6-yl)acetamido)ethoxy)propoxy)acetamido)-3,3-dimethylbutanoyl)-5-(((S)-1-(4-(4-methylthiazol-5-yl)phenyl)ethyl)carbamoyl)pyrrolidin-3-yl)oxy)-5-oxopentyl)-1H-1,2,3-triazol-4-yl)methyl)-L-glutamine (Folate-ARV-771)

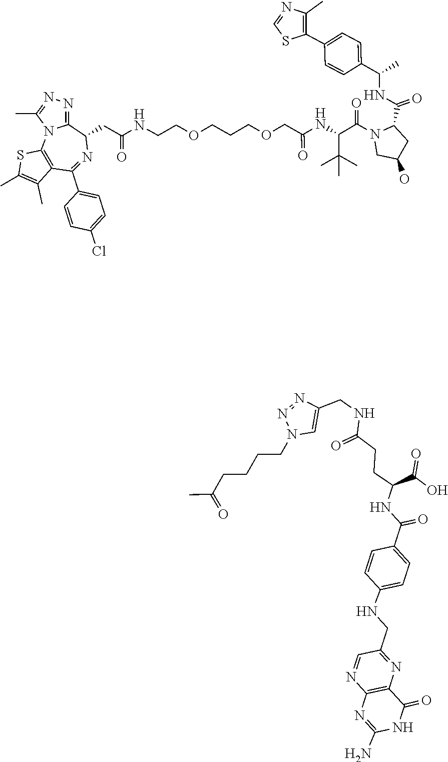 [SMILES: Cc1ncsc1-c1ccc([C@H](C)NC(=O)[C@@H]2C[C@@H](O)CN2C(=O)[C@@H](NC(=O)COCCCOCCNC(=O)C[C@@H]2N=C(c3ccc(Cl)cc3)c3c(sc(C)c3C)-n3c(C)nnc32)C(C)(C)C)cc1 ; CC(=O)CCCCn1cc(CNC(=O)CC[C@H](NC(=O)c2ccc(NCc3cnc4nc(N)[nH]c(=O)c4n3)cc2)C(=O)O)nn1]

To a solution of compound 5 (23.0 mg, 0.02 mmol, 1.0 equiv) in DMF (1.2 mL)/water (0.6 mL) were added compound 4 (12 mg, 0.024 mmol, 1.2 equiv), sodium ascorbate (1.6 mg, 0.008 mmol, 0.4 equiv) and CuSO₄·5H₂O (1.0 mg, 0.004 mmol, 0.2 equiv) at room temperature. The reaction mixture was heated to 50° C. After being stirred at 50° C. for 2 h, the resulting mixture was purified by preparative HPLC (10%-100% acetonitrile/0.1% TFA in H₂O) to afford Folate-ARV-771 as light yellow solid (16.1 mg, 51%). ¹H NMR (600 MHz, DMSO-d₆) δ 9.03 (s, 1H), 8.73 (s, 1H), 8.52 (d, J=7.7 Hz, 1H), 8.38 (t, J=5.6 Hz, 1H), 8.32 (t, J=5.7 Hz, 1H), 8.24 (d, J=7.6 Hz, 1H), 7.91 (s, 1H), 7.70 (d, J=8.4 Hz, 2H), 7.52 (d, J=8.3 Hz, 2H), 7.49-7.37 (m, 7H), 6.68 (d, J=8.4 Hz, 2H), 5.26 (s, 1H), 4.98-4.92 (m, 1H), 4.61-4.54 (m, 3H), 4.51 (t, J=8.4 Hz, 1H), 4.47 (d, J=9.4 Hz, 1H), 4.39-4.26 (m, 5H), 4.00-3.90 (m, 3H), 3.85-3.78 (m, 1H), 3.61-3.51 (m, 4H), 3.46 (t, J=6.0 Hz, 2H), 3.40-3.23 (m, 4H), 2.64 (s, 3H), 2.49 (s, 3H), 2.45 (s, 3H), 2.39-2.23 (m, 5H), 2.18-2.08 (m, 1H), 2.06-1.92 (m, 2H), 1.89-1.78 (m, 4H), 1.66 (s, 3H), 1.55-1.45 (m, 2H), 1.41 (d, J=7.0 Hz, 3H), 0.99 (s, 9H). ¹³C NMR (151 MHz, DMSO) δ 174.29, 172.74, 172.03, 170.22, 170.14, 169.68, 169.34, 166.80, 163.63, 160.03, 155.58, 153.19, 152.04, 151.63, 151.07, 150.81, 148.42, 148.14, 145.41, 145.06, 137.10, 135.79, 132.64, 131.66, 131.37, 130.65, 130.36, 130.16, 130.10, 129.46, 129.39, 129.33, 128.92, 128.42, 126.79, 123.10, 121.94, 111.73, 73.45, 69.82, 69.36, 68.47, 67.52, 58.71, 56.53, 54.23, 54.13, 52.58, 49.30, 48.35, 46.23, 39.08, 37.87, 35.59, 35.00, 34.72, 33.16, 32.27, 29.89, 29.41, 26.93, 26.61, 22.91, 21.64, 16.38, 14.49, 13.13, 11.73. HRMS (ESI-TOF) calcd for C₇₆H₉₀ClN₂₀O₁₃S₂⁺[M+H]⁺ 1589.6121, found 1589.6103. HPLC>98%, t_{R}=4.29 min.

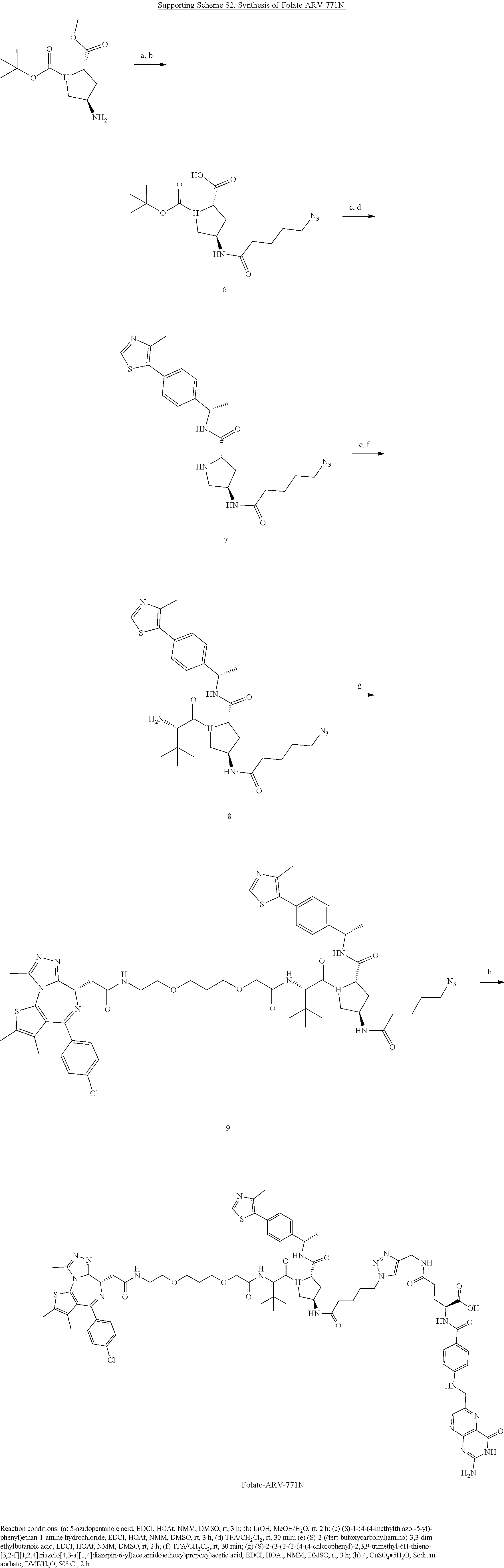

Synthesis of (2S,4R)-4-(5-azidopentanamido)-1-(tert-butoxycarbonyl)pyrrolidine-2-carboxylic acid (6)

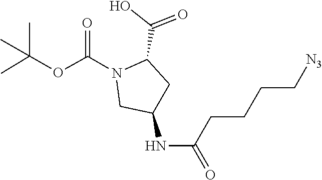 [SMILES: CC(C)(C)OC(=O)N1C[C@H](NC(=O)CCCCN=[N+]=[N-])C[C@H]1C(=O)O]

To a solution of 1-(tert-butyl) 2-methyl (2S,4R)-4-aminopyrrolidine-1,2-dicarboxylate (146.6 mg, 0.6 mmol, 1.5 equiv) in DMSO (3 mL) were added 5-azidopentanoic acid (57.3 mg, 0.4 mmol, 1.0 equiv), EDCI (153.4 mg, 0.8 mmol, 2.0 equiv), HOAt (108.8 mg, 0.8 mmol, 2.0 equiv) and NMM (161.6 mg, 1.6 mmol, 4.0 equiv). After being stirred at room temperature for 3 h, the resulting mixture was diluted with ethyl acetate (30 mL) and washed with water (2×30 mL) and brine (30 mL). The organic layer was concentrated under reduced pressure and telescoped to next step. ESI m/z=392.2 [M+Na]⁺. The resulting oil was dissolved in MeOH (4 mL) and then a solution of lithium hydroxide (LiOH) (28.8 mg, 1.2 mmol, 3.0 equiv) in water (2 mL) was added. After being stirred at room temperature for 2 h, the reaction mixture was acidified with hydrochloric acid (1N, 2 mL) and then extracted with ethyl acetate (3×20 mL). The organic layers was combined and concentrated to afford compound 6 as colorless oil. ESI m/z=378.4 [M+Na]⁺. The resulting oil was telescoped to next step without further purification.

Synthesis of (2S,4R)-4-(5-azidopentanamido)-N-((S)-1-(4-(4-methylthiazol-5-yl)phenyl)ethyl)pyrrolidine-2-carboxamide (7)

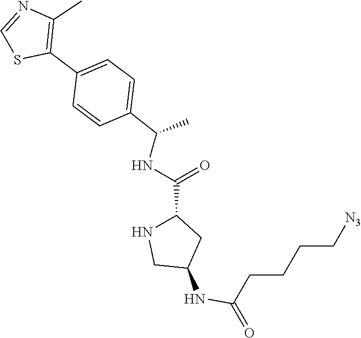 [SMILES: Cc1ncsc1-c1ccc([C@H](C)NC(=O)[C@@H]2C[C@@H](NC(=O)CCCCN=[N+]=[N-])CN2)cc1]

To a solution of the obtained compound 6 in DMSO (3 mL) were added (S)-1-(4-(4-methylthiazol-5-yl)phenyl)ethan-1-amine hydrochloride (101.9 mg, 0.4 mmol, 1.0 equiv), EDCI (153.4 mg, 0.8 mmol, 2.0 equiv), HOAt (108.8 mg, 0.8 mmol, 2.0 equiv) and NMM (161.6 mg, 1.6 mmol, 4.0 equiv). After being stirred at room temperature for 3 h, the resulting mixture was purified by preparative HPLC (10%-100% acetonitrile/0.1% TFA in H₂O) to afford colorless oil. ESI m/z=556.3 [M+H]⁺. The obtained oil was dissolved in dichloromethane (2 mL) and TFA (trifluoroacetic acid) (2 mL) was added at room temperature. After being stirred at room temperature for 30 min, the resulting mixture was concentrated and purified by preparative HPLC (10%-100% acetonitrile/0.1% TFA in H₂O) to afford the compound 7 as colorless oil (72.0 mg, 32% for four steps) ¹H NMR (600 MHz, Methanol-d₄) δ 9.08 (s, 1H), 7.40 (d, J=8.4 Hz, 2H), 7.37 (d, J=8.4 Hz, 2H), 5.03-4.97 (m, 1H), 4.50 (t, J=8.1 Hz, 1H), 4.36-4.30 (m, 1H), 3.56 (dd, J=12.1, 6.7 Hz, 1H), 3.26-3.19 (m, 3H), 2.43 (s, 3H), 2.38 (ddd, J=13.4, 8.4, 4.8 Hz, 1H), 2.23-2.13 (m, 3H), 1.67-1.56 (m, 2H), 1.55-1.48 (m, 2H), 1.44 (d, J=7.1 Hz, 3H). ¹³C NMR (151 MHz, Methanol-d₄) δ 174.59, 166.89, 152.49, 146.08, 144.22, 132.89, 129.54, 129.27, 126.35, 58.89, 50.71, 49.84, 49.24, 48.89, 35.20, 34.74, 27.99, 22.47, 20.87, 13.75. ESI m/z=456.4 [M+H]⁺.

Synthesis of (2S,4R)-1-((S)-2-amino-3,3-dimethylbutanoyl)-4-(5-azidopentanamido)-N-((S)-1-(4-(4-methylthiazol-5-yl)phenyl)ethyl)pyrrolidine-2-carboxamide (8)

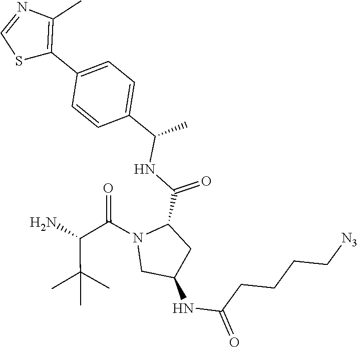 [SMILES: Cc1ncsc1-c1ccc([C@H](C)NC(=O)[C@@H]2C[C@@H](NC(=O)CCCCN=[N+]=[N-])CN2C(=O)[C@@H](N)C(C)(C)C)cc1]

To a solution of compound 7 (72.0 mg, 0.13 mmol, 1.0 equiv) in DMSO (2 mL) were added (S)-2-((tert-butoxycarbonyl)amino)-3,3-dimethylbutanoic acid (43.8 mg, 0.19 mmol, 1.5 equiv), EDCI (49.8 mg, 0.26 mmol, 2.0 equiv), HOAt (35.4 mg, 0.26 mmol, 2.0 equiv) and NMM (52.5 mg, 0.52 mmol, 4.0 equiv). After being stirred at room temperature for 2 h, the resulting mixture was purified by preparative HPLC (10%-100% acetonitrile/0.1% TFA in H₂O) to afford colorless oil. ESI m/z=669.5 [M+H]⁺. The obtained oil was dissolved in dichloromethane (2 mL) and TFA (trifluoroacetic acid) (2 mL) was added at room temperature. After being stirred at room temperature for 30 min, the resulting mixture was concentrated and purified by preparative HPLC (10%-100% acetonitrile/0.1% TFA in H₂O) to afford the compound 8 as white oil (72.8 mg, 82% for two steps). ¹H NMR (600 MHz, Methanol-d₄) δ 9.17 (s, 1H), 7.49 (d, J=8.0 Hz, 2H), 7.45 (d, J=8.1 Hz, 2H), 5.01 (q, J=7.5 Hz, 1H), 4.68 (t, J=7.6 Hz, 1H), 4.46 (p, J=5.2 Hz, 1H), 4.05 (s, 1H), 3.91 (dd, J=10.8, 5.9 Hz, 1H), 3.74 (dd, J=10.8, 3.9 Hz, 1H), 3.32 (t, J=6.7 Hz, 2H), 2.53 (s, 3H), 2.31 (ddd, J=13.3, 8.3, 5.0 Hz, 1H), 2.26 (t, J=7.4 Hz, 2H), 2.17-2.07 (m, 1H), 1.77-1.65 (m, 2H), 1.65-1.58 (m, 2H), 1.52 (d, J=7.0 Hz, 3H), 1.17 (s, 9H). ¹³C NMR (151 MHz, Methanol-d₄) δ 174.43, 170.91, 167.10, 152.41, 146.08, 144.71, 133.00, 129.31, 129.18, 126.33, 59.08, 58.91, 53.06, 50.73, 49.24, 48.82, 34.75, 34.55, 34.31, 28.00, 25.28, 22.58, 20.97, 13.72. ESI m/z=569.3 [M+H]⁺.

Synthesis of (2S,4R)-4-(5-azidopentanamido)-1-((S)-2-(tert-butyl)-15-((S)-4-(4-chlorophenyl)-2,3,9-trimethyl-6H-thieno[3,2-f][1,2,4]triazolo[4,3-a][1,4]diazepin-6-yl)-4,14-dioxo-6,10-dioxa-3,13-diazapentadecanoyl)-N-((S)-1-(4-(4-methylthiazol-5-yl)phenyl)ethyl)pyrrolidine-2-carboxamide (9)

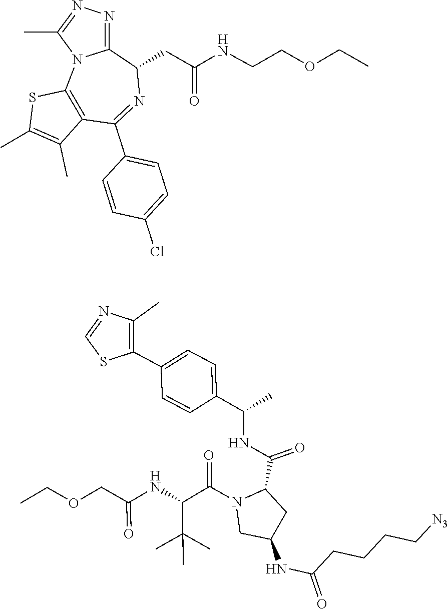

A similar synthesis, or synthesis of intermediates is described in Raina, K. Proc. Natl. Acad. Sci. 113 (2016): 7124-7129, which is hereby incorporated by reference in its entirety. Briefly, to a solution of compound 8 (25.0 mg, 0.044 mmol, 1.1 equiv) in DMSO (2 mL) were added (S)-2-(3-(2-(2-(4-(4-chlorophenyl)-2,3,9-trimethyl-6H-thieno[3,2-f][1,2,4]triazolo[4,3-a][1,4]diazepin-6-yl)acetamido)ethoxy)propoxy)acetic acid (22.4 mg, 0.040 mmol, 1.0 equiv), EDCI (15.3 mg, 0.08 mmol, 2.0 equiv), HOAt (10.9 mg, 0.08 mmol, 2.0 equiv) and NMM (16.2 mg, 0.16 mmol, 4.0 equiv). After being stirred at room temperature for 3 h, the resulting mixture was purified by preparative HPLC (10%-100% acetonitrile/0.1% TFA in H₂O) to afford the compound 9 as white solid (26.6 mg, 60%). ¹H NMR (600 MHz, Methanol-d₄) δ 9.07 (s, 1H), 7.42-7.30 (m, 8H), 4.89 (q, J=7.0 Hz, 1H), 4.61 (dd, J=8.8, 5.3 Hz, 1H), 4.51 (t, J=7.6 Hz, 1H), 4.48 (s, 1H), 4.34 (p, J=5.0 Hz, 1H), 3.92 (d, J=15.4 Hz, 1H), 3.86 (d, J=15.4 Hz, 1H), 3.81 (dd, J=10.9, 5.7 Hz, 1H), 3.74 (dd, J=10.9, 3.7 Hz, 1H), 3.60-3.45 (m, 6H), 3.42-3.33 (m, 3H), 3.29-3.23 (m, 1H), 3.17 (t, J=6.7 Hz, 2H), 2.65 (s, 3H), 2.40 (s, 3H), 2.36 (s, 3H), 2.17-2.06 (m, 3H), 2.03-1.96 (m, 1H), 1.83-1.77 (m, 2H), 1.60 (s, 3H), 1.58-1.51 (m, 2H), 1.50-1.43 (m, 2H), 1.38 (d, J=7.0 Hz, 3H), 0.95 (s, 9H). ¹³C NMR (151 MHz, Methanol-d₄) δ 174.29, 171.18, 170.80, 170.40, 165.26, 152.33, 146.14, 144.60, 137.02, 136.14, 132.56, 132.02, 130.91, 130.64, 130.23, 130.16, 129.35, 129.17, 129.14, 128.49, 126.37, 126.23, 69.44, 68.86, 68.56, 67.32, 58.75, 57.02, 53.49, 53.24, 50.70, 49.17, 48.76, 39.18, 35.15, 34.78, 34.59, 29.45, 27.97, 25.57, 22.82, 22.61, 20.97, 13.79, 13.04, 11.59, 10.17. ESI m/z=1110.2 [M+H]⁺.

Synthesis of N²-(4-(((2-amino-4-oxo-3,4-dihydropteridin-6-yl)methyl)amino)benzoyl)-N⁵-((1-(5-(((3R,5S)-1-((S)-2-(2-(3-(2-(2-((S)-4-(4-chlorophenyl)-2,3,9-trimethyl-6H-thieno[3,2-f][1,2,4]triazolo[4,3-a][1,4]diazepin-6-yl)acetamido)ethoxy)propoxy)acetamido)-3,3-dimethylbutanoyl)-5-(((S)-1-(4-(4-methylthiazol-5-yl)phenyl)ethyl)carbamoyl)pyrrolidin-3-yl)amino)-5-oxopentyl)-1H-1,2,3-triazol-4-yl)methyl)-L-glutamine (Folate-ARV-771N)

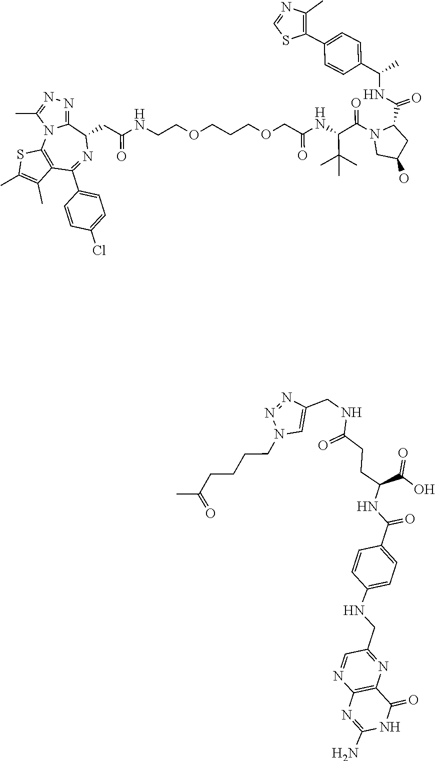 [SMILES: Cc1ncsc1-c1ccc([C@H](C)NC(=O)[C@@H]2C[C@@H](O)CN2C(=O)[C@@H](NC(=O)COCCCOCCNC(=O)C[C@@H]2N=C(c3ccc(Cl)cc3)c3c(sc(C)c3C)-n3c(C)nnc32)C(C)(C)C)cc1 ; CC(=O)CCCCn1cc(CNC(=O)CC[C@H](NC(=O)c2ccc(NCc3cnc4nc(N)[nH]c(=O)c4n3)cc2)C(=O)O)nn1]

To a solution of compound 9 (26.6 mg, 0.024 mmol, 1.0 equiv) in DMF (1.0 mL)/water (0.5 mL) were added compound 4 (13.8 mg, 0.029 mmol, 1.2 equiv), sodium ascorbate (1.9 mg, 0.01 mmol, 0.4 equiv) and CuSO₄.5H₂O (1.3 mg, 0.005 mmol, 0.2 equiv) at room temperature. The reaction mixture was heated to 50° C. After being stirred at 50° C. for 2 h, the resulting mixture was purified by preparative HPLC (10%-100% acetonitrile/0.1% TFA in H₂O) to afford Folate-ARV-771N as light yellow solid (15.8 mg, 41%). ¹H NMR (600 MHz, DMSO-d₆) δ 8.92 (s, 1H), 8.67 (s, 1H), 8.35 (d, J=7.6 Hz, 1H), 8.28 (t, J=5.7 Hz, 1H), 8.22 (t, J=5.7 Hz, 1H), 8.15 (d, J=7.5 Hz, 1H), 8.03 (d, J=6.9 Hz, 1H), 7.80 (s, 1H), 7.60 (d, J=8.2 Hz, 2H), 7.41 (d, J=8.3 Hz, 2H), 7.39-7.33 (m, 4H), 7.33-7.26 (m, 3H), 6.59 (d, J=8.4 Hz, 2H), 4.84 (p, J=6.9 Hz, 1H), 4.53-4.38 (m, 5H), 4.32-4.12 (m, 6H), 3.93-3.80 (m, 2H), 3.75-3.69 (m, 1H), 3.48 (t, J=6.4 Hz, 2H), 3.46-3.41 (m, 3H), 3.36 (t, J=5.9 Hz, 2H), 3.29-3.12 (m, 4H), 2.54 (s, 3H), 2.38 (s, 3H), 2.33 (s, 3H), 2.23-2.12 (m, 2H), 2.08-1.94 (m, 4H), 1.90-1.78 (m, 2H), 1.77-1.62 (m, 4H), 1.55 (s, 3H), 1.43-1.33 (m, 2H), 1.30 (d, J=6.9 Hz, 3H), 0.88 (s, 9H). ¹³C NMR (151 MHz, DMSO-d₆) δ 174.30, 172.25, 172.01, 170.53, 170.12, 169.54, 169.13, 166.78, 163.65, 159.85, 155.57, 153.11, 152.03, 151.05, 150.50, 150.08, 148.33, 148.11, 145.39, 145.11, 137.09, 135.81, 132.63, 131.68, 131.39, 130.66, 130.37, 130.15, 130.12, 129.47, 129.40, 129.34, 128.92, 128.42, 126.79, 123.08, 121.97, 111.75, 69.88, 69.37, 68.50, 67.49, 58.59, 56.22, 54.22, 53.29, 52.58, 49.45, 48.52, 48.30, 46.22, 39.09, 37.86, 35.87, 35.05, 34.85, 34.73, 32.27, 29.91, 29.76, 26.93, 26.66, 22.87, 22.52, 16.37, 14.49, 13.12, 11.73. HRMS (ESI-TOF) calcd for C₇₆H₉₁ClN₂₁O₁₂S₂⁺[M+H]⁺ 1588.6281, found 1588.6280. HPLC>98%, t_{R}=4.21 min.

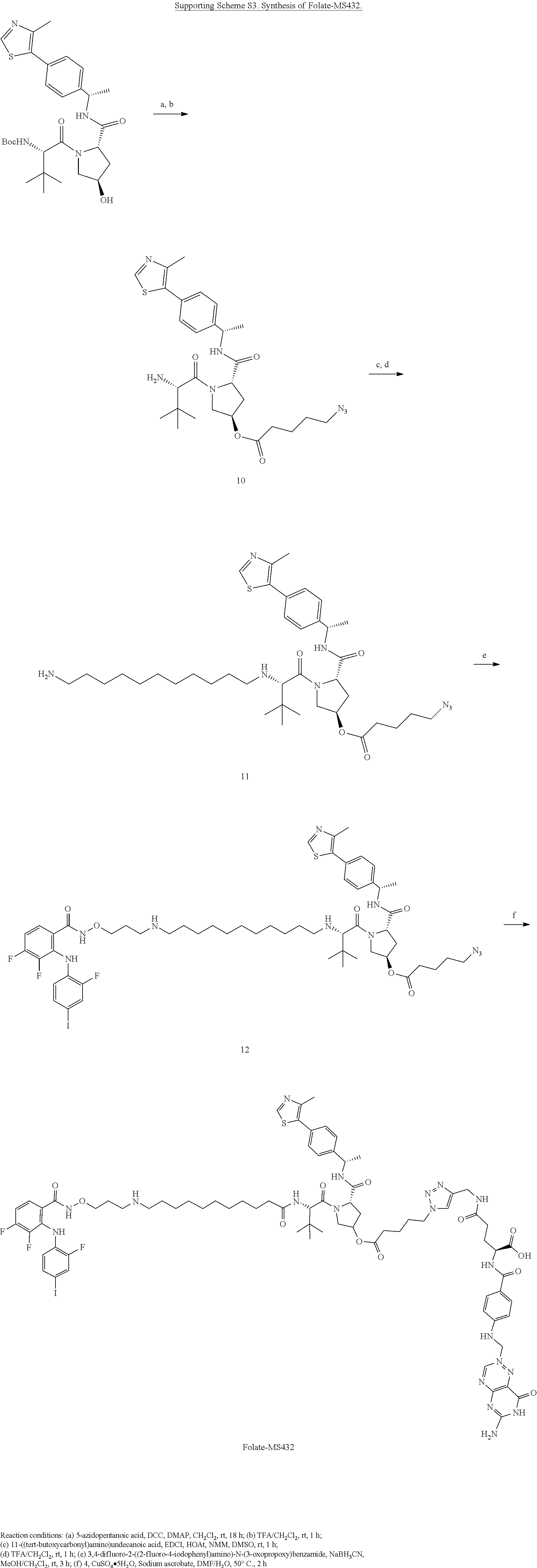

Synthesis of (3R,5S)-1-((S)-2-amino-3,3-dimethylbutanoyl)-5-(((S)-1-(4-(4-methylthiazol-5-yl)phenyl)ethyl)carbamoyl)pyrrolidin-3-yl 5-azidopentanoate (10)

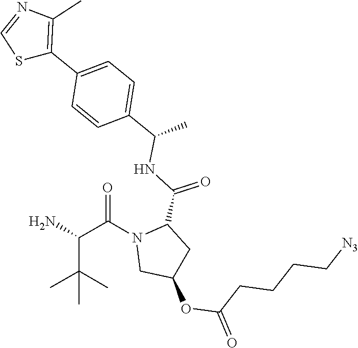

A similar synthesis, or synthesis of intermediates is described in Raina, K. Proc. Natl. Acad. Sci. 113 (2016): 7124-7129, which is hereby incorporated by reference in its entirety. Briefly, to a solution of tert-butyl ((S)-1-42S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamate (247 mg, 0.454 mmol, 1.0 equiv) in dichloromethane (5 mL) were added 5-azidopentanoic acid (97.4 mg, 0.68 mmol, 1.5 equiv), DCC (N,N′-Dicyclohexylcarbodiimide) (140.1 mg, 0.68 mmol, 1.5 equiv) and DMAP (4-Dimethylaminopyridine) (5.5 mg, 0.045 mmol, 0.1 equiv. After being stirred at room temperature for 18 h, the resulting mixture was purified by preparative HPLC (10%-100% acetonitrile/0.1% TFA in H₂O) to afford intermediate compound as white solid. ESI m/z=692.4 [M+Na]⁺. Dissolved the white solid in CH₂Cl₂ (2 mL) and then TFA (2 mL) was added to the reaction at room temperature. After being stirred at room temperature for 1 h, the resulting mixture was concentrated and purified by preparative HPLC (10%-100% acetonitrile/0.1% TFA in H₂O) to afford compound 10 as white solid in TFA salt form (238 mg, 77% for two steps). ¹H NMR (600 MHz, Methanol-d₄) δ 9.00 (s, 1H), 7.36 (d, J=8.3 Hz, 2H), 7.33 (d, J=8.3 Hz, 2H), 5.27 (t, J=4.3 Hz, 1H), 4.90 (q, J=6.9 Hz, 1H), 4.56 (dd, J=9.4, 7.7 Hz, 1H), 3.97 (s, 1H), 3.87 (d, J=12.1 Hz, 1H), 3.75 (dd, J=12.0, 4.1 Hz, 1H), 3.22 (t, J=6.6 Hz, 2H), 2.40 (s, 3H), 2.35-2.29 (m, 1H), 2.27 (t, J=7.3 Hz, 2H), 2.01 (ddd, J=14.1, 9.5, 4.7 Hz, 1H), 1.64-1.55 (m, 2H), 1.55-1.48 (m, 2H), 1.41 (d, J=7.0 Hz, 3H), 1.04 (s, 9H). ¹³C NMR (151 MHz, Methanol-d₄) δ 172.69, 170.76, 167.06, 152.24, 146.37, 144.56, 132.78, 129.48, 129.17, 126.31, 73.11, 59.37, 58.99, 54.03, 50.71, 48.87, 34.83, 34.44, 32.93, 27.85, 25.25, 21.64, 20.99, 13.84. ESI m/z=570.3 [M+H]⁺.

Synthesis of (3R,5S)-1-((S)-2-(11-aminoundecanamido)-3,3-dimethylbutanoyl)-5-(((S)-1-(4-(4-methylthiazol-5-yl)phenyl)ethyl)carbamoyl)pyrrolidin-3-yl 5-azidopentanoate (11)

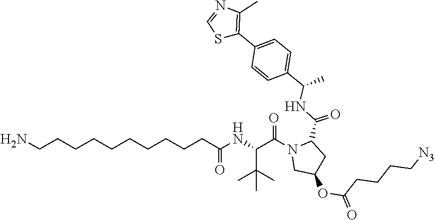

To a solution of compound 10 (50.0 mg, 0.073 mmol, 1.0 equiv) in DMSO (2 mL) were added 11-((tert-butoxycarbonyl)amino)undecanoic acid (26.4 mg, 0.088 mmol, 1.2 equiv), EDCI (28.0 mg, 0.146 mmol, 2.0 equiv), HOAt (19.9 mg, 0.146 mmol, 2.0 equiv) and NMM (29.3 mg, 0.29 mmol, 4.0 equiv). After being stirred at room temperature for 1 h, the resulting mixture was purified by preparative HPLC (10%-100% acetonitrile/0.1% TFA in H₂O) to afford colorless oil. ESI m/z=753.4 [M−Boc+2H]⁺. The obtained oil was dissolved in dichloromethane (2 mL) and TFA (1 mL) was added at room temperature. After being stirred at room temperature for 1 h, the resulting mixture was concentrated and purified by preparative HPLC (10%-100% acetonitrile/0.1% TFA in H₂O) to afford the compound 11 as white solid (34.0 mg, 54% for two steps). ¹H NMR (600 MHz, Methanol-d₄) δ 9.23 (s, 1H), 7.49 (d, J=8.2 Hz, 2H), 7.46 (d, J=8.2 Hz, 2H), 5.34 (d, J=4.4 Hz, 1H), 5.09-5.02 (m, 1H), 4.59 (t, J=8.5 Hz, 1H), 4.52 (s, 1H), 4.18 (d, J=11.8 Hz, 1H), 3.87 (dd, J=11.8, 4.0 Hz, 1H), 3.33 (t, J=6.6 Hz, 2H), 2.93 (t, J=7.7 Hz, 2H), 2.53 (s, 3H), 2.46-2.21 (m, 5H), 2.13 (ddd, J=13.9, 9.3, 4.8 Hz, 1H), 1.75-1.57 (m, 6H), 1.53 (d, J=7.0 Hz, 3H), 1.45-1.30 (m, 14H), 1.07 (s, 9H). ¹³C NMR (151 MHz, Methanol-d₄) δ 174.76, 172.87, 171.28, 171.07, 152.57, 145.74, 144.82, 133.26, 129.27, 129.16, 126.41, 73.14, 59.02, 57.90, 53.97, 50.73, 48.82, 39.36, 35.14, 34.64, 34.62, 32.99, 29.11, 29.07, 29.05, 28.96, 28.81, 27.90, 27.19, 26.06, 25.67, 25.64, 21.61, 20.98, 13.60. ESI m/z=753.6 [M+H]⁺.

Synthesis of (3R,5S)-1-((S)-20-(tert-butyl)-1-(3,4-difluoro-2-((2-fluoro-4-iodophenyl)amino)phenyl)-1,18-dioxo-3-oxa-2,7,19-triazahenicosan-21-oyl)-5-(((S)-1-(4-(4-methylthiazol-5-yl)phenyl)ethyl)carbamoyl)pyrrolidin-3-yl 5-azidopentanoate (12)

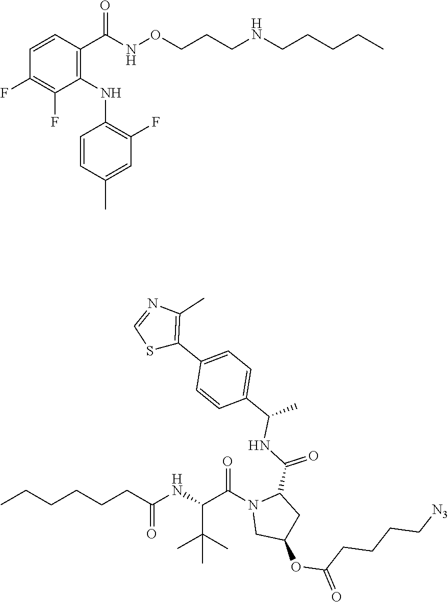

A similar synthesis, or synthesis of intermediates used herein is described in Wei, J. J. Med. Chem. 62 (2019): 10897-10911, which is hereby incorporated by reference in its entirety. Briefly, to a solution of compound 11 (34.0 mg, 0.04 mmol, 1.0 equiv) in MeOH/CH₂Cl₂ (½ mL) were added 3,4-difluoro-2-((2-fluoro-4-iodophenyl)amino)-N-(3-oxopropoxy)benzamide (18.6 mg, 0.04 mmol, 1.0 equiv) and NaBH₃CN (3.8 mg, 0.06 mmol, 1.5 equiv). After being stirred at room temperature for 3 h, the resulting mixture was purified by preparative HPLC (10%-100% acetonitrile/0.1% TFA in H₂O) to afford compound 12 as light yellow solid (18.1 mg, 34%). ¹H NMR (600 MHz, Methanol-d₄) δ 8.98 (s, 1H), 7.44-7.20 (m, 7H), 6.95 (td, J=9.2, 6.9 Hz, 1H), 6.54 (td, J=8.8, 4.4 Hz, 1H), 5.25-5.18 (m, 1H), 4.91 (q, J=7.1 Hz, 1H), 4.50-4.44 (m, 1H), 4.40 (s, 1H), 4.06 (d, J=11.5 Hz, 1H), 4.00-3.95 (m, 2H), 3.75 (dd, J=11.8, 4.0 Hz, 1H), 3.19 (t, J=6.6 Hz, 2H), 3.14 (t, J=5.9 Hz, 2H), 2.94 (dd, J=9.0, 6.7 Hz, 2H), 2.40 (s, 3H), 2.32-2.22 (m, 3H), 2.22-2.10 (m, 2H), 2.01 (ddd, J=13.9, 9.3, 4.8 Hz, 1H), 1.97-1.90 (m, 2H), 1.67-1.54 (m, 4H), 1.53-1.46 (m, 4H), 1.41 (d, J=7.0 Hz, 3H), 1.34-1.26 (m, 2H), 1.26-1.14 (m, 10H), 0.94 (s, 9H). ¹³C NMR (151 MHz, Methanol-d₄) δ 174.71, 172.86, 171.26, 171.08, 153.12 (d, J=247.6 Hz), 152.15, 144.55, 133.26 (d, J=4.5 Hz), 131.10 (d, J=10.6 Hz), 129.54, 129.15, 126.33, 126.18, 124.40, 123.93 (d, J=21.1 Hz), 119.68, 118.69, 110.22 (d, J=18.1 Hz), 81.18(d, J=7.6 Hz), 76.36, 73.15, 59.03, 57.89, 53.98, 50.73, 48.81, 48.02, 47.23, 35.16, 34.63, 33.00, 29.18, 29.10, 29.01, 28.84, 27.91, 26.23, 26.04, 25.68, 25.65, 25.60, 24.33, 21.61, 20.97, 13.92. ESI m/z=1201.8 [M+H]⁺.

Synthesis of N²-(4-(((2-amino-4-oxo-3,4-dihydropteridin-6-yl)methyl)amino)benzoyl)-N⁵-((1-(5-(((3R,5S)-1-((S)-2-(11-((3-((3,4-difluoro-2-((2-fluoro-4-iodophenyl)amino)benzamido)oxy)propyl)amino)undecanamido)-3,3-dimethylbutanoyl)-5-(((S)-1-(4-(4-methylthiazol-5-yl)phenyl)ethyl)carbamoyl)pyrrolidin-3-yl)oxy)-5-oxopentyl)-1H-1,2,3-triazol-4-yl)methyl)-L-glutamine (Folate-MS432)

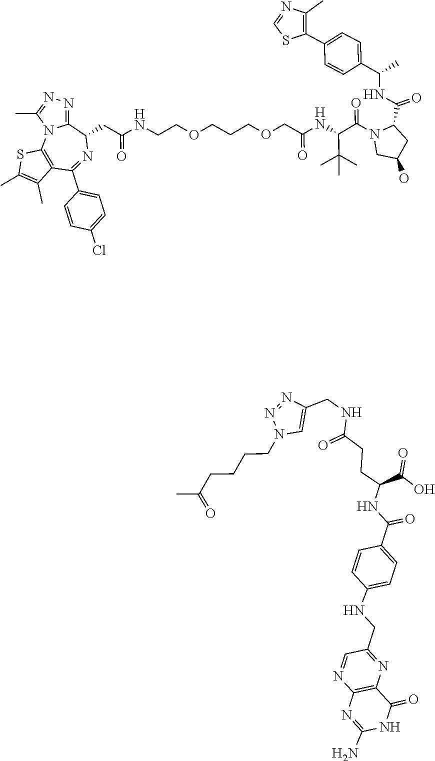 [SMILES: Cc1ncsc1-c1ccc([C@H](C)NC(=O)[C@@H]2C[C@@H](O)CN2C(=O)[C@@H](NC(=O)COCCCOCCNC(=O)C[C@@H]2N=C(c3ccc(Cl)cc3)c3c(sc(C)c3C)-n3c(C)nnc32)C(C)(C)C)cc1 ; CC(=O)CCCCn1cc(CNC(=O)CC[C@H](NC(=O)c2ccc(NCc3cnc4nc(N)[nH]c(=O)c4n3)cc2)C(=O)O)nn1]

To a solution of compound 12 (18.1 mg, 0.014 mmol, 1.0 equiv) in DMF (1.0 mL)/water (0.5 mL) were added compound 4 (10.0 mg, 0.021 mmol, 1.5 equiv), sodium ascorbate (1.1 mg, 0.006 mmol, 0.4 equiv) and CuSO₄·5H₂O (0.7 mg, 0.003 mmol, 0.2 equiv) at room temperature. The reaction mixture was heated to 50° C. After being stirred at 50° C. for 2 h, the resulting mixture was purified by preparative HPLC (10%-100% acetonitrile/0.1% TFA in H₂O) to afford Folate-MS432 as light yellow solid (17.0 mg, 68%). ¹H NMR (600 MHz, DMSO-d₆) δ 12.11 (s, 1H), 8.91 (s, 1H), 8.70-8.58 (m, 2H), 8.48 (s, 2H), 8.36 (d, J=7.7 Hz, 1H), 8.28 (t, J=5.7 Hz, 1H), 8.14 (d, J=7.6 Hz, 1H), 7.80 (s, 1H), 7.74 (d, J=8.7 Hz, 1H), 7.59 (d, J=8.5 Hz, 2H), 7.54-7.46 (m, 1H), 7.36 (d, J=8.2 Hz, 2H), 7.31 (d, J=8.3 Hz, 2H), 7.21-7.11 (m, 1H), 6.68-6.53 (m, 3H), 5.14 (s, 1H), 4.85 (p, J=7.1 Hz, 1H), 4.45 (s, 2H), 4.39 (t, J=8.4 Hz, 1H), 4.32-4.15 (m, 4H), 3.97-3.84 (m, 3H), 3.67 (dd, J=11.7, 4.0 Hz, 1H), 3.07-2.96 (m, 2H), 2.89-2.77 (m, 2H), 2.38 (s, 3H), 2.31-2.11 (m, 6H), 2.07-1.97 (m, 2H), 1.97-1.89 (m, 1H), 1.88-1.81 (m, 3H), 1.75-1.69 (m, 2H), 1.54-1.35 (m, 6H), 1.31 (d, J=7.0 Hz, 3H), 1.26-1.06 (m, 14H), 0.88 (s, 9H). ¹³C NMR (151 MHz, DMSO-d₆) δ 174.30, 172.98, 172.71, 172.01, 170.41, 170.32, 166.83, 165.37, 160.86, 153.91, 153.85, 152.21, 151.97, 151.17, 150.24, 148.75, 148.22, 145.45, 145.01, 133.69, 132.17, 131.61, 130.21, 129.46, 129.32, 128.42, 126.83, 125.26, 124.17 (d, J=21.1 Hz), 123.05, 121.86, 121.02, 120.58, 111.69, 110.95 (d, J=18.1 Hz), 82.57 (d, J=6.0 Hz), 74.32, 73.53, 58.68, 57.49, 54.00, 52.55, 49.33, 48.30, 47.46, 46.32, 45.50, 40.86, 35.19, 34.96, 34.74, 33.19, 32.26, 29.49, 29.37, 29.29, 29.24, 29.12, 28.98, 26.95, 26.86, 26.40, 26.02, 25.90, 24.83, 22.88, 21.62, 16.42. HRMS (ESI-TOF) calcd for C₇₇H₉₅F₃IN₁₈O₁₂S⁺[M+H]⁺ 1679.6089, found 1679.6082. HPLC>97%, t_{R}=4.59 min.

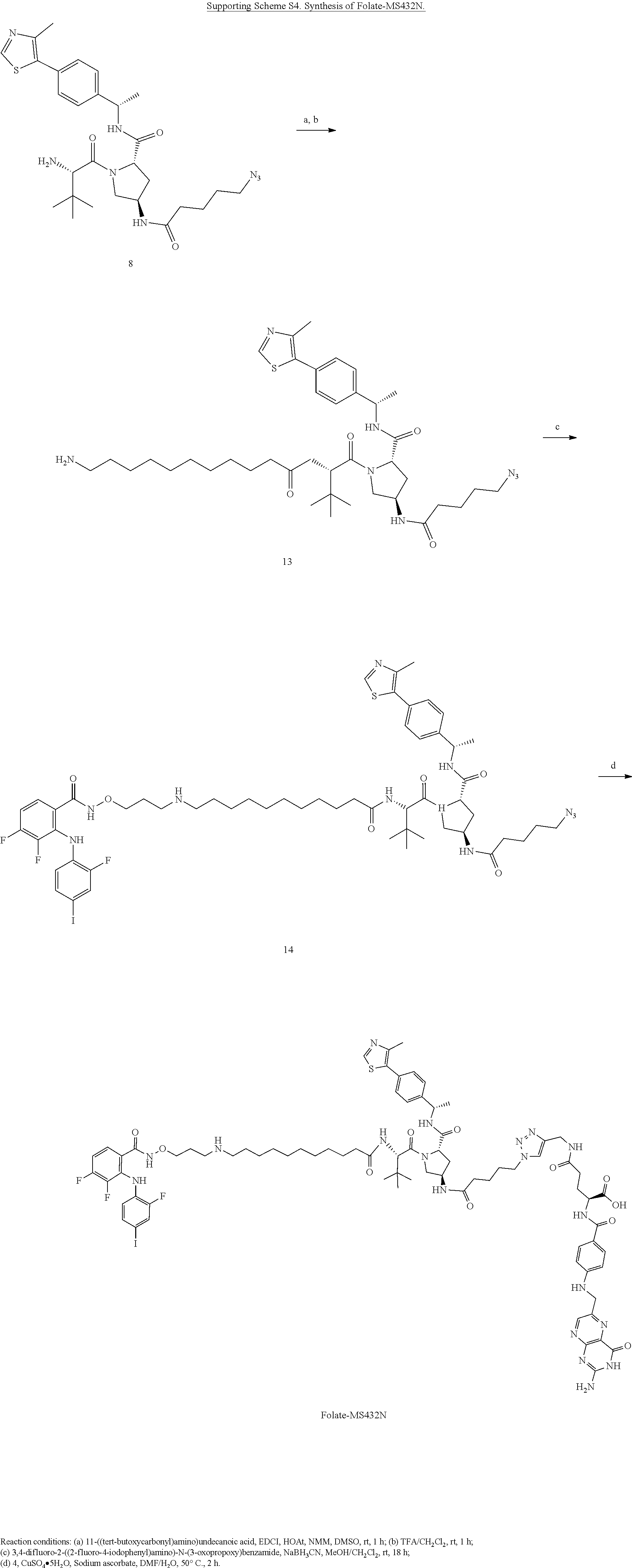

Synthesis of (2S,4R)-1-((S)-2-(11-aminoundecanamido)-3,3-dimethylbutanoyl)-4-(5-azidopentanamido)-N-((S)-1-(4-(4-methylthiazol-5-yl)phenyl)ethyl)pyrrolidine-2-carboxamide (13)

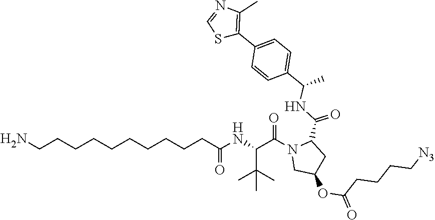

To a solution of compound 8 (34.1 mg, 0.05 mmol, 1.0 equiv) in DMSO (1 mL) were added 11-((tert-butoxycarbonyl)amino)undecanoic acid (30.1 mg, 0.10 mmol, 2.0 equiv), EDCI (19.2 mg, 0.10 mmol, 2.0 equiv), HOAt (13.6 mg, 0.10 mmol, 2.0 equiv) and NMM (20.2 mg, 0.20 mmol, 4.0 equiv). After being stirred at room temperature for 1 h, the resulting mixture was purified by preparative HPLC (10%-100% acetonitrile/0.1% TFA in H₂O) to afford colorless oil. ESI m/z=852.5 [M+H]⁺. The obtained oil was dissolved in dichloromethane (2 mL) and TFA (1 mL) was added at room temperature. After being stirred at room temperature for 1 h, the resulting mixture was concentrated and purified by preparative HPLC (10%400% acetonitrile/0.1% TFA in H₂O) to afford the compound 13 as white solid (23.0 mg, 53% for two steps). ¹H NMR (600 MHz, Methanol-d₄) δ 9.00 (s, 1H), 7.48 (d, J=8.4 Hz, 2H), 7.44 (d, J=8.3 Hz, 2H), 5.02 (q, J=6.9 Hz, 1H), 4.59 (t, J=7.7 Hz, 1H), 4.49-4.40 (m, 2H), 4.00-3.92 (m, 1H), 3.88 (dd, J=10.9, 5.4 Hz, 1H), 3.37-3.32 (m, 2H), 2.93 (t, J=7.7 Hz, 2H), 2.51 (s, 3H), 2.39-2.21 (m, 5H), 2.14-2.06 (m, 1H), 1.77-1.57 (m, 6H), 1.52 (d, J=7.0 Hz, 3H), 1.45-1.31 (m, 14H), 1.09 (s, 9H). ¹³C NMR (151 MHz, Methanol-d₄) δ 175.12, 174.30, 171.37, 171.23, 152.63, 145.73, 144.84, 133.30, 129.18, 129.10, 126.40, 58.51, 53.15, 50.71, 49.27, 48.77, 39.37, 34.89, 34.86, 34.58, 34.16, 29.13, 29.10, 28.97, 28.83, 28.02, 27.20, 26.08, 25.66, 25.54, 22.64, 20.99, 13.58. ESI m/z=752.6 [M+H]⁺.

Synthesis of (2S,4R)-4-(5-azidopentanamido)-1-((S)-20-(tert-butyl)-1-(3,4-difluoro-2-((2-fluoro-4-iodophenyl)amino)phenyl)-1,18-dioxo-3-oxa-2,7,19-triazahenicosan-21-oyl)-N-((S)-1-(4-(4-methylthiazol-5-yl)phenyl)ethyl)pyrrolidine-2-carboxamide (14)

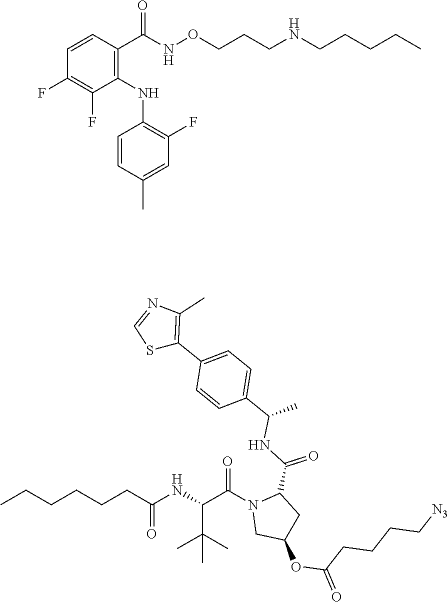

A similar synthesis, or synthesis of intermediates used herein is described in Wei, J. J. Med. Chem. 62 (2019): 10897-10911, which is hereby incorporated by reference in its entirety. Briefly, to a solution of compound 13 (17.3 mg, 0.02 mmol, 1.0 equiv) in MeOH/CH₂Cl₂ (0.5/1.0 mL) were added 3,4-difluoro-2-((2-fluoro-4-iodophenyl)amino)-N-(3-oxopropoxy)benzamide (9.2 mg, 0.02 mmol, 1.0 equiv) and NaBH₃CN (2.5 mg, 0.04 mmol, 2.0 equiv). After being stirred at room temperature for 18 h, the resulting mixture was purified by preparative HPLC (10%-100% acetonitrile/0.1% TFA in H₂O) to afford compound 14 as light yellow solid (5.0 mg, 19%). ¹H NMR (600 MHz, Methanol-d₄) δ 8.97 (s, 1H), 7.54-7.36 (m, 7H), 7.13-7.05 (m, 1H), 6.66 (td, J=8.8, 4.4 Hz, 1H), 5.02 (q, J=6.8 Hz, 1H), 4.59 (t, J=7.7 Hz, 1H), 4.46-4.41 (m, 2H), 4.13-4.07 (m, 2H), 3.96 (dd, J=10.7, 3.5 Hz, 1H), 3.87 (dd, J=10.9, 5.5 Hz, 1H), 3.31 (t, J=6.7 Hz, 2H), 3.26 (t, J=5.8 Hz, 2H), 3.10-3.03 (m, 2H), 2.51 (s, 3H), 2.38-2.22 (m, 5H), 2.16-2.08 (m, 1H), 2.07-2.02 (m, 2H), 1.79-1.56 (m, 8H), 1.52 (d, J=7.0 Hz, 3H), 1.45-1.28 (m, 12H), 1.08 (s, 9H). ESI m/z=1200.2 [M+H]⁺.

Synthesis of N²-(4-(((2-amino-4-oxo-3,4-dihydropteridin-6-yl)methyl)amino)benzoyl)-N⁵-((1-(5-(((3R,5S)-1-((S)-2-(11-((3-((3,4-difluoro-2-((2-fluoro-4-iodophenyl)amino)benzamido)oxy)propyl)amino)undecanamido)-3,3-dimethylbutanoyl)-5-(((S)-1-(4-(4-methylthiazol-5-yl)phenyl)ethyl)carbamoyl)pyrrolidin-3-yl)amino)-5-oxopentyl)-1H-1,2,3-triazol-4-yl)methyl)-L-glutamine (Folate-MS432N)

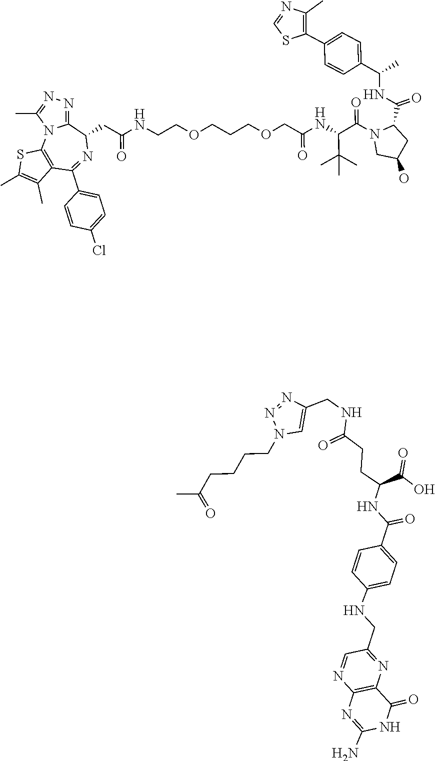 [SMILES: Cc1ncsc1-c1ccc([C@H](C)NC(=O)[C@@H]2C[C@@H](O)CN2C(=O)[C@@H](NC(=O)COCCCOCCNC(=O)C[C@@H]2N=C(c3ccc(Cl)cc3)c3c(sc(C)c3C)-n3c(C)nnc32)C(C)(C)C)cc1 ; CC(=O)CCCCn1cc(CNC(=O)CC[C@H](NC(=O)c2ccc(NCc3cnc4nc(N)[nH]c(=O)c4n3)cc2)C(=O)O)nn1]

To a solution of compound 14 (5.0 mg, 0.004 mmol, 1.0 equiv) in DMF (0.8 mL)/water (0.4 mL) were added compound 4 (2.8 mg, 0.006 mmol, 1.5 equiv), sodium ascorbate (1.2 mg, 0.006 mmol, 1.5 equiv) and CuSO₄·5H₂O (1.0 mg, 0.004 mmol, 1.0 equiv) at room temperature. The reaction mixture was heated to 50° C. After being stirred at 50° C. for 2 h, the resulting mixture was purified by preparative HPLC (10%-100% acetonitrile/0.1% TFA in H₂O) to afford Folate-MS432N as light yellow solid (3.6 mg, 50%). ¹H NMR (600 MHz, DMSO-d₆) δ 12.14 (s, 1H), 8.99 (s, 1H), 8.66 (s, 1H), 8.46-8.37 (m, 2H), 8.35-8.29 (m, 2H), 8.20 (d, J=7.6 Hz, 1H), 8.03 (d, J=6.7 Hz, 1H), 7.89-7.81 (m, 2H), 7.73-7.63 (m, 2H), 7.58 (dd, J=10.8, 2.0 Hz, 1H), 7.49-7.33 (m, 6H), 7.25 (q, J=8.7 Hz, 1H), 6.71-6.61 (m, 3H), 4.91 (p, J=7.2 Hz, 1H), 4.56-4.46 (m, 3H), 4.40 (d, J=6.0 Hz, 1H), 4.33-4.22 (m, 5H), 3.93 (t, J=5.7 Hz, 2H), 3.81-3.73 (m, 1H), 3.12-3.04 (m, 2H), 2.94-2.86 (m, 2H), 2.45 (s, 3H), 2.31-2.19 (m, 3H), 2.15-1.86 (m, 9H), 1.80-1.72 (m, 2H), 1.61-1.40 (m, 6H), 1.37 (d, J=7.0 Hz, 3H), 1.22 (d, J=8.8 Hz, 14H), 0.96 (s, 9H). HRMS (ESI-TOF) calcd for C₇₇H₉₆F₃IN₁₉O₁₁S⁺[M+H]⁺ 1678.6249, found 1678.6233. HPLC>97%, t_{R}=4.61 min.

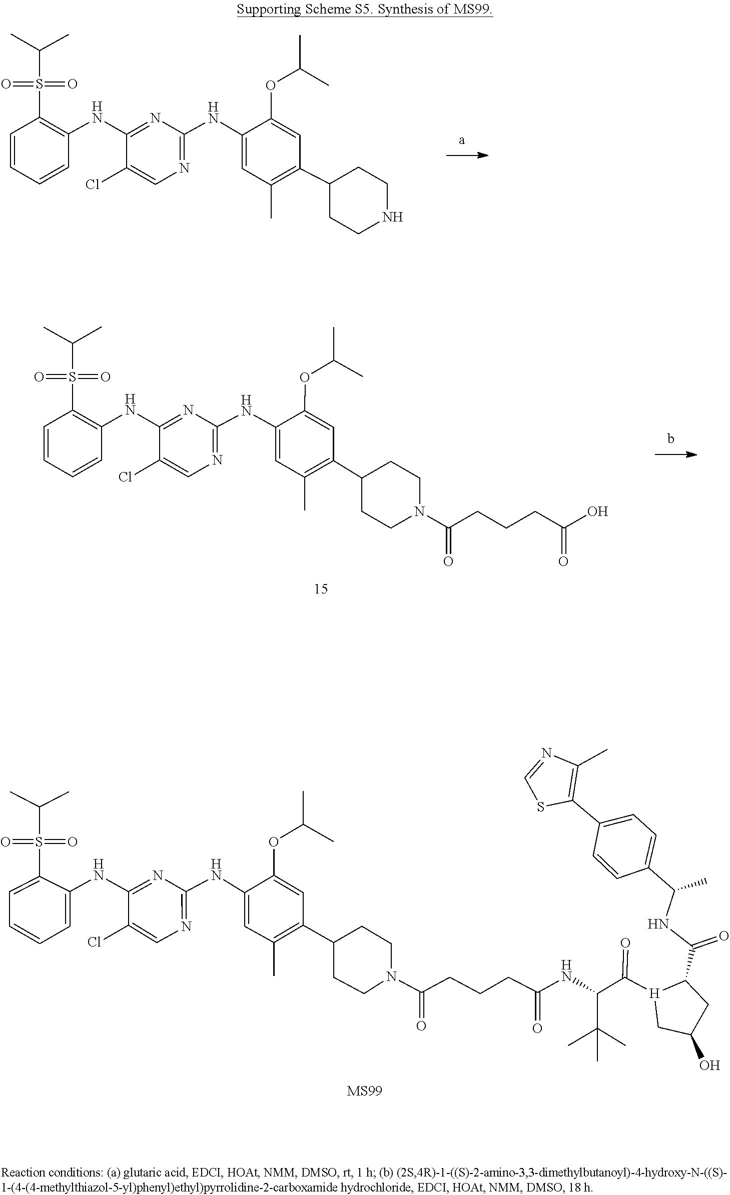

Synthesis of 5-(4-(4-((5-chloro-4-((2-(isopropylsulfonyl)phenyl)amino)pyrimidin-2-yl)amino)-5-isopropoxy-2-methylphenyl)piperidin-1-yl)-5-oxopentanoic acid (15)

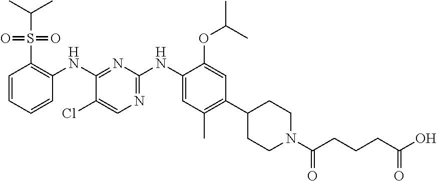 [SMILES: Cc1cc(Nc2ncc(Cl)c(Nc3ccccc3S(=O)(=O)C(C)C)n2)c(OC(C)C)cc1C1CCN(C(=O)CCCC(=O)O)CC1]

To a solution of 5-chloro-N²-(2-isopropoxy-5-methyl-4-(piperidin-4-yl)phenyl)-N⁴-(2-(isopropylsulfonyl)phenyl)pyrimidine-2,4-diamine (Ceritinib) (111.6 mg, 0.2 mmol, 1.0 equiv) in DMSO (3 mL) were added glutaric acid (132.0 mg, 1.0 mmol, 5.0 equiv), EDCI (76.7 mg, 0.4 mmol, 2.0 equiv), HOAt (54.4 mg, 0.4 mmol, 2.0 equiv) and NMM (202.0 mg, 2.0 mmol, 10 equiv). After being stirred at room temperature for 1 h, the resulting mixture was purified by preparative HPLC (10%-100% acetonitrile/0.1% TFA in H₂O) to afford compound 15 as white solid (123.0 mg, 91%). ¹H NMR (600 MHz, Methanol-d₄) δ 8.36 (d, J=8.1 Hz, 1H), 8.21 (s, 1H), 7.98 (dd, J=7.9, 1.6 Hz, 1H), 7.81-7.65 (m, 1H), 7.54-7.46 (m, 1H), 7.45 (s, 1H), 6.90 (s, 1H), 4.77-4.70 (m, 1H), 4.68-4.59 (m, 1H), 4.20-4.10 (m, 1H), 3.45-3.34 (m, 1H), 3.25 (td, J=13.2, 2.6 Hz, 1H), 3.05 (tt, J=12.1, 3.5 Hz, 1H), 2.75 (td, J=13.0, 2.7 Hz, 1H), 2.60-2.47 (m, 2H), 2.42 (t, J=7.2 Hz, 2H), 2.22 (s, 3H), 1.94 (p, J=7.3 Hz, 2H), 1.89-1.77 (m, 2H), 1.69 (qd, J=12.6, 4.1 Hz, 1H), 1.59 (qd, J=12.7, 4.2 Hz, 1H), 1.30-1.25 (m, 12H). ¹³C NMR (151 MHz, Methanol-d₄) δ 175.48, 171.82, 160.85, 157.25, 153.60, 148.12, 145.42, 141.60, 136.48, 134.84, 131.29, 127.33, 127.12, 125.58, 125.20, 124.02, 111.68, 105.56, 71.25, 55.61, 46.26, 42.34, 38.20, 32.79, 32.72, 32.09, 31.90, 21.00, 20.51, 17.61, 14.13. ESI m/z=672.4 [M+H]⁺.

Synthesis of (2S,4R)-1-((S)-2-(5-(4-(4-((5-chloro-4-((2-(isopropylsulfonyl)phenyl)amino)pyrimidin-2-yl)amino)-5-isopropoxy-2-methylphenyl)piperidin-1-yl)-5-oxopentanamido)-3,3-dimethylbutanoyl)-4-hydroxy-N-((S)-1-(4-(4-methylthiazol-5-yl)phenyl)ethyl)pyrrolidine-2-carboxamide (MS99)

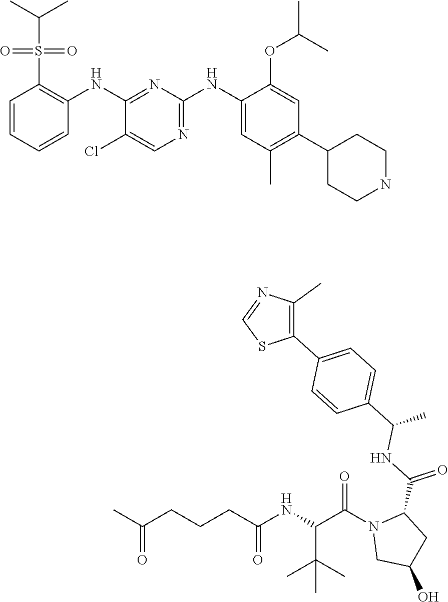

To a solution of compound 15 (25.0 mg, 0.037 mmol, 1.0 equiv) in DMSO (2 mL) were added (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide hydrochloride (132.0 mg, 0.042 mmol, 1.1 equiv), EDCI (14.2 mg, 0.074 mmol, 2.0 equiv), HOAt (10.1 mg, 0.074 mmol, 2.0 equiv) and NMM (15.0 mg, 0.149 mmol, 4 equiv). After being stirred at room temperature for 18 h, the resulting mixture was purified by preparative HPLC (10%-100% acetonitrile/0.1% TFA in H₂O) to afford MS99 as white solid (25.6 mg, 63%). ¹H NMR (600 MHz, Methanol-d₄) δ 9.02 (s, 1H), 8.32 (s, 1H), 8.23 (s, 1H), 8.01 (dd, J=8.0, 1.6 Hz, 1H), 7.75-7.70 (m, 1H), 7.54 (t, J=7.7 Hz, 1H), 7.46 (d, J=8.2 Hz, 2H), 7.43 (d, J=8.2 Hz, 2H), 7.41-7.31 (m, 1H), 6.92 (s, 1H), 5.06-4.98 (m, 1H), 4.73 (d, J=13.0 Hz, 1H), 4.68-4.62 (m, 2H), 4.60-4.56 (m, 1H), 4.45 (s, 1H), 4.13 (d, J=14.1 Hz, 1H), 3.91 (d, J=11.0 Hz, 1H), 3.77 (dd, J=11.0, 4.0 Hz, 1H), 3.40 (p, J=6.8 Hz, 1H), 3.25 (t, J=12.6 Hz, 1H), 3.06 (t, J=12.1 Hz, 1H), 2.76 (t, J=12.7 Hz, 1H), 2.50 (s, 3H), 2.44-2.34 (m, 3H), 2.27-2.12 (m, 4H), 2.06-1.92 (m, 3H), 1.87 (d, J=12.9 Hz, 1H), 1.82 (d, J=13.2 Hz, 1H), 1.75-1.64 (m, 1H), 1.63-1.57 (m, 2H), 1.56-1.47 (m, 3H), 1.33-1.24 (m, 12H), 1.08 (s, 9H). ¹³C NMR (151 MHz, Methanol-d₄) δ 173.96, 171.84, 171.15, 170.93, 157.67, 152.98, 151.98, 148.66, 146.83, 145.03, 144.56, 143.76, 142.29, 136.22, 134.83, 132.57, 131.36, 129.64, 129.10, 129.01, 127.79, 127.52, 126.31, 126.03, 125.67, 111.84, 105.63, 71.24, 69.58, 59.18, 57.77, 56.58, 55.60, 48.74, 48.47, 46.25, 42.28, 38.26, 37.39, 35.03, 34.49, 32.80, 32.06, 32.00, 25.71, 21.45, 20.99, 20.93, 17.50, 14.07. HRMS (ESI-TOF) calcd for C₅₆H₇₃ClN₉O₈S₂⁺[M+H]⁺ 1098.4707, found 1098.4723.

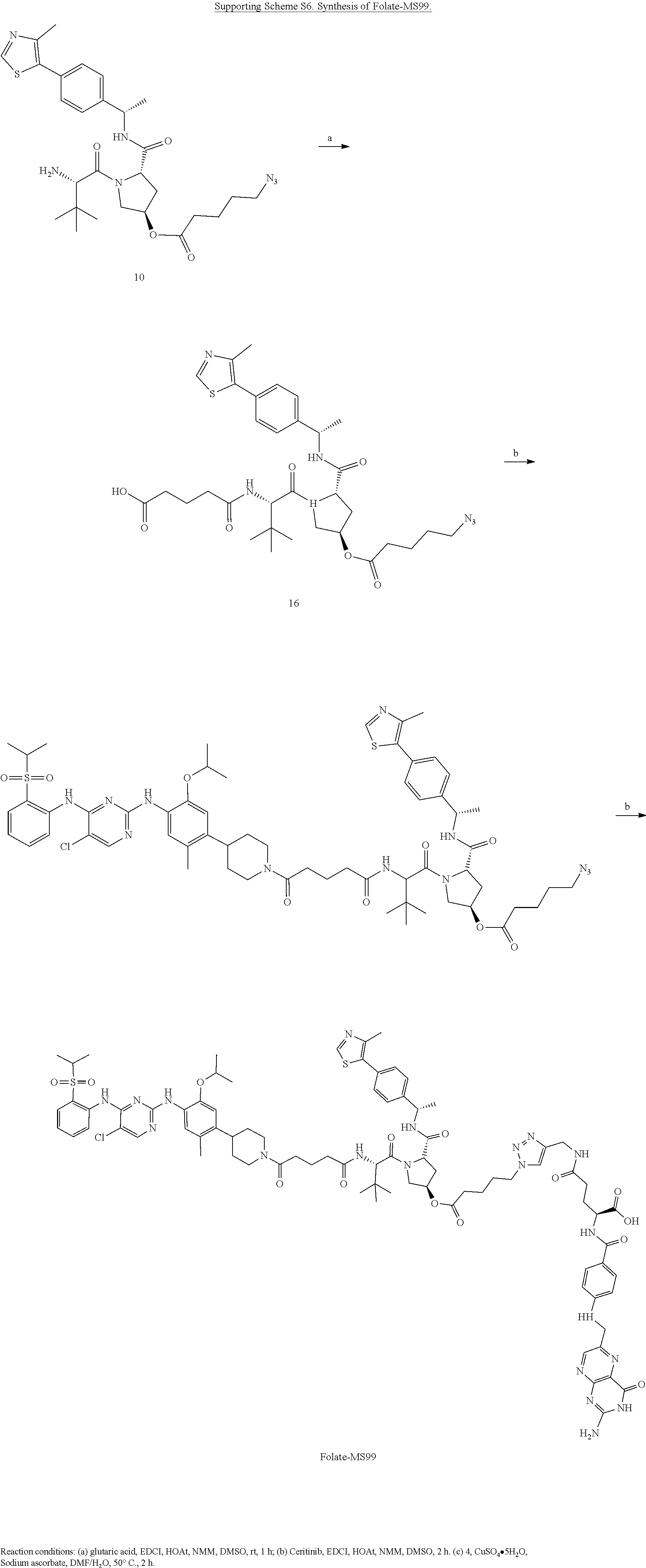

Synthesis of 5-(((S)-1-42S,4R)-4-((5-azidopentanoyl)oxy)-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic acid (16)

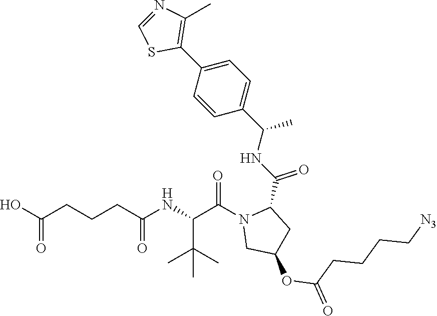 [SMILES: Cc1ncsc1-c1ccc([C@H](C)NC(=O)[C@@H]2C[C@@H](OC(=O)CCCCN=[N+]=[N-])CN2C(=O)[C@@H](NC(=O)CCCC(=O)O)C(C)(C)C)cc1]

To a solution of compound 10 (50.0 mg, 0.073 mmol, 1.0 equiv) in DMSO (2.5 mL) were added glutaric acid (58.1 mg, 0.44 mmol, 6.0 equiv), EDCI (21.1 mg, 0.11 mmol, 1.5 equiv), HOAt (15.0 mg, 0.11 mmol, 1.5 equiv) and NMM (73.7 mg, 0.73 mmol, 10 equiv). After being stirred at room temperature for 1 h, the resulting mixture was purified by preparative HPLC (10%-100% acetonitrile/0.1% TFA in H₂O) to afford compound 16 as white solid (19.0 mg, 38%). ¹H NMR (600 MHz, Methanol-d₄) δ 9.19 (s, 1H), 7.48 (d, J=8.4 Hz, 2H), 7.46 (d, J=8.4 Hz, 2H), 5.38-5.30 (m, 1H), 5.03 (q, J=7.1 Hz, 1H), 4.66-4.57 (m, 1H), 4.52 (s, 1H), 4.20 (d, J=11.7 Hz, 1H), 3.87 (dd, J=11.8, 4.0 Hz, 1H), 3.33 (t, J=6.6 Hz, 2H), 2.53 (s, 3H), 2.46-2.29 (m, 7H), 2.14 (ddd, J=13.9, 9.3, 4.8 Hz, 1H), 1.90 (p, J=7.4 Hz, 2H), 1.75-1.66 (m, 2H), 1.66-1.59 (m, 2H), 1.52 (d, J=7.0 Hz, 3H), 1.07 (s, 9H). ¹³C NMR (151 MHz, Methanol-d₄) δ 175.35, 173.90, 172.97, 171.28, 171.11, 152.50, 145.92, 144.79, 133.10, 129.20, 129.16, 126.41, 73.17, 59.02, 58.04, 53.99, 50.74, 48.81, 34.64, 34.53, 34.11, 33.01, 32.74, 27.89, 25.66, 22.85, 21.62, 20.97, 13.71. ESI m/z=684.5 [M+H]⁺.

Synthesis of (3R,5S)-1-((S)-2-(5-(4-(4-((5-chloro-4-((2-(isopropylsulfonyl)phenyl)amino)pyrimidin-2-yl)amino)-5-isopropoxy-2-methylphenyl)piperidin-1-yl)-5-oxopentanamido)-3,3-dimethylbutanoyl)-5-(((S)-1-(4-(4-methylthiazol-5-yl)phenyl)ethyl)carbamoyl)pyrrolidin-3-yl 5-azidopentanoate (17)

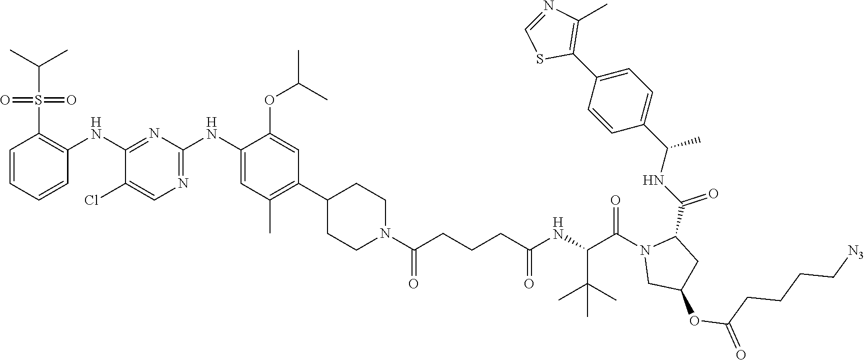 [SMILES: Cc1cc(Nc2ncc(Cl)c(Nc3ccccc3S(=O)(=O)C(C)C)n2)c(OC(C)C)cc1C1CCN(C(=O)CCCC(=O)N[C@H](C(=O)N2C[C@H](OC(=O)CCCCN=[N+]=[N-])C[C@H]2C(=O)N[C@@H](C)c2ccc(-c3scnc3C)cc2)C(C)(C)C)CC1]

To a solution of compound 16 (19.0 mg, 0.028 mmol, 1.0 equiv) in DMSO (1 mL) were added Ceritinib (18.6 mg, 0.033 mmol, 1.2 equiv), EDCI (10.7 mg, 0.056 mmol, 2.0 equiv), HOAt (7.6 mg, 0.056 mmol, 2.0 equiv) and NMM (11.3 mg, 0.112 mmol, 4 equiv). After being stirred at room temperature for 2 h, the resulting mixture was purified by preparative HPLC (10%-100% acetonitrile/0.1% TFA in H₂O) to afford compound 17 as white solid (27.0 mg, 78%). ¹H NMR (600 MHz, Methanol-d₄) δ 8.85 (s, 1H), 8.24 (d, J=8.2 Hz, 1H), 8.11 (s, 1H), 7.88 (dd, J=8.0, 1.6 Hz, 1H), 7.60 (t, J=7.9 Hz, 1H), 7.40 (t, J=7.7 Hz, 1H), 7.37-7.26 (m, 5H), 6.79 (d, J=6.4 Hz, 1H), 5.27-5.18 (m, 1H), 4.96-4.86 (m, 1H), 4.66-4.60 (m, 1H), 4.56-4.50 (m, 1H), 4.50-4.46 (m, 1H), 4.40 (s, 1H), 4.08 (d, J=11.8 Hz, 1H), 4.02 (d, J=13.9 Hz, 1H), 3.83-3.73 (m, 1H), 3.32-3.25 (m, 1H), 3.20 (t, J=6.5 Hz, 2H), 3.15 (tdd, J=13.2, 5.3, 2.6 Hz, 1H), 2.95 (tt, J=12.1, 3.5 Hz, 1H), 2.65 (tt, J=12.9, 2.7 Hz, 1H), 2.39 (s, 3H), 2.33-2.22 (m, 6H), 2.11 (s, 3H), 2.08-2.00 (m, 2H), 1.88-1.79 (m, J=7.2 Hz, 2H), 1.78-1.68 (m, 2H), 1.63-1.54 (m, 3H), 1.53-1.44 (m, 3H), 1.44-1.37 (m, 3H), 1.23-1.14 (m, 12H), 0.98 (s, 9H). ¹³C NMR (151 MHz, Methanol-d₄) δ 174.00, 172.89, 171.73, 171.22, 171.08, 160.30, 157.49, 153.11, 152.01, 148.44, 146.87, 144.96, 144.41, 142.02, 136.30, 134.84, 132.67, 131.35, 129.69, 129.13, 129.07, 127.44, 126.35, 125.86, 125.41, 123.81, 111.74, 105.59, 73.19, 71.26, 59.05, 58.12, 55.62, 53.98, 50.76, 48.78, 46.25, 42.30, 38.25, 34.59, 34.56, 34.41, 33.02, 32.89, 32.82, 32.12, 32.06, 27.91, 25.74, 21.63, 21.06, 20.98, 17.61, 14.17, 14.13. ESI m/z=1223.6 [M+H]⁺.

Synthesis of N²-(4-(((2-amino-4-oxo-3,4-dihydropteridin-6-yl)methyl)amino)benzoyl)-N⁵-((1-(5-(((3R,5S)-1-((S)-2-(5-(4-(4-((5-chloro-4-((2-(isopropylsulfonyl)phenyl)amino)pyrimidin-2-yl)amino)-5-isopropoxy-2-methylphenyl)piperidin-1-yl)-5-oxopentanamido)-3,3-dimethylbutanoyl)-5-(((S)-1-(4-(4-methylthiazol-5-yl)phenyl)ethyl)carbamoyl)pyrrolidin-3-yl)oxy)-5-oxopentyl)-1H-1,2,3-triazol-4-yl)methyl)-L-glutamine (Folate-MS99)

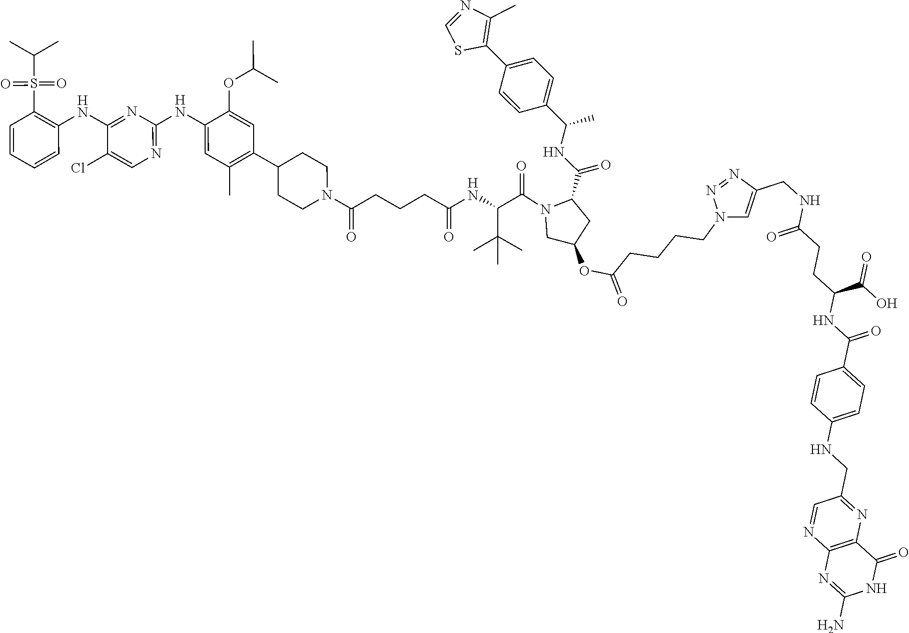 [SMILES: Cc1cc(Nc2ncc(Cl)c(Nc3ccccc3S(=O)(=O)C(C)C)n2)c(OC(C)C)cc1C1CCN(C(=O)CCCC(=O)N[C@H](C(=O)N2C[C@H](OC(=O)CCCCn3cc(CNC(=O)CC[C@H](NC(=O)c4ccc(NCc5cnc6nc(N)[nH]c(=O)c6n5)cc4)C(=O)O)nn3)C[C@H]2C(=O)N[C@@H](C)c2ccc(-c3scnc3C)cc2)C(C)(C)C)CC1]

To a solution of compound 17 (31.0 mg, 0.025 mmol, 1.0 equiv) in DMF (2.0 mL)/water (1.0 mL) were added compound 4 (14.4 mg, 0.030 mmol, 1.2 equiv), sodium ascorbate (2.0 mg, 0.010 mmol, 0.4 equiv) and CuSO₄·5H₂O (1.2 mg, 0.005 mmol, 0.2 equiv) at room temperature. The reaction mixture was heated to 50° C. After being stirred at 50° C. for 2 h, the resulting mixture was purified by preparative HPLC (10%-100% acetonitrile/0.1% TFA in H₂O) to afford Folate-MS99 as light yellow solid (19.2 mg, 45%). ¹H NMR (600 MHz, DMSO-d₆) δ 9.54 (s, 1H), 8.92 (s, 1H), 8.64 (s, 1H), 8.38-8.21 (m, 5H), 8.14 (d, J=7.6 Hz, 1H), 7.85-7.76 (m, 3H), 7.68-7.56 (m, 3H), 7.40 (s, 1H), 7.39-7.26 (m, 5H), 6.75 (d, J=8.7 Hz, 1H), 6.58 (d, J=8.4 Hz, 2H), 5.13 (s, 1H), 4.86-4.81 (m, 1H), 4.53-4.44 (m, 5H), 4.39 (t, J=8.4 Hz, 1H), 4.32-4.14 (m, 5H), 3.92 (d, J=11.7 Hz, 1H), 3.88 (d, J=12.9 Hz, 1H), 3.71-3.63 (m, 1H), 3.37 (p, J=6.8 Hz, 1H), 3.03 (t, J=12.7 Hz, 1H), 2.87-2.76 (m, 1H), 2.52 (t, J=12.8 Hz, 1H), 2.38 (s, 3H), 2.33-2.10 (m, 8H), 2.10-1.98 (m, 5H), 1.96-1.81 (m, 2H), 1.78-1.56 (m, 6H), 1.55-1.34 (m, 4H), 1.34-1.26 (m, 3H), 1.17-1.05 (m, 12H), 0.89 (s, 9H). ¹³C NMR (151 MHz, DMSO-d₆) δ 174.29, 172.86, 172.75, 172.00, 170.46, 170.40, 170.35, 166.79, 160.37, 157.31, 155.90, 153.75, 153.49, 152.00, 151.11, 151.01, 148.56, 148.19, 147.21, 145.43, 145.02, 139.85, 138.11, 135.35, 131.65, 131.48, 130.18, 129.45, 129.32, 128.45, 127.04, 126.97, 126.82, 126.73, 125.82, 124.85, 124.71, 124.42, 123.07, 121.91, 112.30, 111.70, 104.82, 73.56, 71.14, 58.70, 57.71, 55.24, 54.02, 52.57, 49.35, 48.29, 46.27, 46.11, 42.19, 40.49, 38.21, 34.88, 34.85, 34.72, 33.20, 33.10, 32.47, 32.27, 29.47, 26.94, 26.86, 26.78, 22.88, 22.31, 21.88, 21.62, 18.88, 16.40, 15.28. HRMS (ESI-TOF) calcd for C₈₃H₁₀₂ClN₂₀O₁₄S₂⁺[M+H]⁺1701.7009, found 1701.7003. HPLC>98%, t_{R}=4.90 min.

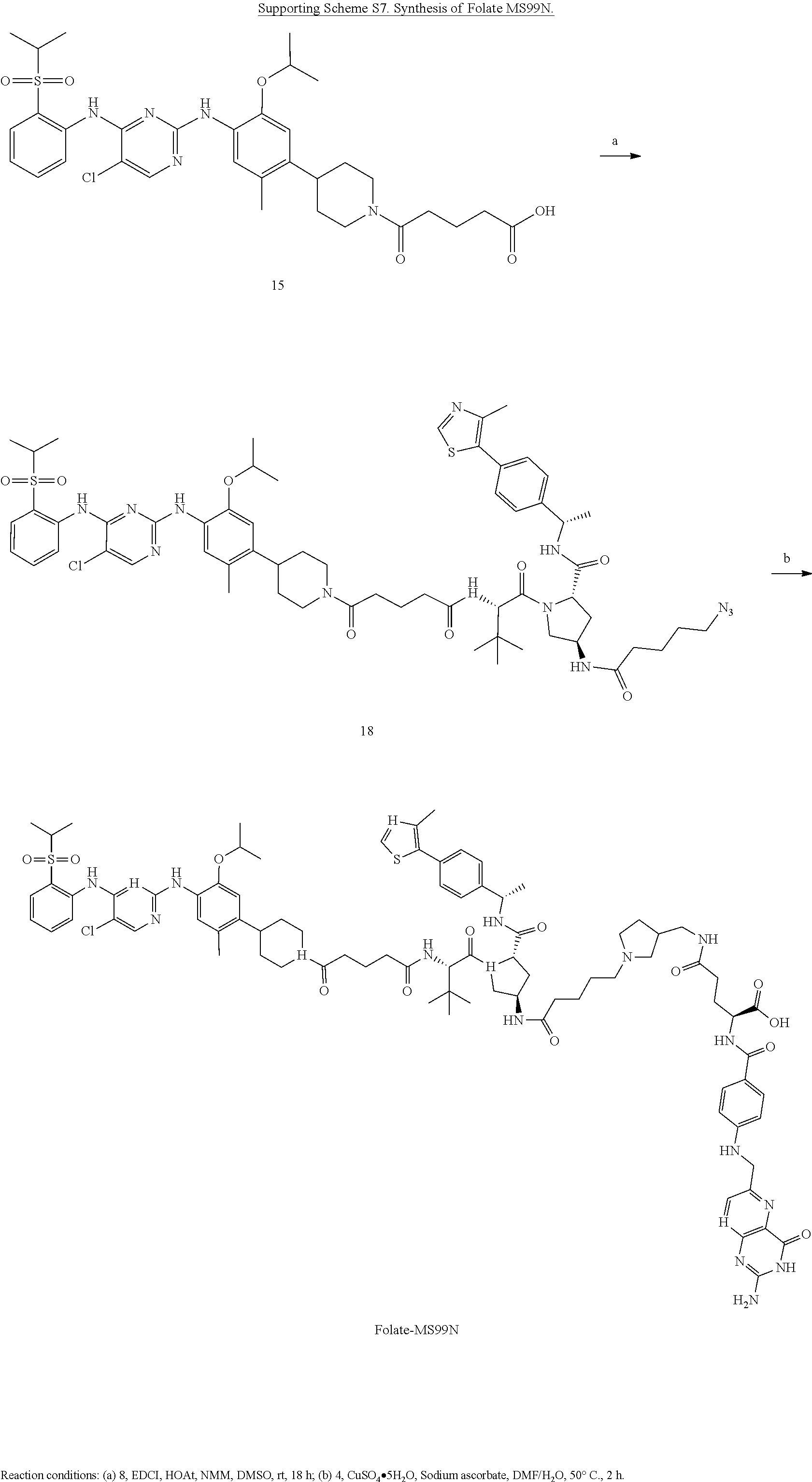

Synthesis of (2S,4R)-4-(5-azidopentanamido)-1-((S)-2-(5-(4-(4-((5-chloro-4-((2-(isopropylsulfonyl)phenyl)amino)pyrimidin-2-yl)amino)-5-isopropoxy-2-methylphenyl)piperidin-1-yl)-5-oxopentanamido)-3,3-dimethylbutanoyl)-N-((S)-1-(4-(4-methylthiazol-5-yl)phenyl)ethyl)pyrrolidine-2-carboxamide (18)

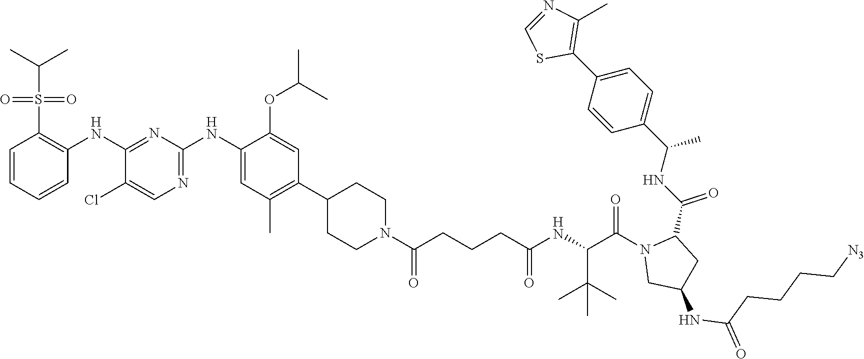

To a solution of compound 15 (33.6 mg, 0.05 mmol, 1.0 equiv) in DMSO (2.5 mL) were added compound 8 (35.8 mg, 0.053 mmol, 1.05 equiv), EDCI (19.2 mg, 0.10 mmol, 2.0 equiv), HOAt (13.6 mg, 0.10 mmol, 2.0 equiv) and NMM (20.2 mg, 0.20 mmol, 4 equiv). After being stirred at room temperature for 18 h, the resulting mixture was purified by preparative HPLC (10%-100% acetonitrile/0.1% TFA in H₂O) to afford compound 18 as white solid (30.1 mg, 49%). ¹H NMR (600 MHz, Methanol-d₄) δ 8.96 (s, 1H), 8.34 (s, 1H), 8.21 (s, 1H), 8.03-7.92 (m, 1H), 7.74-7.64 (m, 1H), 7.55-7.33 (m, 6H), 6.88 (d, J=5.6 Hz, 1H), 5.04-4.95 (m, 1H), 4.74 (d, J=12.7 Hz, 1H), 4.67-4.56 (m, 2H), 4.45 (s, 1H), 4.14 (d, J=13.4 Hz, 1H), 4.01-3.92 (m, 1H), 3.92-3.82 (m, 1H), 3.44-3.20 (m, 4H), 3.10-2.99 (m, 1H), 2.75 (t, J=12.5 Hz, 1H), 2.60-2.33 (m, 8H), 2.32-2.16 (m, 6H), 2.12 (dt, J=13.4, 7.2 Hz, 1H), 2.02-1.90 (m, 2H), 1.89-1.77 (m, 2H), 1.73-1.54 (m, 6H), 1.52-1.44 (m, 3H), 1.35-1.18 (m, 12H), 1.10 (s, 9H). ¹³C NMR (151 MHz, Methanol-d₄) δ 174.45, 174.25, 171.78, 171.26, 171.21, 160.36, 157.25, 153.83, 151.76, 147.25, 145.82, 144.27, 144.22, 141.22, 136.57, 134.80, 131.30, 129.91, 129.58, 129.12, 129.07, 127.31, 126.26, 125.55, 125.35, 111.70, 105.52, 71.27, 58.69, 58.47, 55.56, 53.15, 50.72, 49.33, 48.72, 48.23, 46.24, 42.29, 38.21, 34.89, 34.51, 34.06, 32.84, 32.02, 28.00, 25.68, 22.64, 21.43, 21.05, 21.00, 20.95, 17.58, 14.27, 14.09. ESI m/z=1222.5 [M+H]⁺.

Synthesis of N²-(4-(((2-amino-4-oxo-3,4-dihydropteridin-6-yl)methyl)amino)benzoyl)-N⁵-((1-(5-(((3R,5S)-1-((S)-2-(5-(4-(4-((5-chloro-4-((2-(isopropylsulfonyl)phenyl)amino)pyrimidin-2-yl)amino)-5-isopropoxy-2-methylphenyl)piperidin-1-yl)-5-oxopentanamido)-3,3-dimethylbutanoyl)-5-(((S)-1-(4-(4-methylthiazol-5-yl)phenyl)ethyl)carbamoyl)pyrrolidin-3-yl)amino)-5-oxopentyl)-1H-1,2,3-triazol-4-yl)methyl)-L-glutamine (Folate-MS99N)

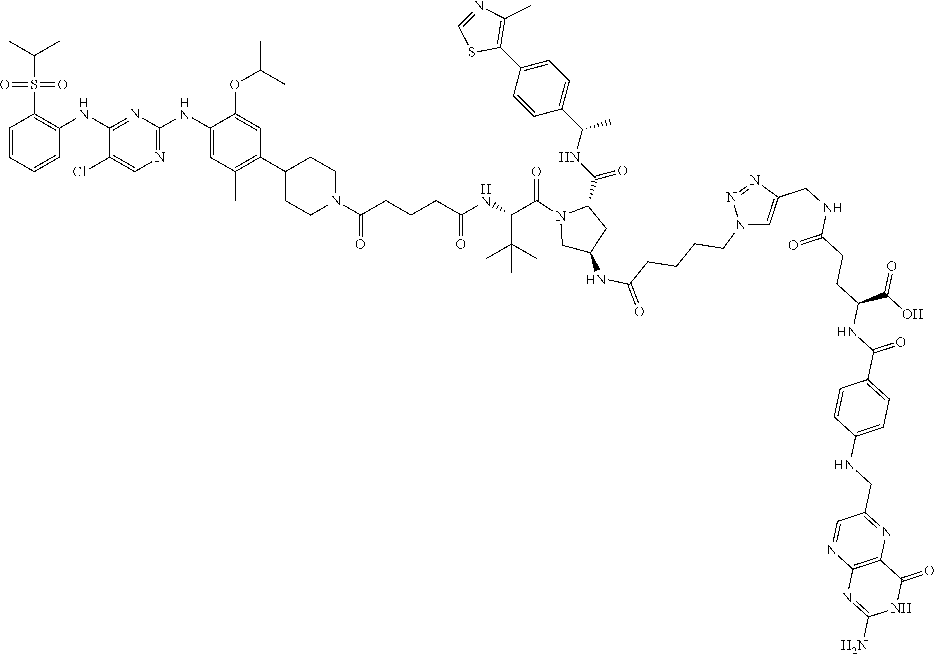 [SMILES: Cc1cc(Nc2ncc(Cl)c(Nc3ccccc3S(=O)(=O)C(C)C)n2)c(OC(C)C)cc1C1CCN(C(=O)CCCC(=O)N[C@H](C(=O)N2C[C@H](NC(=O)CCCCn3cc(CNC(=O)CC[C@H](NC(=O)c4ccc(NCc5cnc6nc(N)[nH]c(=O)c6n5)cc4)C(=O)O)nn3)C[C@H]2C(=O)N[C@@H](C)c2ccc(-c3scnc3C)cc2)C(C)(C)C)CC1]

To a solution of compound 18 (24.4 mg, 0.02 mmol, 1.0 equiv) in DMF (2.0 mL)/water (1.0 mL) were added compound 4 (14.4 mg, 0.03 mmol, 1.5 equiv), sodium ascorbate (5.9 mg, 0.03 mmol, 1.5 equiv) and CuSO₄·5H₂O (5.0 mg, 0.02 mmol, 1.0 equiv) at room temperature. The reaction mixture was heated to 50° C. After being stirred at 50° C. for 2 h, the resulting mixture was purified by preparative HPLC (10%-100% acetonitrile/0.1% TFA in H₂O) to afford Folate-MS99N as light yellow solid (17.5 mg, 51%). ¹H NMR (600 MHz, DMSO-d₆) δ 9.53 (s, 1H), 8.92 (s, 1H), 8.63 (s, 1H), 8.32 (d, J=8.4 Hz, 1H), 8.29-8.17 (m, 4H), 8.13 (d, J=7.6 Hz, 1H), 7.96 (dd, J=6.7, 2.5 Hz, 1H), 7.84 (d, J=8.1 Hz, 1H), 7.81-7.74 (m, 2H), 7.66-7.53 (m, 3H), 7.41 (s, 1H), 7.38-7.27 (m, 5H), 6.76 (d, J=6.5 Hz, 1H), 6.58 (d, J=8.7 Hz, 2H), 4.88-4.79 (m, 1H), 4.54-4.47 (m, 2H), 4.46 (s, 2H), 4.44-4.41 (m, 1H), 4.34 (dd, J=8.3, 4.5 Hz, 1H), 4.26-4.12 (m, 6H), 3.92-3.84 (m, 1H), 3.72-3.64 (m, 1H), 3.55-3.49 (m, 1H), 3.42-3.31 (m, 1H), 3.04 (t, J=12.7 Hz, 1H), 2.89-2.76 (m, 1H), 2.58-2.51 (m, 1H), 2.38 (s, 3H), 2.31-2.12 (m, 6H), 2.09-1.94 (m, 7H), 1.90-1.79 (m, 2H), 1.74-1.57 (m, 6H), 1.55-1.45 (m, 1H), 1.43-1.33 (m, 3H), 1.32-1.25 (m, 3H), 1.18-1.05 (m, 12H), 0.90 (s, 9H). ¹³C NMR (151 MHz, DMSO-d₆) δ 174.29, 173.02, 172.26, 172.02, 170.69, 170.48, 170.26, 166.80, 159.91, 156.38, 153.17, 152.03, 151.84, 151.06, 150.28, 148.36, 148.13, 147.33, 145.39, 145.05, 140.24, 137.82, 135.37, 131.69, 131.54, 130.16, 129.58, 129.46, 129.32, 128.42, 127.06, 127.02, 126.81, 126.52, 126.26, 125.38, 125.20, 124.57, 123.08, 121.96, 112.31, 111.74, 104.88, 71.19, 71.17, 58.38, 57.55, 57.49, 55.21, 53.17, 52.58, 49.46, 48.63, 48.25, 46.23, 46.11, 42.20, 40.45, 38.23, 34.91, 34.87, 34.73, 33.08, 32.45, 32.28, 29.78, 26.93, 26.85, 22.83, 22.54, 22.27, 21.82, 18.85, 16.36, 15.26. HRMS calcd for C₈₃H₁₀₃ClN₂₁O₁₃S₂⁺[M+H]⁺ 1700.7169, found 1700.7161. HPLC>97%, t_{R}=4.87 min.

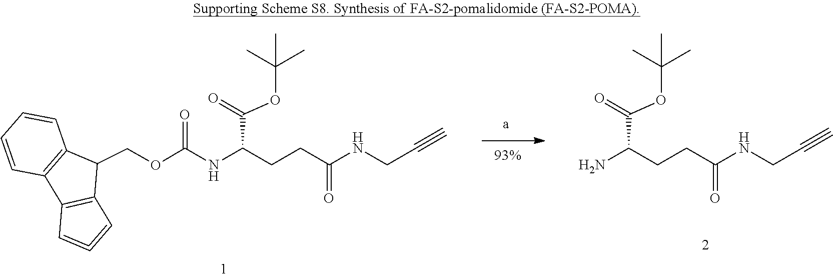

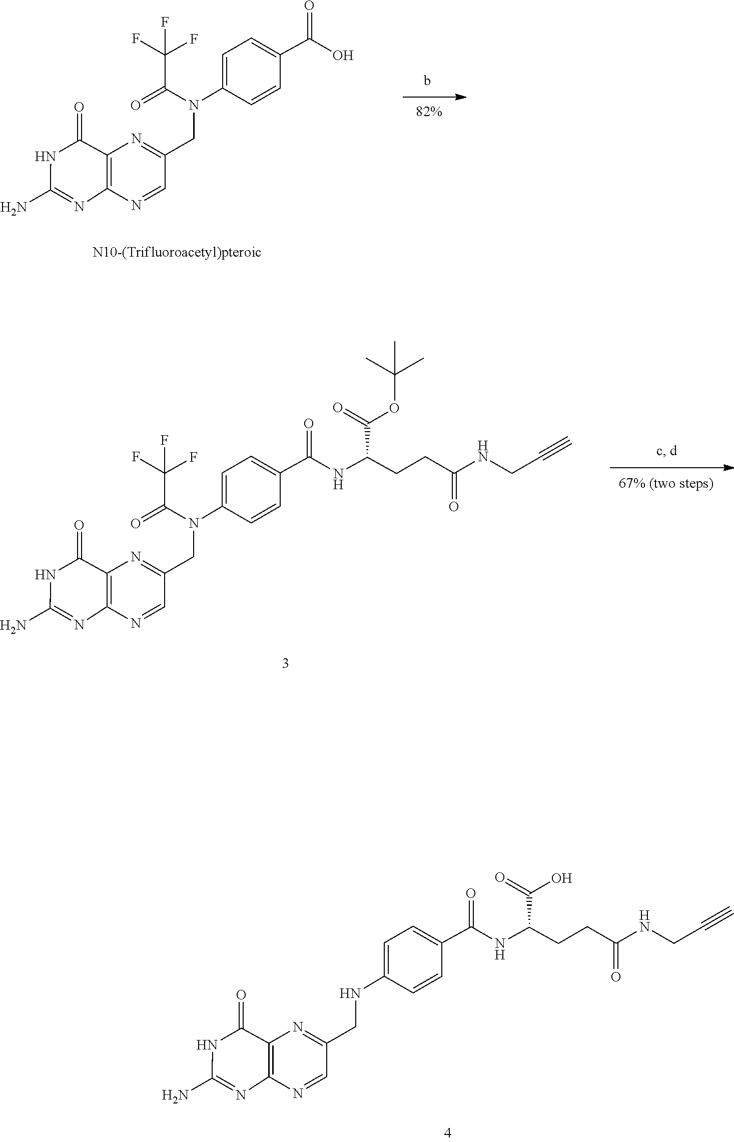

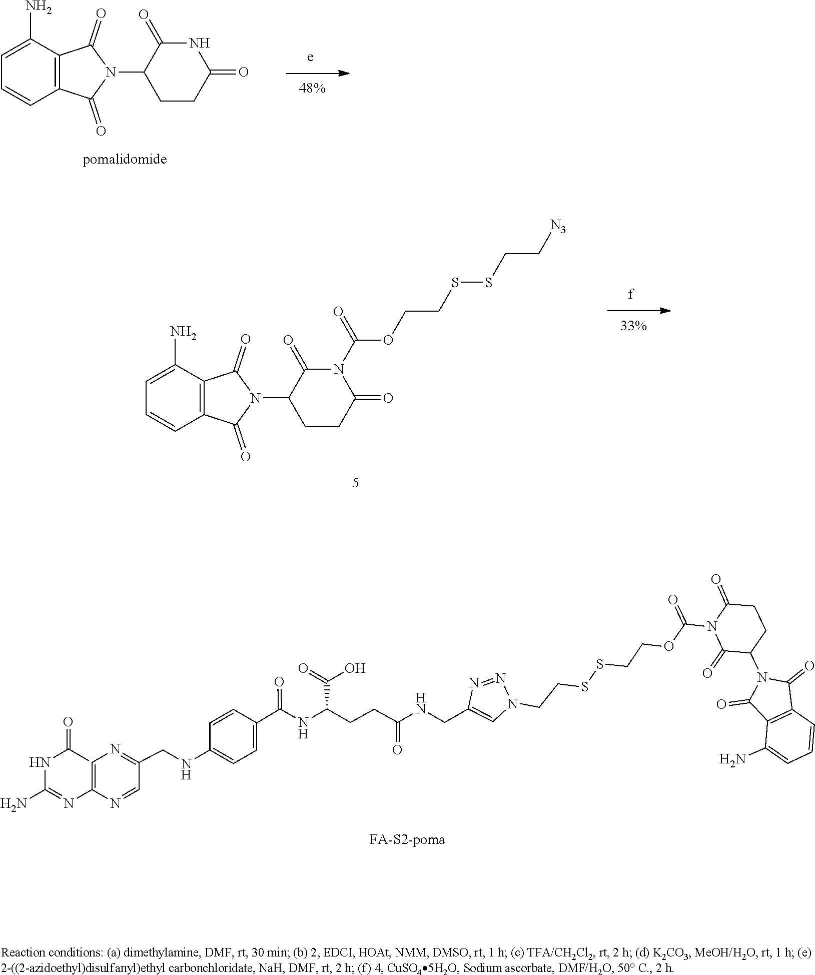 [SMILES: Nc1cccc2c1C(=O)N(C1CCC(=O)NC1=O)C2=O ; [N-]=[N+]=NCCSSCCOC(=O)N1C(=O)CCC(N2C(=O)c3cccc(N)c3C2=O)C1=O ; CC(=O)N/C(N)=N\c1cnc(CNc2ccc(C(=O)N[C@@H](CCC(=O)NCc3cn(CCSSCCOC(=O)N4C(=O)CCC(N5C(=O)c6cccc(N)c6C5=O)C4=O)nn3)C(=O)O)cc2)cn1]

Synthesis of tert-butyl N⁵-(prop-2-yn-1-yl)-L-glutaminate (2)

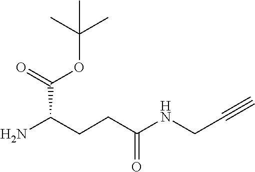 [SMILES: C#CCNC(=O)CC[C@H](N)C(=O)OC(C)(C)C]

To a solution of compound 1 (see reference⁴ for the details of synthesis) (300 mg, 0.65 mmol, 1.0 equiv) in DMF (2 mL) was added dimethylamine (1.62 mL, 2M THF solution, 3.25 mmol, 5.0 equiv) at room temperature. The reaction mixture was stirred at room temperature for 30 min. Then the reaction solution was diluted with ethyl acetate (30 mL) and washed with water (2×30 mL) and brine (30 mL). The organic layer was concentrated under reduced pressure. The crude product was purified by preparative HPLC (10%-100% acetonitrile/0.1% TFA in H₂O). The product containing fractions were concentrated to remove the organic solvent under reduced pressure and then dried by lyophilization to afford compound 2 as colorless solid in TFA salt form (213 mg, 93%). ¹H NMR (600 MHz, Methanol-d₄) δ 4.05-3.93 (m, 3H), 2.59 (t, J=2.5 Hz, 1H), 2.54-2.42 (m, 2H), 2.24-2.09 (m, 2H), 1.54 (s, 9H). ¹³C NMR (151 MHz, Methanol-d₄) δ 172.18, 167.92, 84.03, 79.47, 70.88, 52.61, 30.59, 28.14, 26.73, 25.68. ESI m/z=241.3 [M+H]⁺.

Synthesis of tert-butyl N²-(4-(N-((2-amino-4-oxo-3,4-dihydropteridin-6-yl)methyl)-2,2,2-trifluoroacetamido)benzoyl)-N⁵-(prop-2-yn-1-yl)-L-glutaminate (3)

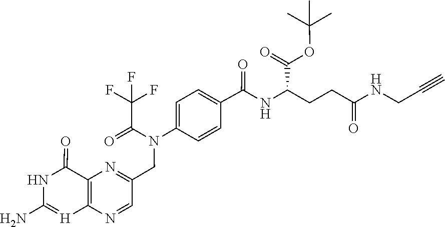

To a solution of N¹⁰-(Trifluoroacetyl)pteroic acid (204.2 mg, 0.5 mmol, 1.0 equiv) in DMSO (4 mL) were added compound 2 (212.6 mg, 0.6 mmol, 1.2 equiv), EDCI (1-ethyl-3-(3-dimethylaminopropyl)carbodiimide) (191.7 mg, 1.0 mmol, 2.0 equiv), HOAt (1-hydroxy-7-azabenzo-triazole) (136.1 mg, 1.0 mmol, 2.0 equiv) and NMM (N-Methylmorpholine) (202.2 mg, 2.0 mmol, 4.0 equiv). After being stirred at room temperature for 1 h, the resulting mixture was purified by preparative HPLC (10%-100% acetonitrile/0.1% TFA in H₂O). The product containing fractions were concentrated to remove the organic solvent under reduced pressure and then dried by lyophilization to afford compound 3 as brown solid (258.5 mg, 82%). ¹H NMR (600 MHz, DMSO-d₆) δ 8.77 (d, J=7.4 Hz, 1H), 8.73 (s, 1H), 8.31 (t, J=5.5 Hz, 1H), 7.92 (d, J=8.2 Hz, 2H), 7.66 (d, J=8.1 Hz, 2H), 5.18 (s, 2H), 4.31-4.22 (m, 1H), 3.92-3.78 (m, 2H), 3.07 (t, J=2.5 Hz, 1H), 2.34-2.19 (m, 2H), 2.08-2.02 (m, 1H), 1.98-1.88 (m, 1H), 1.41 (s, 9H). ¹³C NMR (151 MHz, DMSO-d₆) δ 171.13, 171.02, 165.71, 159.88, 155.72 (q, J=34.7 Hz), 153.27, 152.31, 148.95, 145.91, 141.62, 134.54, 128.70, 128.49, 128.13, 116.12 (q, J=288.4 Hz), 81.19, 80.68, 72.91, 53.82, 53.16, 31.49, 27.84, 27.68, 26.21. ESI m/z=631.3 [M+H]⁺.

Synthesis of N²-(4-(((2-amino-4-oxo-3,4-dihydropteridin-6-yl)methyl)amino)benzoyl)-N⁵-(prop-2-yn-1-yl)-L-glutamine (4)

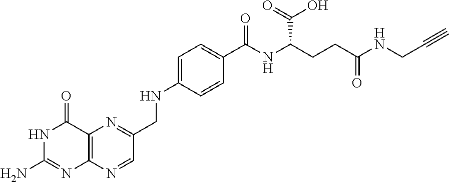 [SMILES: C#CCNC(=O)CC[C@H](NC(=O)c1ccc(NCc2cnc3nc(N)[nH]c(=O)c3n2)cc1)C(=O)O]

To a suspension of compound 3 (258.5 mg, 0.41 mmol) in dichloromethane (2 mL) was added TFA (trifluoroacetic acid) (2 mL) at room temperature. After being stirred at room temperature for 2 h, the resulting mixture was concentrated under reduced pressure and purified by preparative HPLC (10%-100% acetonitrile/0.1% TFA in H₂O) to afford the intermediate as brown solid. ESI m/z=575.3 [M+H⁺]. To a suspension of the obtained brown solid in methanol (3 mL) was dropwise added a solution of K₂CO₃ (169.7 mg) in water (1 mL) at room temperature. The reaction mixture was stirred at room temperature for 1 h and then concentrated under reduced pressure to remove methanol. The resulting solution was diluted with water (3 mL) and then pH was adjusted with hydrochloric acid (HCl, 3N) to 2-3. The suspension was filtered and the solid cake was washed with water. After being dried under reduced pressure, afforded compound 4 as brown solid (132.0 mg, 67% yield for two step). ¹H NMR (600 MHz, DMSO-d₆) δ 12.46 (s, 1H), 11.41 (s, 1H), 8.65 (s, 1H), 8.27 (t, J=5.5 Hz, 1H), 8.16 (d, J=7.6 Hz, 1H), 7.65 (d, J=8.8 Hz, 2H), 6.93 (s, 1H), 6.64 (d, J=8.8 Hz, 2H), 4.52-4.45 (m, 2H), 4.27 (ddd, J=9.8, 7.5, 4.7 Hz, 1H), 3.88-3.77 (m, 2H), 3.06 (t, J=2.5 Hz, 1H), 2.26-2.15 (m, 2H), 2.10-1.99 (m, 1H), 1.95-1.84 (m, 1H). ¹³C NMR (151 MHz, DMSO-d₆) δ 174.27, 171.82, 166.83, 161.53, 156.55, 154.25, 151.23, 149.14, 149.05, 129.46, 128.41, 121.80, 111.66, 81.68, 73.34, 52.63, 46.37, 32.18, 28.29, 26.87. ESI m/z=479.4 [M+H]⁺.

Synthesis of 2-((2-azidoethyl)disulfanyl)ethyl 3-(4-amino-1,3-dioxoisoindolin-2-yl)-2,6-dioxopiperidine-1-carboxylate (5).

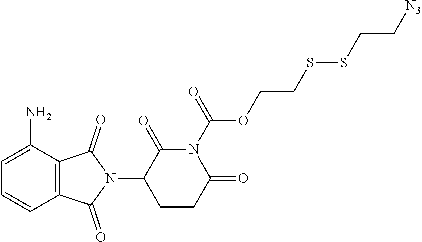 [SMILES: [N-]=[N+]=NCCSSCCOC(=O)N1C(=O)CCC(N2C(=O)c3cccc(N)c3C2=O)C1=O]

To a solution of pomalidomide (55.0 mg, 0.2 mmol, 1.0 equiv) in DMF (2 mL) was added NaH (9.6 mg, 60% in mineral oil, 0.24 mmol, 1.2 equiv) at 0° C. After stirring for 10 min, 2-((2-azidoethyl)disulfanyl)ethyl carbonochloridate (see reference⁵ for the details of synthesis) (58.0 mg, 0.24 mmol, 1.2 equiv) was added to the mixture at 0° C. The reaction mixture was then warmed up to room temperature and stirred for additional 2 h. The resulting mixture was purified by preparative HPLC (10%-100% acetonitrile/0.1% TFA in H₂O). The product containing fractions were concentrated to remove the organic solvent under reduced pressure and then dried by lyophilization to afford compound 5 as yellow solid (45.9 mg, 48%). ¹H NMR (600 MHz, DMSO-d₆) δ 7.49 (t, J=7.7 Hz, 1H), 7.06-7.00 (m, 2H), 6.56 (s, 2H), 5.37 (dd, J=12.9, 5.4 Hz, 1H), 4.65-4.48 (m, 2H), 3.60 (t, J=6.4 Hz, 2H), 3.18-3.02 (m, 3H), 2.95 (t, J=6.5 Hz, 2H), 2.83 (d, J=18.1 Hz, 1H), 2.68-2.55 (m, 1H), 2.19-2.04 (m, 1H). ESI m/z=501.1 [M+Na]⁺.

Synthesis of N⁵-((1-(2-((2-((3-(4-amino-1,3-dioxoisoindolin-2-yl)-2,6-dioxopiperidine-1-carbonyl)oxy)ethyl)disulfanyl)ethyl)-1H-1,2,3-triazol-4-yl)methyl)-N²-(4-(((2-amino-4-oxo-3,4-dihydropteridin-6-yl)methyl)amino)benzoyl)-L-glutamine (FA-S2-poma).

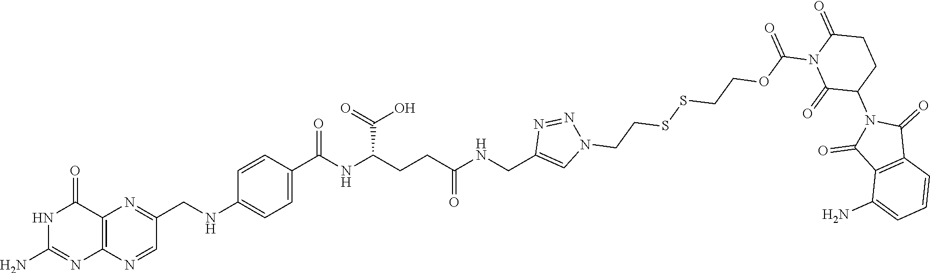 [SMILES: Nc1nc2ncc(CNc3ccc(C(=O)N[C@@H](CCC(=O)NCc4cn(CCSSCCOC(=O)N5C(=O)CCC(N6C(=O)c7cccc(N)c7C6=O)C5=O)nn4)C(=O)O)cc3)nc2c(=O)[nH]1]

To a solution of compound 5 (24.0 mg, 0.05 mmol, 1.0 equiv) in DMF (1.6 mL)/water (0.8 mL) were added compound 4 (28.8 mg, 0.06 mmol, 1.2 equiv), sodium ascorbate (2.9 mg, 0.015 mmol, 0.3 equiv) and CuSO₄·5H₂O (1.3 mg, 0.007 mmol, 0.15 equiv) at room temperature. The reaction mixture was heated to 50° C. After being stirred at 50° C. for 2 h, the resulting mixture was purified by preparative HPLC (10%-100% acetonitrile/0.1% TFA in H₂O). The product containing fractions were concentrated to remove the organic solvent under reduced pressure at room temperature and then dried by lyophilization to afford FA-S2-poma as yellow solid (15.9 mg, 33%). ¹H NMR (600 MHz, DMSO-d₆) δ 8.61 (s, 1H), 8.29 (t, J=5.8 Hz, 1H), 8.13 (d, J=7.6 Hz, 1H), 7.84 (s, 1H), 7.59 (d, J=8.4 Hz, 2H), 7.48-7.35 (m, 1H), 7.07-6.89 (m, 2H), 6.58 (d, J=8.4 Hz, 2H), 6.54-6.42 (m, 2H), 5.30 (dd, J=12.9, 5.4 Hz, 1H), 4.58-4.38 (m, 6H), 4.32-4.10 (m, 3H), 3.14 (t, J=6.6 Hz, 2H), 3.05 (ddd, J=18.4, 13.7, 5.5 Hz, 1H), 2.99 (t, J=6.3 Hz, 2H), 2.76 (dt, J=17.7, 3.5 Hz, 1H), 2.54 (qd, J=13.3, 4.5 Hz, 1H), 2.24-2.12 (m, 2H), 2.09-1.98 (m, 2H), 1.89-1.80 (m, 1H). ¹³C NMR (151 MHz, DMSO-d₆) δ 174.29, 172.03, 170.61, 168.73, 168.32, 167.62, 166.87, 161.45, 156.06, 154.25, 151.22, 150.94, 149.35, 149.04, 147.30, 145.63, 136.10, 132.32, 129.46, 123.40, 122.36, 121.77, 111.67, 111.63, 108.77, 67.22, 52.54, 48.75, 48.38, 46.36, 37.43, 35.87, 34.66, 32.25, 31.19, 26.92, 21.73. HRMS (ESI-TOF) calcd for C₄₀H₄₁N₁₄O₁₁S₂⁺[M+H]⁺ 957.2515, found 957.2502. HPLC>98%, t_{R}=3.80 min.

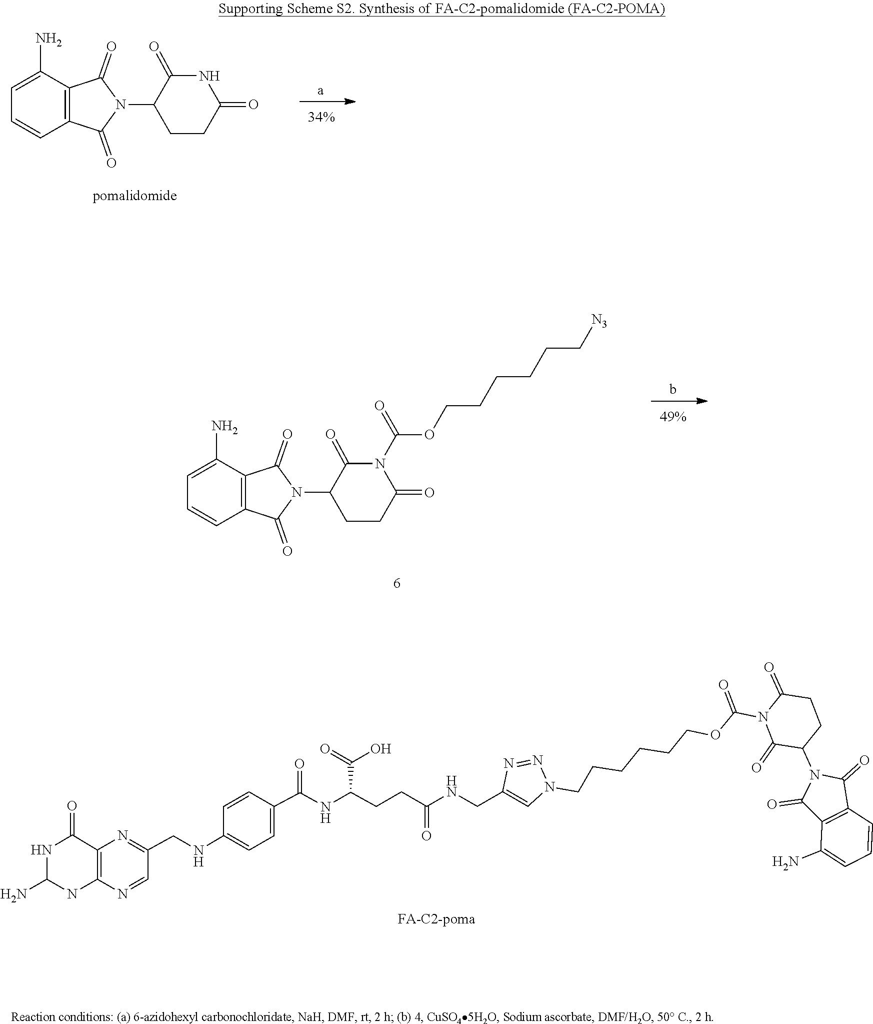

Synthesis of 6-azidohexyl 3-(4-amino-1,3-dioxoisoindolin-2-yl)-2,6-dioxopiperidine-1-carboxylate (6)

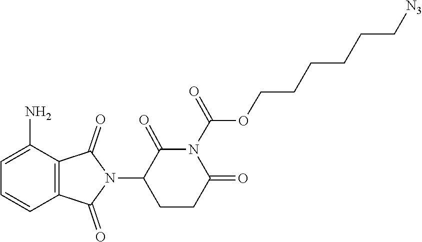 [SMILES: [N-]=[N+]=NCCCCCCOC(=O)N1C(=O)CCC(N2C(=O)c3cccc(N)c3C2=O)C1=O]

To a solution of pomalidomide (109.3 mg, 0.4 mmol, 1.0 equiv) in DMF (2 mL) was added NaH (19.2 mg, 60% in mineral oil, 0.48 mmol, 1.2 equiv) at 0° C. After stirring for 10 min, 6-azidohexyl carbonochloridate (see reference⁵ for the details of synthesis) (99.0 mg, 0.48 mmol, 1.2 equiv) was added to the mixture at 0° C. The reaction mixture was then warmed up to room temperature and stirred for additional 2 h. The resulting mixture was purified by preparative HPLC (10%-100% acetonitrile/0.1% TFA in H₂O). The product containing fractions were concentrated to remove the organic solvent under reduced pressure and then dried by lyophilization to afford compound 6 as yellow solid (60.2 mg, 34%). ¹H NMR (600 MHz, DMSO-d₆) δ 7.53-7.45 (m, 1H), 7.06-6.99 (m, 2H), 6.56 (s, 2H), 5.36 (dd, J=12.9, 5.3 Hz, 1H), 4.47-4.26 (m, 2H), 3.30 (t, J=6.9 Hz, 2H), 3.16-3.06 (m, 1H), 2.86-2.76 (m, 1H), 2.60 (qd, J=13.3, 4.4 Hz, 1H), 2.14-2.07 (m, 1H), 1.72-1.63 (m, 2H), 1.56-1.48 (m, 2H), 1.42-1.29 (m, 4H). ESI m/z=465.2 [M+Na]⁺.

Synthesis of N⁵-((1-(6-((3-(4-amino-1,3-dioxoisoindolin-2-yl)-2,6-dioxopiperidine-1-carbonyl)oxy)hexyl)-1H-1,2,3-triazol-4-yl)methyl)-N²-(4-(((2-amino-4-oxo-3,4-dihydropteridin-6-yl)methyl)amino)benzoyl)-L-glutamine (FA-C2-poma).

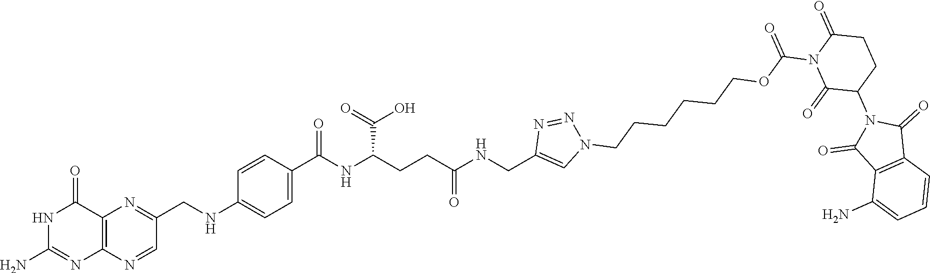 [SMILES: Nc1nc2ncc(CNc3ccc(C(=O)N[C@@H](CCC(=O)NCc4cn(CCCCCCOC(=O)N5C(=O)CCC(N6C(=O)c7cccc(N)c7C6=O)C5=O)nn4)C(=O)O)cc3)nc2c(=O)[nH]1]

To a solution of compound 6 (15.9 mg, 0.036 mmol, 1.2 equiv) in DMF (1.6 mL)/water (0.8 mL) were added compound 4 (14.4 mg, 0.03 mmol, 1.0 equiv), sodium ascorbate (2.4 mg, 0.012 mmol, 0.4 equiv) and CuSO₄·5H₂O (1.5 mg, 0.006 mmol, 0.2 equiv) at room temperature. The reaction mixture was heated to 50° C. After being stirred at 50° C. for 2 h, the resulting mixture was purified by preparative HPLC (10%-100% acetonitrile/0.1% TFA in H₂O). The product containing fractions were concentrated to remove the organic solvent under reduced pressure at room temperature and then dried by lyophilization to afford FA-C2-poma as yellow solid (13.6 mg, 49%). ¹H NMR (600 MHz, DMSO-d₆) δ 8.62 (s, 1H), 8.26 (t, J=5.7 Hz, 1H), 8.13 (d, J=7.7 Hz, 1H), 7.78 (s, 1H), 7.59 (d, J=8.6 Hz, 2H), 7.40 (t, J=7.8 Hz, 1H), 6.99-6.90 (m, 2H), 6.58 (d, J=8.0 Hz, 2H), 5.28 (dd, J=13.1, 5.0 Hz, 1H), 4.45 (s, 2H), 4.35-4.13 (m, 7H), 3.11-2.97 (m, 1H), 2.79-2.71 (m, 1H), 2.59-2.48 (m, 1H), 2.28-2.13 (m, 2H), 2.10-1.97 (m, 2H), 1.94-1.81 (m, 1H), 1.76-1.64 (m, 2H), 1.61-1.49 (m, 2H), 1.37-1.25 (m, 2H), 1.23-1.10 (m, 2H). NMR (151 MHz, DMSO-d₆) δ 174.31, 171.98, 170.64, 168.73, 168.35, 167.61, 166.82, 161.00, 154.47, 153.97, 151.18, 150.94, 150.02, 148.83, 147.31, 145.41, 136.07, 132.34, 129.45, 123.05, 122.34, 121.85, 111.68, 111.59, 108.78, 69.61, 52.55, 49.58, 48.74, 46.33, 34.73, 32.26, 31.19, 30.00, 27.94, 26.92, 25.76, 24.76, 21.79. HRMS (ESI-TOF) calcd for C₄₂H₄₅N₁₄O₁₁⁺ [M+H]⁺ 921.3387, found 921.3382. HPLC>98%, t_{R}=3.80 min.

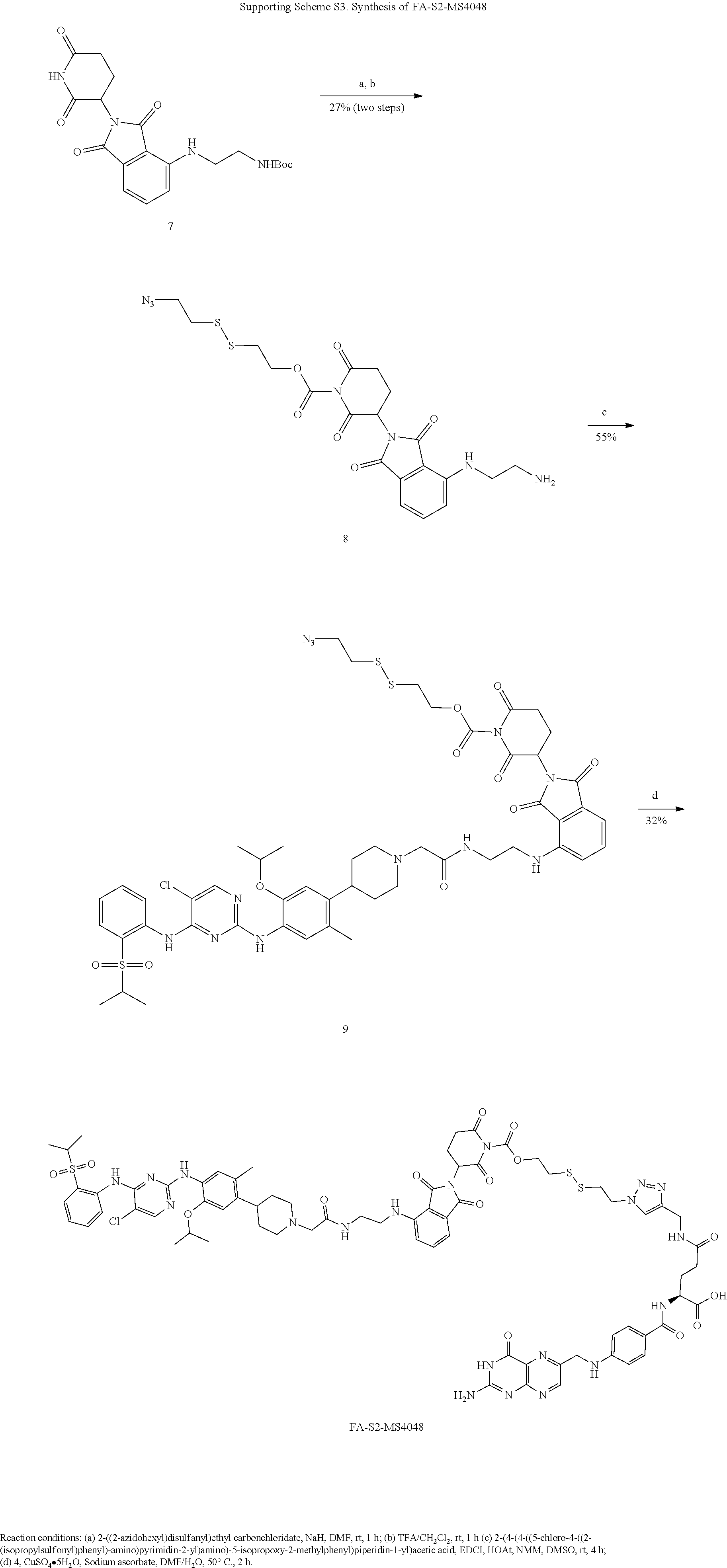 [SMILES: CC(C)(C)OC(=O)NCCNc1cccc2c1C(=O)N(C1CCC(=O)NC1=O)C2=O ; [N-]=[N+]=NCCSSCCOC(=O)N1C(=O)CCC(N2C(=O)c3cccc(NCCN)c3C2=O)C1=O ; Cc1cc(Nc2ncc(Cl)c(Nc3ccccc3S(=O)(=O)C(C)C)n2)c(OC(C)C)cc1C1CCN(CC(=O)NCCNc2cccc3c2C(=O)N(C2CCC(=O)N(C(=O)OCCSSCCN=[N+]=[N-])C2=O)C3=O)CC1 ; Cc1cc(Nc2ncc(Cl)c(Nc3ccccc3S(=O)(=O)C(C)C)n2)c(OC(C)C)cc1C1CCN(CC(=O)NCCNc2cccc3c2C(=O)N(C2CCC(=O)N(C(=O)OCCSSCCn4cc(CNC(=O)CC[C@H](NC(=O)c5ccc(NCc6cnc7nc(N)[nH]c(=O)c7n6)cc5)C(=O)O)nn4)C2=O)C3=O)CC1]

Synthesis of 2-((2-azidoethyl)disulfanyl)ethyl 3-(4-((2-aminoethyl)amino)-1,3-dioxoisoindolin-2-yl)-2,6-dioxopiperidine-1-carboxylate (8)

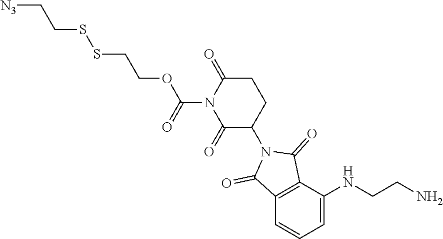 [SMILES: [N-]=[N+]=NCCSSCCOC(=O)N1C(=O)CCC(N2C(=O)c3cccc(NCCN)c3C2=O)C1=O]

To a solution of tert-butyl (2-((2-(2,6-dioxopiperidin-3-yl)-1,3-dioxoisoindolin-4-yl)amino)ethyl)carbamate (7) (see reference³ for the details of synthesis) (62.5 mg, 0.15 mmol, 1.0 equiv) in DMF (2 mL) was added NaH (9.0 mg, 60% in mineral oil, 0.225 mmol, 1.5 equiv) at 0° C. After stirring for 10 min, 2-((2-azidoethyl)disulfanyl)ethyl carbonochloridate (55.0 mg, 0.225 mmol, 1.5 equiv) was added to the mixture at 0° C. The reaction mixture was then warmed up to room temperature and stirred for additional 1 h. The resulting mixture was purified by preparative HPLC (10%-100% acetonitrile/0.1% TFA in H₂O). The product containing fractions were concentrated to remove the organic solvent under reduced pressure and then dried by lyophilization to afford desired intermediate as yellow solid. ESI m/z=644.2 [M+Na]⁺. To a solution of obtained above compound in CH₂Cl₂ (2 mL) was added TFA (1 mL) at room temperature. After stirring for 1 h, the resulting mixture was concentrated and purified by preparative HPLC (10%-100% acetonitrile/0.1% TFA in H₂O). The product containing fractions were concentrated to remove the organic solvent under reduced pressure and then dried by lyophilization to afford 8 as yellow solid in TFA salt form (26.0 mg, 27%). ¹H NMR (600 MHz, Methanol-d₄) δ 7.55-7.47 (m, 1H), 7.08-7.01 (m, 2H), 5.18-5.14 (m, 1H), 4.54-4.42 (m, 2H), 3.65-3.56 (m, 2H), 3.53-3.45 (m, 2H), 3.16-3.07 (m, 2H), 3.02-2.66 (m, 7H), 2.14-2.02 (m, 1H). ESI m/z=522.3 [M+H]⁺.

Synthesis of 2-((2-azidoethyl)disulfanyl)ethyl 3-(4-((2-(2-(4-(4-((5-chloro-4-((2-(isopropylsulfonyl)phenyl)amino)pyrimidin-2-yl)amino)-5-isopropoxy-2-methylphenyl)piperidin-1-yl)acetamido)ethyl)amino)-1,3-dioxoisoindolin-2-yl)-2,6-dioxopiperidine-1-carboxylate (9)

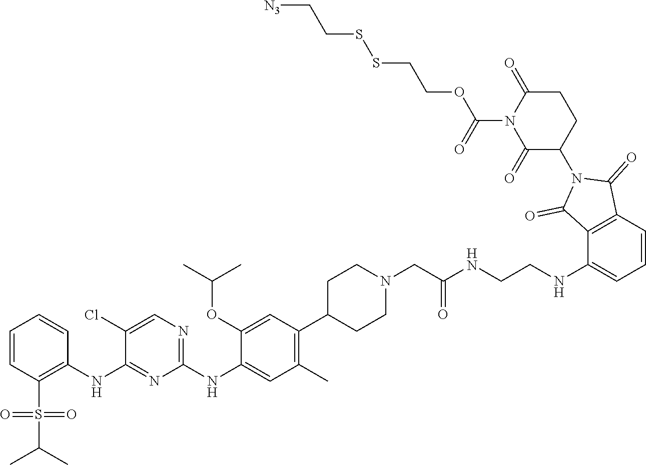 [SMILES: Cc1cc(Nc2ncc(Cl)c(Nc3ccccc3S(=O)(=O)C(C)C)n2)c(OC(C)C)cc1C1CCN(CC(=O)NCCNc2cccc3c2C(=O)N(C2CCC(=O)N(C(=O)OCCSSCCN=[N+]=[N-])C2=O)C3=O)CC1]

To a solution of compound 8 (26.0 mg, 0.041 mmol, 1.0 equiv) in DMSO (4 mL) were added compound 2-(4-(4-((5-chloro-4-((2-(isopropylsulfonyl)phenyl)amino)pyrimidin-2-yl)amino)-5-isopropoxy-2-methylphenyl)piperidin-1-yl)acetic acid (see reference³ for the details of synthesis) (27.8 mg, 0.045 mmol, 1.1 equiv), EDCI (1-ethyl-3-(3-dimethylaminopropyl)carbodiimide) (16.0 mg, 0.082 mmol, 2.0 equiv), HOAt (1-hydroxy-7-azabenzo-triazole) (11.2 mg, 0.082 mmol, 2.0 equiv) and NMM (N-Methylmorpholine) (20.7 mg, 0.205 mmol, 5.0 equiv). After being stirred at room temperature for 4 h, the resulting mixture was purified by preparative HPLC (10%-100% acetonitrile/0.1% TFA in H₂O). The product containing fractions were concentrated to remove the organic solvent under reduced pressure at room temperature and then dried by lyophilization to afford compound 9 as brown solid in TFA salt form (28.0 mg, 55%). ¹H NMR (600 MHz, Methanol-d₄) δ 8.26 (d, J=8.3 Hz, 1H), 8.14 (s, 1H), 7.89 (d, J=8.5 Hz, 1H), 7.63 (t, J=7.9 Hz, 1H), 7.53-7.47 (m, 2H), 7.39 (t, J=7.7 Hz, 1H), 7.08 (d, J=8.6 Hz, 1H), 7.01 (d, J=7.1 Hz, 1H), 6.79 (s, 1H), 5.12 (dd, J=12.8, 5.4 Hz, 1H), 4.54 (p, J=6.1 Hz, 1H), 4.40-4.29 (m, 2H), 3.84 (s, 2H), 3.63-3.42 (m, 8H), 3.34-3.25 (m, 1H), 3.11 (t, J=12.4 Hz, 2H), 3.04-2.96 (m, 1H), 2.93-2.82 (m, 3H), 2.82-2.72 (m, 3H), 2.65 (qd, J=13.3, 4.4 Hz, 1H), 2.17-1.84 (m, 8H), 1.24 (d, J=6.0 Hz, 6H), 1.17 (d, J=6.8 Hz, 6H). ESI m/z=1119.3 [M+H]⁺.

Synthesis of N²-(4-(((2-amino-4-oxo-3,4-dihydropteridin-6-yl)methyl)amino)benzoyl)-N⁵-((1-(2-((2-((3-(4-((2-(2-(4-(4-((5-chloro-4-((2-(isopropylsulfonyl)phenyl)amino)pyrimidin-2-yl)amino)-5-isopropoxy-2-methylphenyl)piperidin-1-yl)acetamido)ethyl)amino)-1,3-dioxoisoindolin-2-yl)-2,6-dioxopiperidine-1-carbonyl)oxy)ethyl)disulfanyl)ethyl)-1H-1,2,3-triazol-4-yl)methyl)-L-glutamine (FA-S2-MS4048)

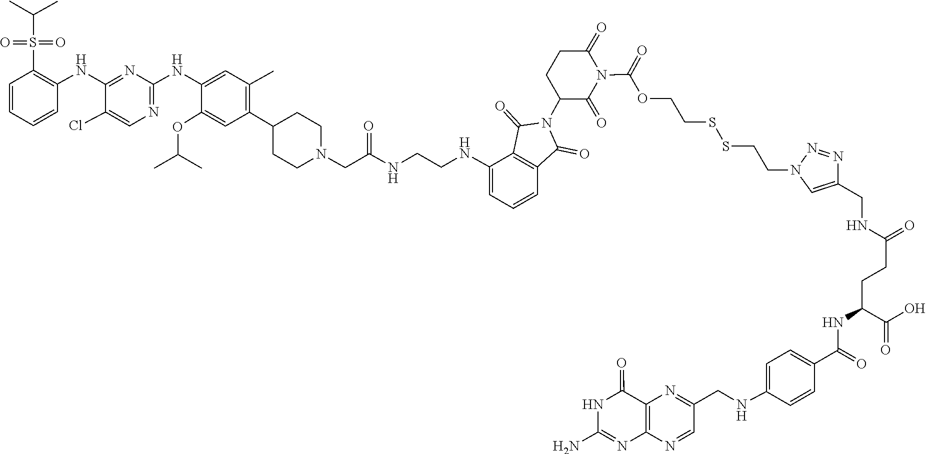 [SMILES: Cc1cc(Nc2ncc(Cl)c(Nc3ccccc3S(=O)(=O)C(C)C)n2)c(OC(C)C)cc1C1CCN(CC(=O)NCCNc2cccc3c2C(=O)N(C2CCC(=O)N(C(=O)OCCSSCCn4cc(CNC(=O)CC[C@H](NC(=O)c5ccc(NCc6cnc7nc(N)[nH]c(=O)c7n6)cc5)C(=O)O)nn4)C2=O)C3=O)CC1]

To a solution of compound 9 (28.0 mg, 0.023 mmol, 1.0 equiv) in DMF (1.6 mL)/water (0.8 mL) were added compound 4 (13.0 mg, 0.027 mmol, 1.2 equiv), sodium ascorbate (1.8 mg, 0.009 mmol, 0.4 equiv) and CuSO₄·5H₂O (1.2 mg, 0.005 mmol, 0.2 equiv) at room temperature. The reaction mixture was heated to 50° C. After being stirred at 50° C. for 2 h, the resulting mixture was purified by preparative HPLC (10%-100% acetonitrile/0.1% TFA in H₂O). The product containing fractions were concentrated to remove the organic solvent under reduced pressure at room temperature and then dried by lyophilization to afford FA-S2-MS4048 as yellow solid in TFA salt form (12.7 mg, 32%). ¹H NMR (600 MHz, DMSO-d₆) δ 9.67 (s, 1H), 9.55 (s, 1H), 8.85 (t, J=5.8 Hz, 1H), 8.70 (s, 1H), 8.43 (d, J=8.2 Hz, 1H), 8.37 (t, J=5.8 Hz, 1H), 8.28 (s, 1H), 8.24 (s, 1H), 8.21 (d, J=7.7 Hz, 1H), 7.92 (s, 1H), 7.86 (d, J=8.0 Hz, 1H), 7.69-7.58 (m, 4H), 7.52 (s, 1H), 7.38 (t, J=7.6 Hz, 1H), 7.22 (d, J=8.7 Hz, 1H), 7.07 (d, J=7.1 Hz, 1H), 6.80 (s, 2H), 6.65 (d, J=8.5 Hz, 2H), 5.39 (dd, J=12.9, 5.6 Hz, 1H), 4.63-4.46 (m, 7H), 4.36-4.20 (m, 3H), 3.94 (s, 2H), 3.58-3.34 (m, 8H), 3.28-3.16 (m, 3H), 3.16-3.07 (m, 1H), 3.04 (t, J=6.3 Hz, 2H), 2.99-2.90 (m, 1H), 2.85-2.77 (m, 1H), 2.66-2.55 (m, 1H), 2.30-2.18 (m, 2H), 2.18-2.00 (m, 7H), 1.94-1.83 (m, 3H), 1.26 (d, J=6.0 Hz, 6H), 1.16 (d, J=6.8 Hz, 6H). ¹³C NMR (151 MHz, DMSO-d₆) δ 174.30, 171.99, 170.55, 168.91, 168.28, 167.48, 166.82, 165.08, 160.65, 157.92, 155.63, 154.95, 153.76, 151.16, 150.92, 150.53, 148.68, 147.28, 146.79, 145.66, 138.34, 138.11, 136.91, 135.27, 132.60, 131.48, 129.47, 128.45, 127.45, 127.19, 125.42, 124.54, 124.47, 124.36, 123.38, 121.87, 117.71, 111.83, 111.69, 111.38, 109.67, 104.91, 71.34, 67.19, 57.38, 55.27, 53.56, 52.54, 48.80, 48.38, 46.30, 41.73, 40.51, 38.64, 37.42, 35.87, 34.67, 32.25, 31.18, 29.52, 26.93, 22.34, 21.76, 18.79, 15.30. HRMS (ESI-TOF) calcd for C₇₂H₈₂ClN₂₆O₁₅S₃⁺ [M+H]⁺ 1597.5114, found 1597.5101. HPLC>98%, t_{R}=4.35 min.

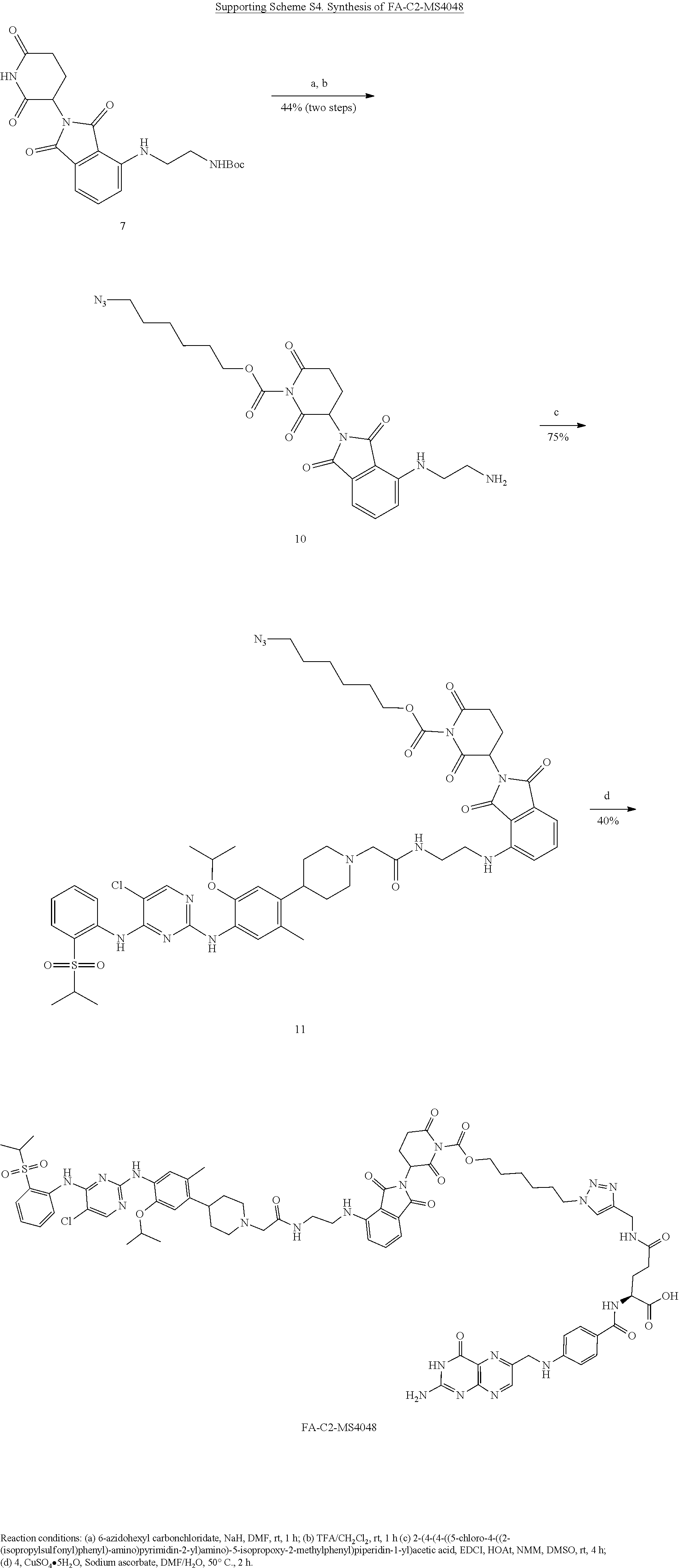 [SMILES: CC(C)(C)OC(=O)NCCNc1cccc2c1C(=O)N(C1CCC(=O)NC1=O)C2=O ; [N-]=[N+]=NCCCCCCOC(=O)N1C(=O)CCC(N2C(=O)c3cccc(NCCN)c3C2=O)C1=O ; Cc1cc(Nc2ncc(Cl)c(Nc3ccccc3S(=O)(=O)C(C)C)n2)c(OC(C)C)cc1C1CCN(CC(=O)NCCNc2cccc3c2C(=O)N(C2CCC(=O)N(C(=O)OCCCCCCN=[N+]=[N-])C2=O)C3=O)CC1 ; Cc1cc(Nc2ncc(Cl)c(Nc3ccccc3S(=O)(=O)C(C)C)n2)c(OC(C)C)cc1C1CCN(CC(=O)NCCNc2cccc3c2C(=O)N(C2CCC(=O)N(C(=O)OCCCCCCn4cc(CNC(=O)CC[C@H](NC(=O)c5ccc(NCc6cnc7nc(N)[nH]c(=O)c7n6)cc5)C(=O)O)nn4)C2=O)C3=O)CC1]

Synthesis of 6-azidohexyl 3-(4-((2-aminoethyl)amino)-1,3-dioxoisoindolin-2-yl)-2,6-dioxopiperidine-1-carboxylate (10)

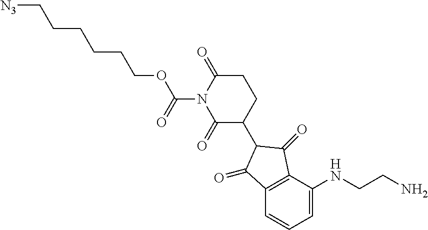 [SMILES: [N-]=[N+]=NCCCCCCOC(=O)N1C(=O)CCC(C2C(=O)c3cccc(NCCN)c3C2=O)C1=O]

To a solution of tert-butyl (2-((2-(2,6-dioxopiperidin-3-yl)-1,3-dioxoisoindolin-4-yl)amino)ethyl)carbamate (7) (62.5 mg, 0.15 mmol, 1.0 equiv) in DMF (2 mL) was added NaH (9.0 mg, 60% in mineral oil, 0.225 mmol, 1.5 equiv) at 0° C. After stirring for 10 min, 2-((2-azidoethyl)disulfanyl)ethyl carbonochloridate (46.3 mg, 0.225 mmol, 1.5 equiv) was added to the mixture at 0° C. The reaction mixture was then warmed up to room temperature and stirred for additional 1 h. The resulting mixture was purified by preparative HPLC (10%-100% acetonitrile/0.1% TFA in H₂O). The product containing fractions were concentrated to remove the organic solvent under reduced pressure and then dried by lyophilization to afford desired intermediate as yellow solid. ESI m/z=608.3 [M+Na]⁺. To a solution of obtained above compound in CH₂Cl₂ (2 mL) was added TFA (1 mL) at room temperature. After stirring for 1 h, the resulting mixture was concentrated and purified by preparative HPLC (10%-100% acetonitrile/0.1% TFA in H₂O). The product containing fractions were concentrated to remove the organic solvent under reduced pressure and then dried by lyophilization to afford 10 as yellow solid in TFA salt form (40.0 mg, 44%). ¹H NMR (600 MHz, Methanol-d₄) δ 7.62 (dd, J=8.5, 7.2 Hz, 1H), 7.15 (d, J=7.8 Hz, 2H), 5.24 (dd, J=12.9, 5.4 Hz, 1H), 4.38-4.30 (m, 2H), 3.69 (t, J=6.1 Hz, 2H), 3.26 (t, J=6.9 Hz, 2H), 3.20 (t, J=6.1 Hz, 2H), 3.01 (ddd, J=17.6, 13.7, 5.3 Hz, 1H), 2.91 (ddd, J=17.6, 4.5, 2.7 Hz, 1H), 2.79 (qd, J=13.2, 4.5 Hz, 1H), 2.19-2.11 (m, 1H), 1.76-1.66 (m, 2H), 1.57 (p, J=7.0 Hz, 2H), 1.47-1.33 (m, 4H). ESI m/z=486.3 [M+H]⁺.

Synthesis of 6-azidohexyl 3-(4-((2-(2-(4-(4-((5-chloro-4-((2-(isopropylsulfonyl)phenyl)amino)pyrimidin-2-yl)amino)-5-isopropoxy-2-methylphenyl)piperidin-1-yl)acetamido)ethyl)amino)-1,3-dioxoisoindolin-2-yl)-2,6-dioxopiperidine-1-carboxylate (11)

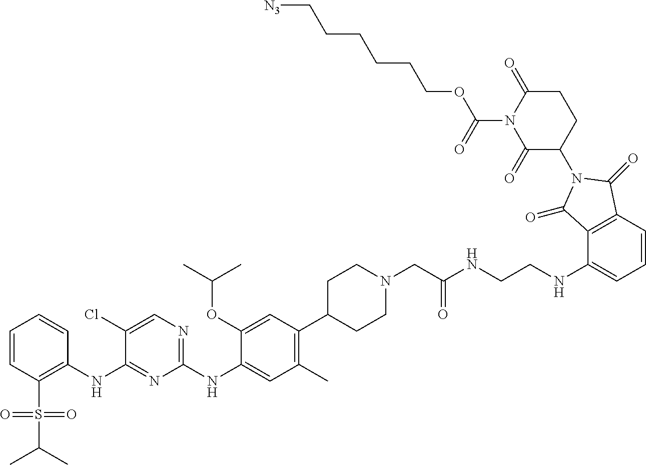 [SMILES: Cc1cc(Nc2ncc(Cl)c(Nc3ccccc3S(=O)(=O)C(C)C)n2)c(OC(C)C)cc1C1CCN(CC(=O)NCCNc2cccc3c2C(=O)N(C2CCC(=O)N(C(=O)OCCCCCCN=[N+]=[N-])C2=O)C3=O)CC1]

To a solution of compound 10 (40.0 mg, 0.067 mmol, 1.0 equiv) in DMSO (4 mL) were added compound 2-(4-(4-((5-chloro-4-((2-(isopropylsulfonyl)phenyl)amino)pyrimidin-2-yl)amino)-5-isopropoxy-2-methylphenyl)piperidin-1-yl)acetic acid (53.3 mg, 0.073 mmol, 1.1 equiv), EDCI (25.7 mg, 0.134 mmol, 2.0 equiv), HOAt (18.2 mg, 0.134 mmol, 2.0 equiv) and NMM (34.0 mg, 0.335 mmol, 5.0 equiv). After being stirred at room temperature for 4 h, the resulting mixture was purified by preparative HPLC (10%-100% acetonitrile/0.1% TFA in H₂O). The product containing fractions were concentrated to remove the organic solvent under reduced pressure and then dried by lyophilization to afford compound 11 as brown solid in TFA salt form (60.1 mg, 75%). ¹H NMR (600 MHz, Methanol-d₄) δ 8.21 (d, J=8.3 Hz, 1H), 8.12 (s, 1H), 7.87 (dd, J=7.9, 1.6 Hz, 1H), 7.64-7.56 (m, 1H), 7.47 (dd, J=8.6, 7.1 Hz, 1H), 7.43-7.34 (m, 2H), 7.05 (d, J=8.6 Hz, 1H), 6.97 (d, J=7.1 Hz, 1H), 6.78 (s, 1H), 5.09 (dd, J=12.8, 5.4 Hz, 1H), 4.53 (p, J=6.1 Hz, 1H), 4.18-4.04 (m, 2H), 3.83 (s, 2H), 3.56 (t, J=11.0 Hz, 2H), 3.47 (t, J=5.9 Hz, 2H), 3.44-3.40 (m, 2H), 3.33-3.22 (m, 1H), 3.16-3.08 (m, 4H), 3.02-2.96 (m, 1H), 2.86 (ddd, J=17.5, 13.7, 5.3 Hz, 1H), 2.74 (ddd, J=17.6, 4.5, 2.8 Hz, 1H), 2.62 (qd, J=13.2, 4.4 Hz, 1H), 2.07 (s, 3H), 2.02-1.84 (m, 5H), 1.57-1.49 (m, 2H), 1.42 (p, J=7.0 Hz, 2H), 1.29-1.22 (m, 4H), 1.21 (d, J=6.0 Hz, 6H), 1.14 (d, J=6.8 Hz, 6H). ESI m/z=1083.5 [M+H]⁺.

Synthesis of N²-(4-(((2-amino-4-oxo-3,4-dihydropteridin-6-yl)methyl)amino)benzoyl)-N⁵-((1-(6-((3-(4-((2-(2-(4-(4-((5-chloro-4-((2-(isopropylsulfonyl)phenyl)amino)pyrimidin-2-yl)amino)-5-isopropoxy-2-methylphenyl)piperidin-1-yl)acetamido)ethyl)amino)-1,3-dioxoisoindolin-2-yl)-2,6-dioxopiperidine-1-carbonyl)oxy)hexyl)-1H-1,2,3-triazol-4-yl)methyl)-L-glutamine (FA-C2-MS4048)

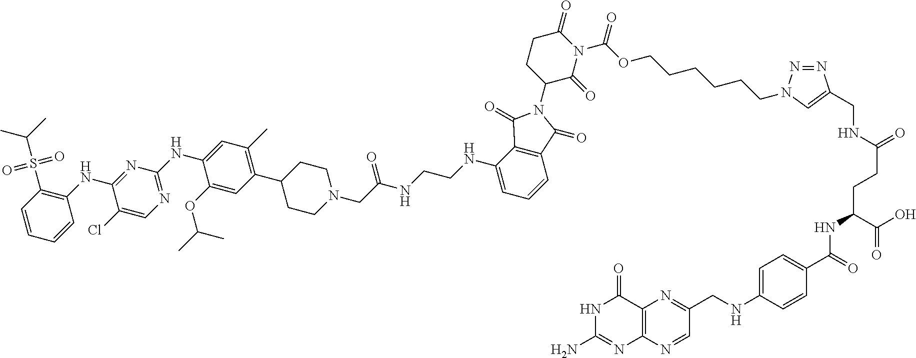 [SMILES: Cc1cc(Nc2ncc(Cl)c(Nc3ccccc3S(=O)(=O)C(C)C)n2)c(OC(C)C)cc1C1CCN(CC(=O)NCCNc2cccc3c2C(=O)N(C2CCC(=O)N(C(=O)OCCCCCCn4cc(CNC(=O)CC[C@H](NC(=O)c5ccc(NCc6cnc7nc(N)[nH]c(=O)c7n6)cc5)C(=O)O)nn4)C2=O)C3=O)CC1]

To a solution of compound 11 (26.0 mg, 0.022 mmol, 1.0 equiv) in DMF (1.6 mL)/water (0.8 mL) were added compound 4 (12.5 mg, 0.026 mmol, 1.2 equiv), sodium ascorbate (1.7 mg, 0.009 mmol, 0.4 equiv) and CuSO₄·5H₂O (1.1 mg, 0.004 mmol, 0.2 equiv) at room temperature. The reaction mixture was heated to 50° C. After being stirred at 50° C. for 2 h, the resulting mixture was purified by preparative HPLC (10%-100% acetonitrile/0.1% TFA in H₂O). The product containing fractions were concentrated to remove the organic solvent under reduced pressure at room temperature and then dried by lyophilization to afford FA-C2-MS4048 as yellow solid in TFA salt form (14.9 mg, 40%). ¹H NMR (600 MHz, DMSO-d₆) δ 9.60 (s, 1H), 9.50 (s, 1H), 8.77 (t, J=5.7 Hz, 1H), 8.64 (s, 1H), 8.35 (d, J=8.3 Hz, 1H), 8.26 (t, J=5.8 Hz, 1H), 8.24-8.19 (m, 2H), 8.14 (d, J=7.6 Hz, 1H), 7.85-7.74 (m, 2H), 7.64-7.51 (m, 4H), 7.44 (s, 1H), 7.31 (t, J=7.6 Hz, 1H), 7.14 (d, J=8.6 Hz, 1H), 6.99 (d, J=7.1 Hz, 1H), 6.73 (s, 2H), 6.58 (d, J=8.3 Hz, 2H), 5.29 (dd, J=12.8, 5.6 Hz, 1H), 4.54-4.39 (m, 3H), 4.30-4.11 (m, 7H), 3.86 (s, 2H), 3.53-3.26 (m, 7H), 3.20-3.08 (m, 2H), 3.07-2.98 (m, 1H), 2.88 (t, J=12.3 Hz, 1H), 2.75-2.66 (m, 1H), 2.57-2.46 (m, 1H), 2.27-2.09 (m, 2H), 2.11-1.94 (m, 7H), 1.91-1.81 (m, 1H), 1.81-1.74 (m, 2H), 1.72-1.65 (m, 2H), 1.62-1.47 (m, 2H), 1.34-1.12 (m, 10H), 1.09 (d, J=6.8 Hz, 6H). ¹³C NMR (151 MHz, DMSO-d₆) δ 174.31, 171.97, 170.58, 168.91, 168.31, 167.47, 166.79, 165.08, 160.40, 157.71, 155.74, 154.54, 153.58, 151.13, 150.90, 148.56, 147.28, 146.78, 145.40, 138.27, 138.19, 136.88, 135.27, 132.60, 131.49, 129.45, 128.42, 127.33, 127.18, 125.58, 124.60, 124.54, 124.47, 123.02, 121.91, 117.70, 111.81, 111.70, 111.35, 109.67, 104.92, 71.34, 69.60, 57.37, 55.26, 53.55, 52.55, 49.56, 48.78, 46.27, 41.75, 40.50, 38.63, 34.73, 34.66, 32.25, 31.16, 30.00, 29.51, 27.93, 26.93, 25.76, 24.75, 22.33, 21.81, 18.78, 15.29. HRMS (ESI-TOF) calcd for C₇₄H₈₆ClN₂₀O₁₅S⁺[M+H]⁺1561.5985, found 1561.5961. HPLC>97%, t_{R}=4.31 min.

As various changes can be made in the above-described subject matter without departing from the scope and spirit of the present disclsoure, it is intended that all subject matter contained in the above description, or defined in the appended claims, be interpreted as descriptive and illustrative. Many modifications and variations of the present disclosure are possible in light of the above teachings. Accordingly, the present description is intended to embrace all such alternatives, modifications and variances which fall within the scope of the appended claims.

What is claimed is:

1. A targeting group conjugated PROTAC, wherein ubiquitin recruitment for the PROTAC only occurs following hydrolytic or reductive cleavage of the targeting group.

2. The targeting group conjugated PROTAC of claim 1, wherein said PROTAC is conjugated to folate, fluorodeoxyglucose or biotin moiety.

3. A compound having the structure of formula (I):
PB-L₁-ULB-L₂-TG (I)
wherein ULB is a ubiquitin ligase binding moiety;
L₁ is absent or a linker;
L₂ is absent or a linker;
PB is a protein binding moiety; and
TG is a targeting group that preferentially binds to a protein with increased expression in a neoplastic cell as compared to an otherwise identical healthy cell;
wherein ubiquitin ligase binding potential of said compound is increased following cleavage between the ULB group and the TG group;
or pharmaceutically acceptable salts thereof.

4. The compound of claim 3, wherein TG is a folate derivative or a fluorodeoxyglucose derivative, or a biotin derivative.

5. The compound of claim 4, wherein said TG group is a folate derivative having the structure of formula (II): 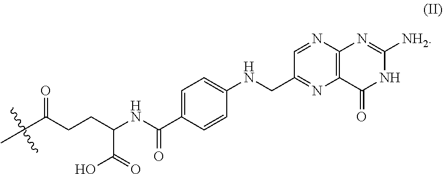 [SMILES: *C(=O)CCC(NC(=O)c1ccc(NCc2cnc3nc(N)[nH]c(=O)c3n2)cc1)C(=O)O]
wherein 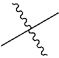
is the point of attachment to the compound.

6. The compound of claim 4, wherein said TG group is a folate derivative having the structure of formula (IIa) or (IIb): 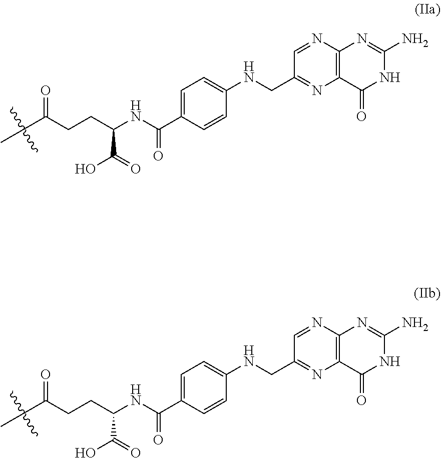 [SMILES: *C(=O)CC[C@@H](NC(=O)c1ccc(NCc2cnc3nc(N)[nH]c(=O)c3n2)cc1)C(=O)O ; *C(=O)CC[C@H](NC(=O)c1ccc(NCc2cnc3nc(N)[nH]c(=O)c3n2)cc1)C(=O)O]
wherein 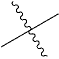
is the point of attachment to the compound.

7. The compound of claim 3, wherein -L₂-TG is conjugated to ULB through a hydroxyl group required for ubiquitin ligase binding.

8. The compound of claim 7, wherein the conjugation through a hydroxyl group is ester conjugation.

9. The compound of claim 3, wherein said ULB binds to an E3 ubiquitin ligase following cleavage.

10. The compound of claim 9, wherein the E3 ubiquitin ligase is selected from the group consisting of von Hippel Lindau (VHL) E3 ubiquitin ligase, β-Transducin Repeat Containing (β-TRCP) E3 Ubiquitin Protein Ligase, Mouse Double Minute 2 (Mdm2) E3 Ubiquitin Protein Ligase, and a Cereblon (CRBN) E3 Ubiquitin ligase.

11. The compound of claim 1, wherein said compound has the structure of formula (III):

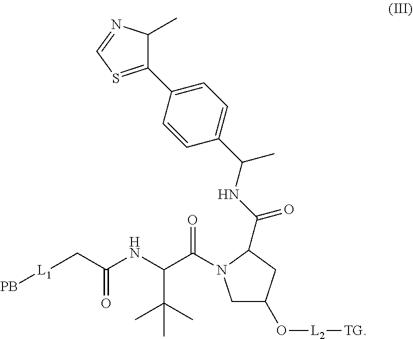

12. The compound of claim 11, wherein said compound has the structure of formula (IIIa), (IIIb), (IIIc), (IIId), or (IIIe):

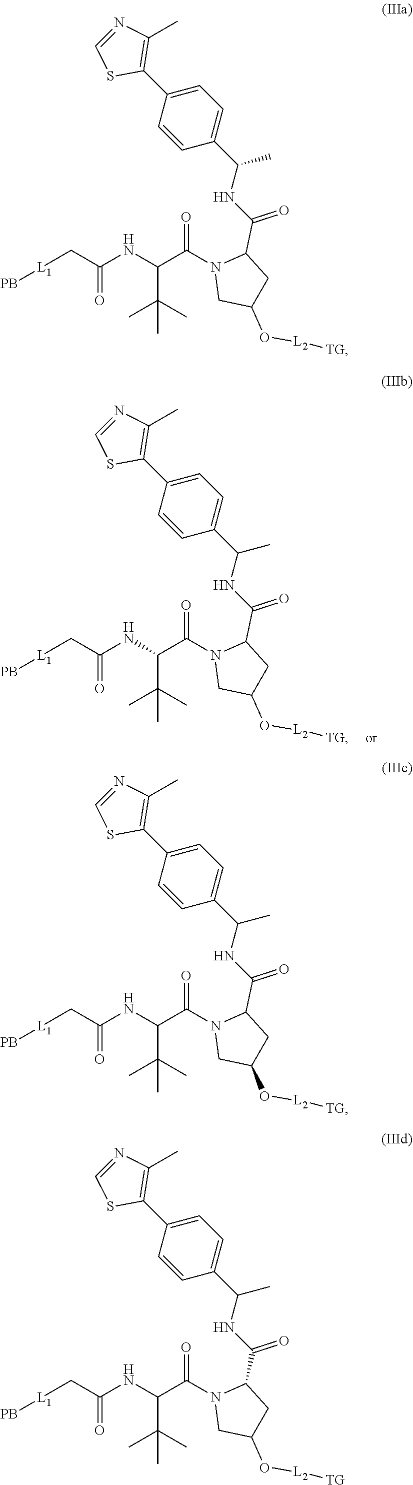

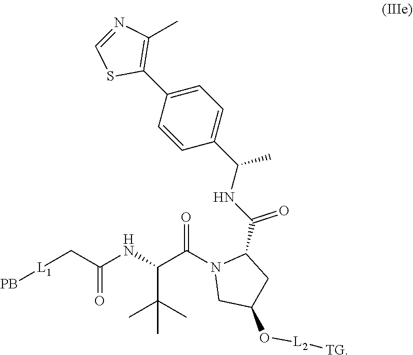

13. The compound of claim 1, wherein L₂ comprises a heteroarylene group, —C(O)—, —NH—, or combinations thereof.

14. The compound of claim 1, wherein the compound has the structure of formula (IV): 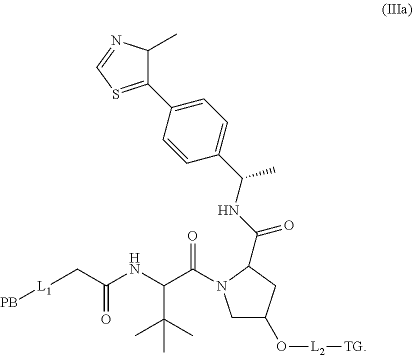
wherein X is absent (i.e, it is a bond) or may comprise the remaining portions of the ULB moiety (e.g., optionally substituted isoindolin-1-one such as 4-aminoisoindolin-1-one, optionally substituted isoindolin,1-3-dione such as 4-aminoisoindolin1,3,dione) which is conjugated to the L₁ moiety.

15. The compound of claim 1, wherein said compound has the structure of formula (V):
PB-L₁-ULB—X₁—X₂—X₃—X₄—X₅—X₆—X₇-TG (V)
wherein X₁-X₇ are independently selected from
absent, —C(O)—, —O—, —OC(O)—, —N(R^{a})C(O)—, —(C(R^{a})(R^{a}))₁₋₈—, —(C(R^{a})(R^{a})C(R^{a})(R^{a})O)₁₋₈—, —S—S—, arylene, and heteroarylene; and
R^{a} is independently selected at each occurrence from hydrogen and alkyl.

16. The compound of claim 15, wherein one of X₃-X₅ is

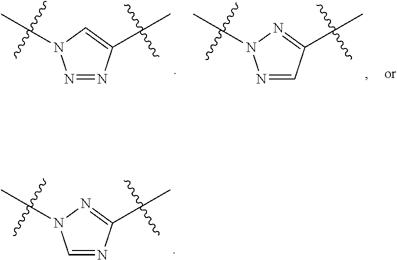 [SMILES: *c1cn(*)nn1 ; *c1cnn(*)n1 ; *c1ncn(*)n1]

17. The compound of claim 1, wherein said compound has the structure of one of the following:
formula (Vb): 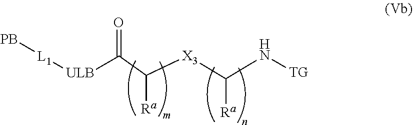
wherein m and n are independently selected from 1-8 and
X₃ is heteroarylene formula (Vc): 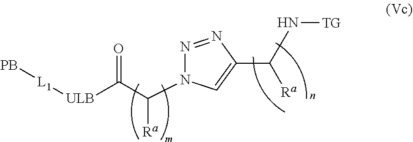
formula (Vd): 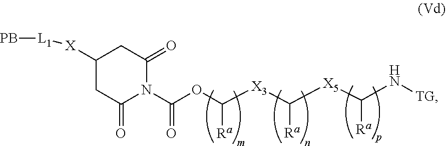
wherein m, n, and p are independently an intenger selected from 0-8 (i.e., 0, 1, 2, 3, 4, 5, 6, 7, and 8); and
X₃ or X₅ is —S—S—.
formula (VI), (VIa), or (VIb): 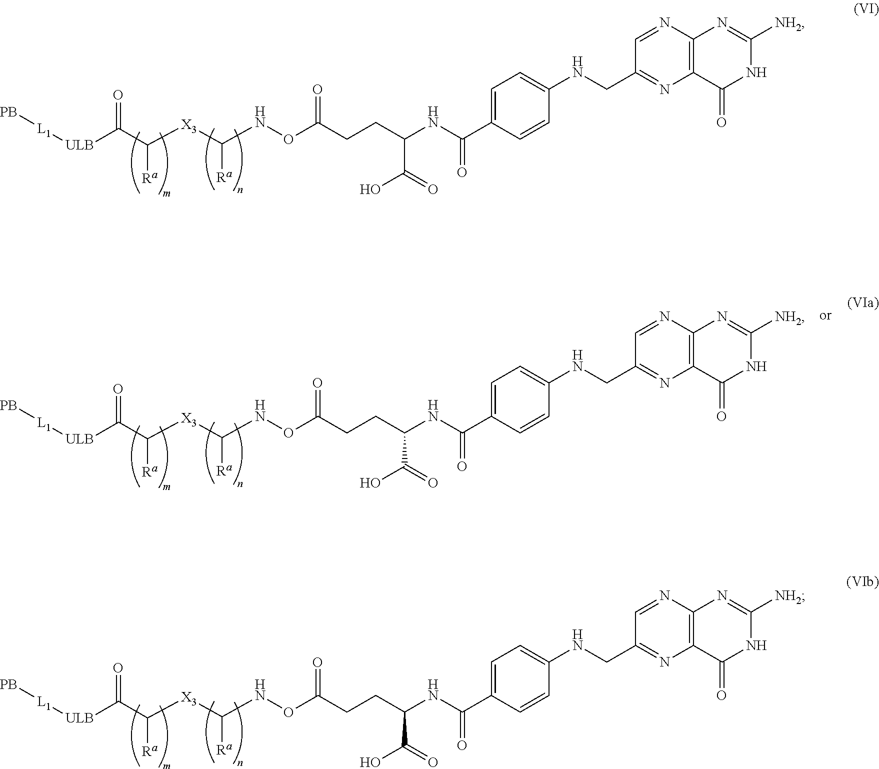
wherein m and n are independently an integer selected from 0-8;
X₃ is heteroarylene; and
R^{a} is independently selected at each occurrence from hydrogen and alkyl.

18. The compound of claim 3, wherein PB is: 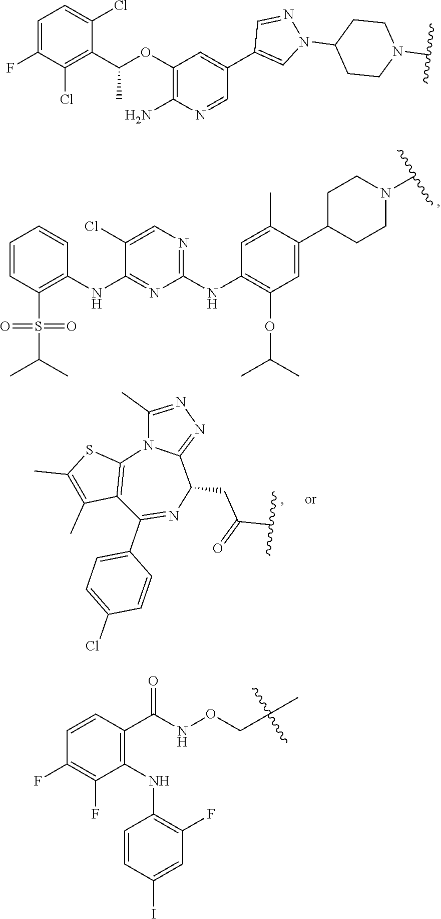 [SMILES: *N1CCC(n2cc(-c3cnc(N)c(O[C@H](C)c4c(Cl)ccc(F)c4Cl)c3)cn2)CC1 ; *N1CCC(c2cc(OC(C)C)c(Nc3ncc(Cl)c(Nc4ccccc4S(=O)(=O)C(C)C)n3)cc2C)CC1 ; *C(=O)C[C@@H]1N=C(c2ccc(Cl)cc2)c2c(sc(C)c2C)-n2c(C)nnc21 ; *CONC(=O)c1ccc(F)c(F)c1Nc1ccc(I)cc1F]
wherein 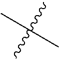
indicates the point of attachment to the L₁ group.

19. The compound of claim 3, wherein L₁ has the structure of formula (VI):
—Y₁—Y₂—Y₃—Y₄— (VI)
wherein Y₁-Y₄ are independently selected from
absent, —C(O)—, —O—, —OC(O)—, —NR^{a}—, —N(R^{a})C(O)—, —(C(R^{a})(R^{a}))₁₋₁₂, —(C(R^{a})(R^{a})C(R^{a})(R^{a})O)₁₋₁₂—, and —S—S—; and
R^{a} is independently selected at each occurrence from hydrogen and alkyl.

20. The compound of claim 21, wherein Y₄ is —(C(R^{a})(R^{a}))₁₋₁₂— or —(C(R^{a})(R^{a})C(R^{a})(R^{a})O)₁₋₁₂—.

21. The compound of claim 3, wherein said compound has the structure of formula (VIIa), (VIIb), (VIIc), (VIId), (VIIf), (VIIg), (VIIh), (VIIi), (VIIj), (VIIk), (VIIl), (VIIm), or (VIIn):
PB—NH—(CH₂)₁₋₁₀—NH—C(O)—ULB-L₂-TG (VIIa)
PB—(CH₂)₁₋₁₀—NH—C(O)—(CH₂)₁₋₁₀—ULB-L₂-TG (VIIb)
PB—(CH₂)₁₋₁₀—NH—(CH₂)₁₋₁₀—ULB-L₂-TG (VIIc)
PB—NH—(CH₂)₁₋₁₀—NH—C(O)—ULB-L₂-TG (VIId)
PB—C(O)—(CH₂)₁₋₁₀—ULB-L₂-TG (VIIg)
PB—NH—(CH₂)₁₋₁₀—ULB-L₂-TG (VIIg)
PB—NH—(CH₂CH₂O)₁₋₁₀—NH—C(O)—ULB-L₂-TG (VIIh)
PB—(CH₂CH₂O)₁₋₁₀—NH—C(O)—(CH₂CH₂O)₁₋₁₀—ULB-L₂-TG (VIIi)
PB—(CH₂CH₂O)₁₋₁₀—NH—(CH₂CH₂O)₁₋₁₀—ULB-L₂-TG (VIIj)
PB—NH—(CH₂CH₂O)₁₋₁₀—NH—C(O)—ULB-L₂-TG (VIIk)
PB—C(O)—(CH₂CH₂O)₁₋₁₀—ULB-L₂-TG (VIIl)
PB—NH—(CH₂CH₂O)₁₋₁₀—ULB-L₂-TG (VIIm)
PB—(CH₂)₁₋₁₀—C(O)—NH—(CH₂)₁₋₁₀—ULB-L₂-TG (VIIn)

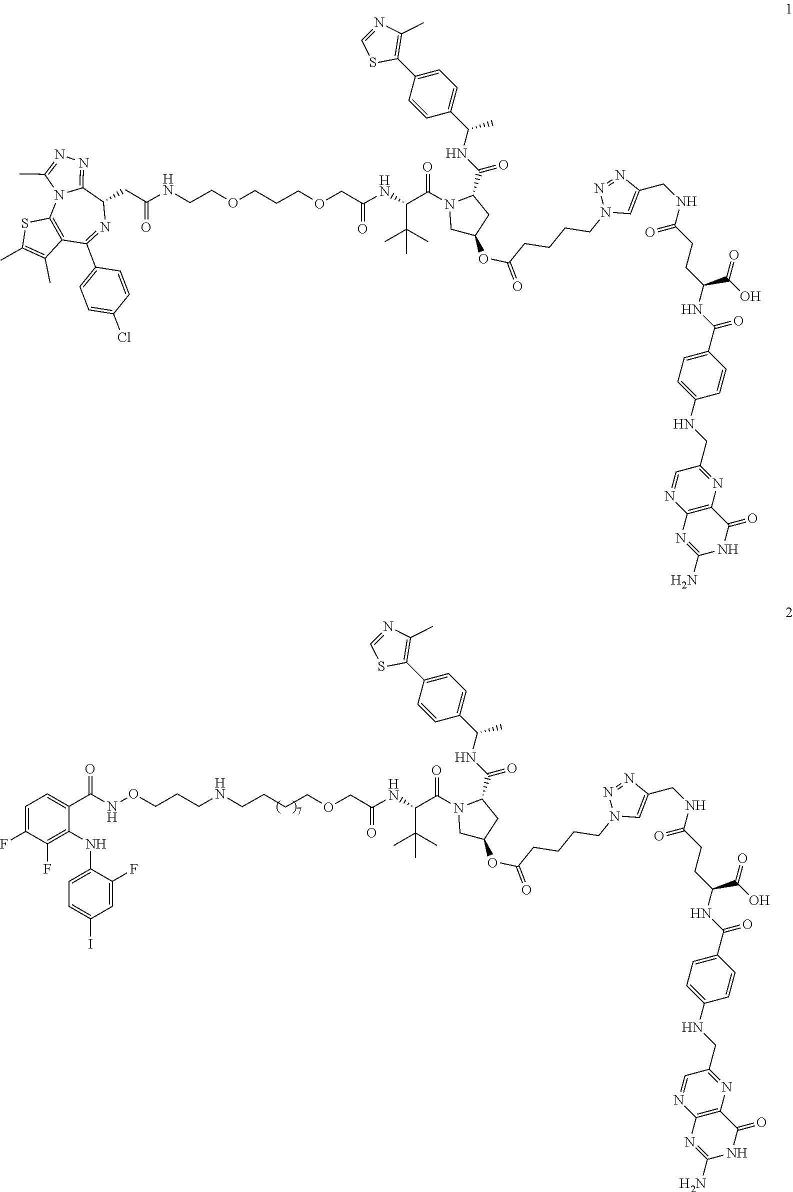

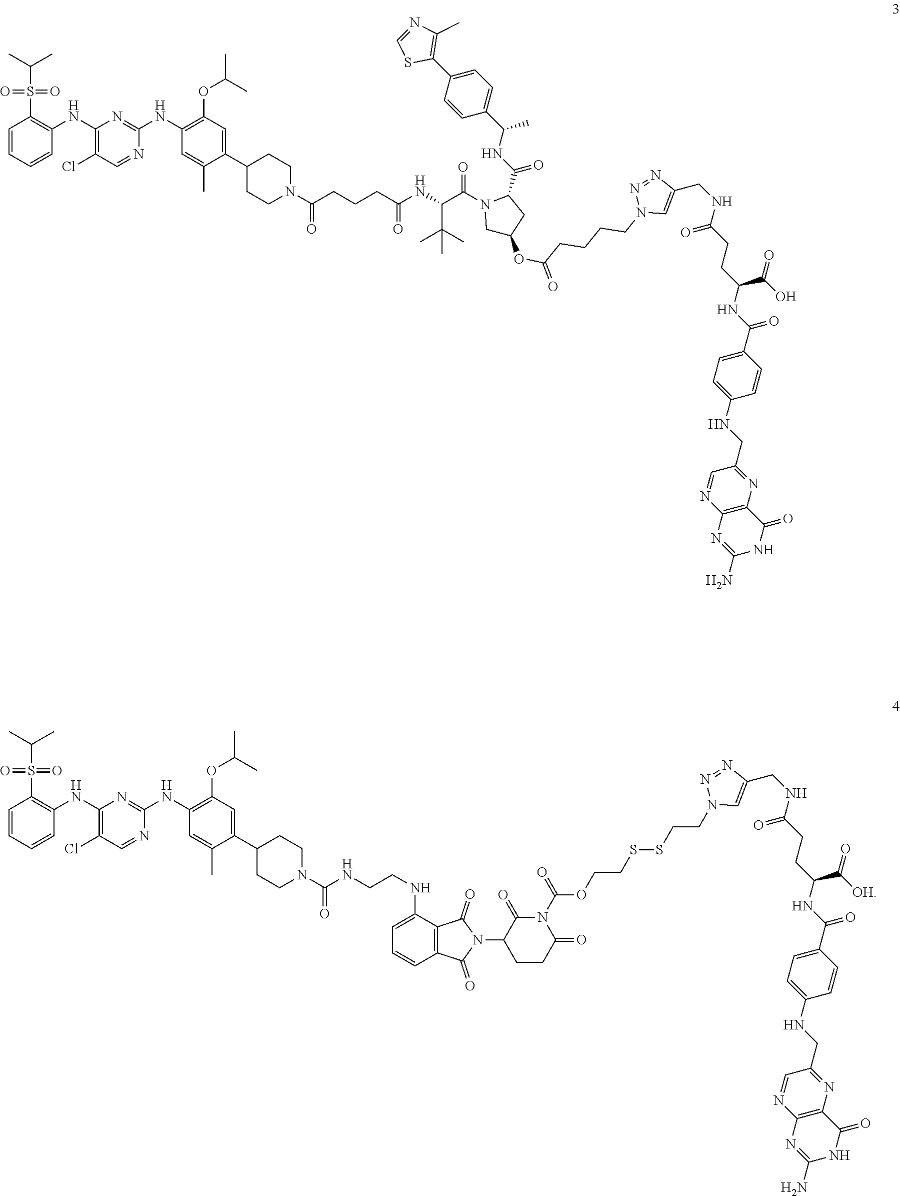

22. A method for degrading a protein of interest, the method comprising contacting the protein of interest with a compound of claim 1 and activating the compound through hydrolysis to cleave the targeting group from the ubiquitin ligase binding group and increase binding affinity of the compound for ubiquitin ligase.


SEQUENCE LISTING (3 sequences)

SEQ ID NO: 1 - DNA, 20 bp, synthetic construct
  misc_feature 1..20: note = Description of Artificial Sequence: Synthetic oligonucleotide
  source 1..20: mol_type = other DNA
taaacagacatggccggcga

SEQ ID NO: 2 - DNA, 20 bp, synthetic construct
  misc_feature 1..20: note = Description of Artificial Sequence: Synthetic oligonucleotide
  source 1..20: mol_type = other DNA
gtcctgctgatctccttcgc

SEQ ID NO: 3 - DNA, 20 bp, synthetic construct
  misc_feature 1..20: note = Description of Artificial Sequence: Synthetic oligonucleotide
  source 1..20: mol_type = other DNA
caggacgctgcgcacaacat